(19) United States
(12) Patent Application Publication
(10) Pub. No.: US 2007/0028314 A1
(43) Pub. Date: Feb. 1, 2007

(54) Bone and/or joint disease-associated genes

(76) Inventors: Toshihisa Komori, Nagasaki (JP); Naoko Kanatani, Nagasaki (JP); Carolina Andrea Yoshida, Nagasaki (JP); Akira Zanma, Tokyo (JP); Shinji Kobayashi, Tokyo (JP); Kei Yamana, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/576,496

(22) PCT Filed: Oct. 20, 2004
(86) PCT No.: PCT/JP04/15879

§ 371 Date: Apr. 20, 2006

(30) Foreign Application Priority Data
Oct. 20, 2003 (JP) 2003-359172

(51) Int. Cl. A01K 67/027; C12Q 1/68; C07H 21/04; C12N 5/06; C07K 14/705

(52) U.S. Cl. 800/18; 514/44; 435/354; 435/6; 536/23.2; 530/350

(57) ABSTRACT

This invention provides a method for obtaining a gene involved in regulation of cartilage differentiation, in which a transcription factor, preferably Runx2/Cbfa1, is forcedly expressed in a cell that is deficient such transcription factor, preferably in a Runx2/Cbfa1-deficient chondrocyte, and the gene, the expression of which is thereby induced, is selected using DNA chip analysis, subtraction, or other means as well as a Runx2/Cbfa1-deficient chondrocyte useful for carrying out such method. The invention also provides a polynucleotide obtained by such method, a polypeptide encoded by such polynucleotide, an antibody against such polypeptide, a recombinant vector comprising such polynucleotide, a transformant comprising such recombinant DNA vector, a cell expressing such polypeptide, a transgenic animal of such polynucleotide, an animal model of a bone and/or joint disease (preferably osteoarthritis), a method for screening for a therapeutic agent and/or prophylactic agent for a bone and/or joint disease (preferably osteoarthritis) using the aforementioned objects, a candidate compound for a therapeutic agent and/or prophylactic agent selected by such method, a pharmaceutical composition for a bone and/or joint disease (preferably osteoarthritis), and a method for diagnosing such disease.

### TECHNICAL FIELD

The present invention relates to proteins having a novel function of regulating cartilage differentiation, genes encoding such proteins, and a method for isolating such genes. Further, the present invention relates to a method for screening for therapeutic agents for a bone and/or joint disease (preferably osteoarthritis) as well as therapeutic agents for such diseases.

### BACKGROUND ART

Osteoarthritis (OA), which is a bone and/or joint disease, causes cartilage degeneration and spur formation in the course of aging or receipt of dynamic stress, and OA involves a secondary synovitis. Besides aging, sexuality (female), obesity, and external injuries (such as ligament or medial meniscus injuries) can be risk factors, although causal factors of OA have not been clarified. It is reported that approximately 900,000 people are newly afflicted with osteoarthritis per year in Japan (Non-Patent Document No. 1: Nankodo, Co., Ltd., Orthopedic surgery 42: 2-6, 2002). It is considered that the number of patients will grow as the aging of the population progresses in the future. For the conservative medical management for osteoarthritis, a nonsteroidal anti-inflammatory drug is generally administered orally to a patient, and a high-molecular-weight hyaluronic acid is injected intraarticularly into a patient. However, no medicine having a mechanism that clearly inhibits cartilage degeneration and stimulates cartilage regeneration has yet been developed. Since causal factors of osteoarthritis have not yet been fully elucidated, development of such medicine necessitates the identification of genes that are deeply involved in the advancement of osteoarthritis.

In the past, human disease-associated genes the expression levels of which differ between the human pathologic tissues and the human normal tissues at the site of a primary lesion were identified via subtraction, DNA microarray analysis, differential display, EST comparison, or other means for obtaining such genes. In the case of one of bone and/or joint diseases (i.e., osteoarthritis), for example, 5,000 expressed sequenced tags (ESTs) from human normal and osteoarthritic cartilage cDNA libraries were compared, and the expression frequencies thereof were compared to identify the EST, the expression levels of which are increased in osteoarthritis (Non-Patent Document No. 2: Osteoarthritis and Cartilage, 2001, 9, 641-653). Also, genes the expression levels of which vary upon PMA, IL-1β, or TNFa treatment in the synovial cells and in the chondrocytes of a patient with one of bone and/or joint diseases, i.e., chronic rheumatism, are detected (Non-Patent Document No. 3: Proc. Natl. Acad. Sci. U.S.A., 1997, 94, 2150-2155). Separately, acquisition of disease-associated genes is attempted via comparison of gene expression levels between normal tissues and pathologic tissues with the use of animal models of disease. For example, collagen-induced arthritis (CIA) models were subjected to analysis of variations in gene expression levels using a DNA microarray, and it was reported that the level of gene expression induced was two times or more in 333 among 8,734 cDNAs and that the level of gene expression inhibited was two times or more in 55 cDNAs (Non-Patent Document No. 4: Clin. Immuno., 2002, 105, 155-168). Compared with normal tissues, however, expression levels of numerous genes can vary in pathologic tissues. Accordingly, it is deemed difficult to identify genes that play a key role in the advancement of disease via simple comparison between pathologic tissues and normal tissues.

In order to more directly identify the genes that play a key role in the advancement of disease, identification of genes the expressions of which are induced by a factor that is deeply involved in disease, such as an inflammatory cytokine, is attempted by DNA microarray analysis. For example, the inflammatory cytokine interleukin-1 is allowed to act on a chondrocyte cell line, and the genes induced thereupon are then identified (Non-Patent Document No. 5: Arthritis Res., 2001, 3, 381-388). Since an inflammatory cytokine also has enormous numbers of functions, this technique by itself cannot successfully identify the genes that play a key role in inflammation or disease.

Runx2/Cbfa1 is referred to as a polyoma enhancer-binding protein (PEBP2αA), which is a transcription factor of the gene family having the runt region (Non-Patent Document No. 6: Proc. Natl. Acad. Sci. U.S.A., 1993, 90, 6859-6863). Runx2/Cbfa1 forms a heterodimer with a conjugated transcription factor CbfB/PEBP2β, and it was verified in vitro that it enhances the DNA-binding activity (Non-Patent Document No. 7: Virology, 1993, 194, 314-331; and Non-Patent Document No. 8: Mol. Cell. Biol., 1993, 13, 3324-3339). Runx2/Cbfa1 knockout mice are known to die of respiratory failure shortly after birth and to cause complete anosteoplasia. Runx2/Cbfa1 has been found to be a transcription factor that is essential for bone differentiation (Patent Document No. 1: JP Patent Publication (Unexamined) No. 10-309148 (1998); and Non-Patent Document No. 9: Cell, 1997, 89, 755-764). Further, chondrocyte maturation is inhibited in Runx2/Cbfa1 knockout mice, and expression of the type II Runx2/Cbfa1 isoform is observed in the region from prehypertrophic chondrocytes to hypertrophic chondrocytes in the murine chondrocyte cell line (ATDC5). This indicates that Runx2/Cbfa1 plays a key role in the process of cartilage differentiation and particularly in the process from proliferated cartilage to hypertrophic cartilage (Non-Patent Document No. 10: J. Biol. Chem., 2000, 275, 8695-8702).

It is demonstrated that the expression level of markers of cartilage differentiation, which are observed in the growth cartilages such as type X collagen or osteopontin, is enhanced in the cartilage of a patient of osteoarthritis (Non-Patent Document No. 11: Arthritis Rheum, 1992, 35, 806-811; and Non-Patent Document No. 12: Matrix Biology, 2000, 19, 245-255). Also, calcification is actually observed in the cartilage of a patient of osteoarthritis (Non-Patent Document No. 13: Hone to nankotsu no baioroji (Biology of bones and cartilages), Kanehara & Co., Ltd., 2002). A. Robin Poole describes in the column in the Arthritis & Rheumatism that cartilage differentiation advances and hypertrophic chondrocytes are observed upon cartilage destruction in osteoarthritis, and this process is similar to enchondral ossification that takes place in the growth plates (Non-Patent Document No. 14: Arthritis Rheum., 2002, 46, 2549-2552). It is implied that conversion of permanent cartilages into growth cartilages observed in such osteoarthritis is deeply involved in the advancement of osteoarthritis (Non-Patent Document No. 15: Hiroyasu Iwamoto, Nankotsu soshiki keisei no seigyo kikou (Regulation mechanism for cartilage tissue generation), the academic-award-winning article of the 17^{th} Annual Meeting of the Japanese Society for Bone and Mineral Research).

When Runx2/Cbfa1 was expressed specifically in cartilage with the use of a type II collagen promoter, conversion of permanent cartilages into growth cartilages observed in osteoarthritis was also observed (Non-Patent Document No. 16: J. Cell Biol., 2001, 153, 87-99). This indicates that Runx2/Cbfa1 plays a key role in the advancement of osteoarthritis conditions. Runx2/Cbfa1, however, affects not only cartilage differentiation but also bone differentiation to a great extent. Also, influence of Runx2/Cbfa1 on the immune system is implied. Accordingly, a medicine that targets the direct inhibition of Runx2/Cbfa1 functions may cause side effects. Thus, Runx2/Cbfa1-regulated genes are searched for, and genes having more disease-specific functions are searched for. A Runx2/Cbfa1-regulated gene, Collagenase-3 (MMP-13) (Non-Patent Document No. 17: Mol. Cell Biol., 1999, 19, 4431-4442), has been proven to play the most important role in cartilage destruction in osteoarthritis among the 3 known collagenase species (Non-Patent Document No. 18: J. Clin. Invest., 1996, 97, 761-768). Transgenic mice that exhibit cartilage-specific expression of Collagenase 3 exhibit osteoarthritis-like symptoms, and expression of type X collagen, which is a marker of hypertrophic cartilage differentiation, is also observed (Non-Patent Document No. 19: J. Clin. Invest., 2001, 107, 35-44). Such findings indicate that some of the genes regarding which expression is induced or inhibited by Runx2/Cbfa1 (i.e., the downstream genes of Runx2/Cbfa1) are deeply associated with osteoarthritis. However, thorough analysis of the downstream genes of Runx2/Cbfa1has not been attempted in the past.

The genes that are induced to express upon the forced expression of the transcription factors of interest in the cells have been analyzed. For example, an NF-kB subunit p65, which is a transcription factor deeply associated with inflammation, is forcedly expressed, the expression is analyzed using DNA microarrays, and the genes regarding which expression is induced or inhibited are identified (Non-Patent Document No. 20: Am J Physiol Cell Physiol, 2002, 283, C58-C65). This technique is effective when identifying a gene related to a transcription factor with well-known functions. However, a host cell has endogenous NF-kB, which induces constitutive expression of the gene. Thus, it may be difficult to detect all the NF-kB-regulated genes with high sensitivity. A method that can overcome such difficulty is deemed necessary, although such method has not yet been developed.

WISP-2, which was found to be a downstream gene of Runx2/Cbfa1 with the use of the present invention, is a growth factor that belongs to the connective tissue growth factor/cysteine-rich 61/neuroblastoma overexpressed (CCN) family (Non-Patent Document No. 21: Proc. Natl. Acad. Sci. U.S.A., 1998, 95, 14717-14722). It is known that the WISP-2 expression is enhanced by Wnt-1 and such that expression is attenuated by 2 to 30 times in human colon cancer (Non-Patent Document No. 21: Proc. Natl. Acad. Sci. U.S.A., 1998, 95, 14717-14722). The expression thereof is increased in MCF-7 human mammary cancer cells compared with WISP-2 normal human mammary epithelial cells, and involvement thereof in proliferation of cancer cells is implied (Non-Patent Document No. 22: Biochem. Biophys. Res. Commun., 2001, 282, 421-425). Further, the expression levels thereof were found to be high in osteoblasts and in chondrocytes by in situ hybridization. This indicates that WISP-2 is a control factor of osteoblast functions (Non-Patent Document No. 23: J. Biol. Chem., 1999, 274, 17123-17131), although there has been no report that WISP-2 is regulated by Runx2/Cbfa1 up to the present.

Nopp140, which was also found to be a downstream gene of Runx2/Cbfa1 with the use of the present invention, is the first identified nuclear localization signal-binding protein (140 kDa) (Non-Patent Document No. 24: J. Cell Biol., 1990, 111, 2235-2245). The polypeptide of the Nopp140 gene comprises 699 amino acid residues and has the molecular weight of 130 to 140 kd (95 kd upon dephosphorylation). The Nopp140 protein is a highly phosphorylated protein, and phosphorylation is required in order to achieve functions such as regulation of transcription factor. Such protein has the GTPase/ATPase domain.

In the early days, Nopp140 was considered to function as a chaperone for transporting RNA from the nucleus (Non-Patent Document No. 25: Cell, 1992, 70, 127-138). In a later report, however, it is demonstrated that Nopp140 binds specifically to the alpha-1 acid glycoprotein/enhancer-binding protein (AGP/EBP) and functions as a transcription activator (Non-Patent Document No. 26: Mol. Cell. Biol., 1997, 17, 230-239). Functions of the Nopp140 protein include: regulation of cell cycle (Non-Patent Document No. 27: J Cell Sci., May, 1995, p. 108); regulation of cell viability (Non-Patent Document No. 28: J Cell Sci., May, 1995, 108, Pt. 5, pp. 1911-1920); luteinization and oocyte maturation (Non-Patent Document No. 29: J Mol Endocrinol., December, 2000, 25(3), 275-286); nucleolus formation in the nucleus (Non-Patent Document No. 30: Mol. Biol. Cell, 2000, 11, 567-577); regulation of transcription factors (Non-Patent Document No. 31: Mol. Cell. Biol., 1996, 16, pp. 4257-4263); regulation of rRNA synthesis (Non-Patent Document No. 32: Mol. Cell. Biol., 1999, 19, pp. 8536-8546); chaperon activity and the induction of al acute phase proteins in the acute inflammatory reaction in the liver (Non-Patent Document No. 33: Mol. Cell. Biol., 1997, 17, pp. 230-239); and the increased expression of Nopp140 at the time of neurulation of a chicken (Non-Patent Document No. 34: Development, 2002, 129, pp. 5731-5741). The expression level of the Nopp140 protein is increased in the visceral fat of an obese OLETF rat (Non-Patent Document No. 35: J. Lipid Res., 2000, 41, pp. 1615-1622). The Nopp140 gene is structurally similar to the causative gene (TCOF1) for Treacher Collins syndrome (i.e., craniofacial hypoplasia) (Non-Patent Document No. 36: Mol Biol Cell, September, 2000, 11 (9), pp. 3061-3071). Even though the reports as mentioned above have been made, the role of Nopp140 in cartilage differentiation has not yet been elucidated, and there is no report that Nopp140 is regulated by Runx2/Cbfa1. At present, the correlation between Nopp140 and a bone and/or joint disease is not yet known.

Tem8, which was also found to be a downstream gene of Runx2/Cbfa1 with the use of the present invention, encodes a polypeptide, which is a transmembrane protein comprising 562 amino acid residues, and it serves as a marker in the vascular endothelial cell that is invasive to tumors (Non-Patent Document No. 37: Cancer Res., Sep. 15, 2001, 61 (18), pp. 6649-6655). The polypeptide encoded by the Tem8 gene is highly homologous to the capillary morphogenesis protein (Non-Patent Document No. 38: Proc Natl Acad Sci U.S.A., Apr. 29, 2003, 100 (9), pp. 5170-5174, Epub. Apr. 16, 2003).

The correlation between Tem8 and a bone and/or joint disease has not yet been elucidated. The Tem8 protein is known to act as an anthrax toxin receptor (ATRI) (Non-Patent Document No. 39: Biochem Pharmacol., Feb. 1, 2003, 65 (3), 309-314), and it is also known to bind to the C5 domain of collagen (IV (Non-Patent Document No. 40: Cancer Res., Feb. 1, 2004, 64 (3), pp. 817-820).

GALNT3, which was also found to be a downstream gene of Runx2/Cbfa1 with the use of the present invention, encodes a polypeptide comprising 633 amino acid residues. GALNT3 is highly homologous to other enzymes in the GalNac-glycosyltransferase. family (Non-Patent Document No. 41: J Biol Chem., Sep. 3, 1999, 274 (36), 25362-25370).

The correlation between GALNT3 and a bone and/or joint disease has not yet been elucidated. The GALNT3 protein is a Golgi membrane enzyme, is a GalNac-glycosyltransferase, and is often expressed in the pancreas or in the glandular system, such as in the thyroid gland. Certain types of mutated genes of GALNT3 can cause familial tumoral calcinosis (Non-Patent Document No. 42: Nature Genetics, 2004, 36, pp. 579-581). It is also known that the GALNT3 protein is expressed in gastric or pancreatic cancer cells (Non-Patent Document No. 43: Cancer Sci. January, 2003, 94 (1), pp. 32-36).

HCK, which was also found to be a downstream gene of Runx2/Cbfa1 with the use of the present invention, encodes a polypeptide comprising 526 amino acid residues, is hematopoietic cell kinase, and is expressed in hematocytes. The HCK protein belongs to the Src tyrosine kinase family (Non-Patent Document No. 44: Int J Biochem Cell Biol., June, 1995, 27 (6), pp. 551-63). HCK has the SH2/SH3 domain and is activated upon phosphorylation. It is also activated by mercuric chloride (Non-Patent Document No. 45: Eur. J. Biochem., December, 2000, 267 (24), pp. 7201-8).

The correlation between HCK and a bone and/or joint disease has not yet been elucidated. The HCK protein is an important signaling factor in cell proliferation or immune responses, and it is activated by signals from gp130 (a signaling component of IL-6 or LIFR) (Non-Patent Document No. 46: Mol Cell Biol., December, 2001, 21 (23), pp. 8068-81). The HCK protein phosphorylates Ras GAP or STAT5 and it is involved in signal transduction of an immunoglobulin receptor (Non-Patent Document No. 47: J Biol Chem., Jun. 16, 1995, 270 (24), 14718-24; and Non-Patent Document No. 48: EMBO J., Nov. 1, 2002, 21 (21), pp. 5766-74). The expression of the HCK protein is enhanced and/or activated by imatinib mesylate in the LAMA84 cells (Non-Patent Document No. 49: Blood, Jul. 15, 2004, 104 (2), pp. 509-18, Epub Mar. 23, 2004). The HCK protein allows the proliferation of myeloma cells (Non-Patent Document No. 50: Exp Hematol., December, 1997, 25 (13), pp. 1367-77).

### DISCLOSURE OF THE INVENTION

It is an object of the present invention to discover genes having a novel function of regulating cartilage differentiation and to use such genes for diagnosing, regulating, and treating a bone and/or joint disease such as osteoarthritis. Such genes can be obtained by focusing on the Runx2/Cbfa1 transcription factor deeply associated with a bone and/or joint disease, i.e., osteoarthritis, and introducing Runx2/Cbfa1 into Runx2/Cbfa1-deficient mouse-derived primary chondrocytes or Runx2/Cba1- and p53-deficient mouse-derived chondrocyte cell lines to identify the genes the expressions of which are thereby induced. Further, an object of the present invention is to provide polypeptides encoded by the aforementioned genes, antibodies against such polypeptides, transgenic animals for such genes, and animal models of a bone and/or joint disease (preferably osteoarthritis). It is another object of the present invention to screen for compounds that regulate the functions or expression of the aforementioned polypeptides using the aforementioned objects. Furthermore, an object of the present invention is to provide compounds selected by screening, a means for diagnosing diseases and therapeutic agents using the same.

The first aspect of the present invention to thereby overcome such drawbacks provides a method for obtaining a disease-associated gene, wherein a disease-associated transcription factor is expressed in a cell line that is deficient in such transcription factor or in a primary cultured cell, and the gene the expression of which is thereby induced or inhibited is screened for via subtraction, DNA chip analysis, or via other means to obtain such a disease-associated gene. Preferably, Runx2/Cbfa1 is expressed in a Runxs/Cbfa1- and p53-deficient chondrocyte cell line or in a Runx2/Cbfa1-deficient mouse-derived primary chondrocyte cell line, and the gene expression of which is thereby induced is screened for via subtraction, DNA chip analysis, or via other means to obtain a disease-associated gene concerning Runx2/Cbfa1. More specifically, the present invention provides a method for searching for a disease-associated gene with a lower background level and higher detection sensitivity compared with conventional techniques by introducing a disease-associated transcription factor into a cell that is deficient in such transcription factor. For example, Runx2/Cbfa1 is expressed in a Runx2/Cbfa1-deficient chondrocyte cell line or in a Runx2/Cbfa1-deficient primary cultured cell, and the gene expression of which is thereby induced or inhibited is screened for as a gene associated with regulation of cartilage differentiation.

The second aspect of the present invention provides primary chondrocytes derived from a Runx2/Cbfa1-deficient mouse or cultured chondrocytes and chondrocytes derived from a Runx2/Cbfa1- and p53-deficient mouse that are employed in the first aspect of the present invention. More specifically, the method for obtaining genes using such cells according to the first aspect of the present invention does not induce constitutive gene expression by endogenous Cbfa1 because of deficiency in Cbfa1 transcription factors and can provide a system for inducing and/or inhibiting gene expression with lower background levels and higher detection sensitivity compared with conventional techniques. The Runx2/Cbfa1- and p53-deficient mouse-derived chondrocyte cell lines that can be preferably used in the present invention are deposited as the RU-1 and RU-22 cell lines at the International Patent Organism Depositary of the National Institute of Advanced Industrial Science and Technology (Tsukuba Central 6, 1-1-1 Higashi, Tsukuba, Ibaraki, 305-8566, Japan) under the Budapest Treaty as of Aug. 5, 2003, under the accession numbers FERM BP-10137 (the RU-1 cell line) and FERM BP-10138 (the RU-22 cell line) (the original deposit).

The third aspect of the present invention provides polynucleotides of the genes the expressions of which are induced upon forced expression of Cbfa1 with the use of the cells or cell lines according to the second aspect of the present invention. The fact that the expressions of these genes are induced by Runx2/Cbfa1 is reconfirmed via real-time PCR analysis. Also, the expressions of these genes are inhibited in the Runx2/Cbfa1-deficient murine embryonic skeleton to a greater extent than in the wild-type murine embryonic skeleton. More specifically, the present invention provides polynucleotides having the nucleotide sequence shown in SEQ ID NO: 1, 3, 5, 7, 9, 11, 13, 15, 17, 19, 21, 23, or 25 and exhibiting the expression induced via Runx2/Cbfa1 expression.

Preferably, the present invention provides a transgenic mouse exhibiting cartilage-specific expression under the control of a type II collagen promoter, wherein such transgenic mouse comprises a polynucleotide of a gene encoding a protein capable of stimulating cartilage differentiation and having the nucleotide sequence shown in SEQ ID NO: 5, a polynucleotide of a gene encoding a protein capable of inhibiting cartilage differentiation and having the nucleotide sequence shown in SEQ ID NO: 3, a polynucleotide of a gene encoding a protein capable of stimulating cartilage differentiation and having the nucleotide sequence shown in SEQ ID NO: 1, a polynucleotide of a gene encoding a protein capable of inhibiting cartilage differentiation and having the nucleotide sequence shown in SEQ ID NO: 15, and a polynucleotide of a gene encoding a protein capable of inhibiting chondrogenesis and having the nucleotide sequence shown in SEQ ID NO: 25. Further, the present invention provides a polynucleotide encoding a gene having a novel nucleotide sequence shown in SEQ ID NO: 9.

The aforementioned downstream gene of Runx2/Cbfa1 is derived from a mouse. By searching the database, a human homologue thereof can be easily identified, and it can be easily deduced that such human homologue has similar effects concerning cartilage differentiation. Based on such genetic information, homologous genes can be easily obtained by hybridization. Thus, it can be easily deduced that the resulting gene has similar functions concerning cartilages. Accordingly, the present invention also provides a human-derived polynucleotide, which is homologous to the aforementioned mouse-derived polynucleotide having the nucleotide sequence shown in SEQ ID NO: 27, 29, 31, 33, 35, 37, 39, 41, 43, 45, 47, 49, or 51.

Further, the present invention provides a polynucleotide that has 65% or more homology to the polypeptide encoded by the polynucleotide having the nucleotide sequence shown in SEQ ID NO: 1, 3, 5, 7, 9, 11, 13, 15, 17, 19, 21, 23, or 25 and that encodes a protein capable of stimulating or inhibiting cartilage differentiation. Furthermore, the present invention provides a polynucleotide that is capable of hybridizing under stringent conditions to a polynucleotide having the nucleotide sequence shown in SEQ ID NO: 1, 3, 5, 7, 9, 11, 13, 15, 17, 19, 21, 23, or 25 or a complementary strand thereof and that encodes a protein capable of stimulating or inhibiting cartilage differentiation.

The present invention provides: a recombinant DNA vector comprising the aforementioned polynucleotide or a complementary strand thereof; a transformant transformed with the recombinant DNA vector; a polypeptide encoded by the aforementioned polynucleotide (i.e., a polypeptide comprising an amino acid sequence shown in SEQ ID NO: 2, 4, 6, 8, 10, 12, 14, 16, 18, 20, 22, 24, 26, 28, 30, 32, 34, 36, 38, 40, 42, 44, 46, 48, 50, or 52; a polypeptide comprising an amino acid sequence derived from the aforementioned amino acid sequence by deletion, substitution, or addition of one or several amino acid residues and capable of stimulating or inhibiting cartilage differentiation; or a polypeptide comprising an amino acid sequence having at least 65% homology to the aforementioned amino acid sequence and capable of stimulating or inhibiting cartilage differentiation); an antisense polynucleotide which regulates the expression of the gene consisting of the aforementioned polynucleotide; an RNAi molecule which regulates the expression of the gene consisting of the aforementioned polynucleotide; and an antibody against the aforementioned polypeptide.

The fourth aspect of the present invention provides a method for screening for a therapeutic agent and/or prophylactic agent for a bone and/or joint disease. The screening method according to the present invention involves the use of the aforementioned polynucleotide (i.e., the downstream gene of Runx2/Cbfa1) or a protein encoded by such polynucleotide. This method can provide a compound that regulates the expression of the downstream gene of Runx2/Cbfa1 in vitro and in vivo and a compound that regulates activity of a protein encoded by the downstream gene of Runx2/Cbfa1.

The downstream gene of Runx2/Cbfa1, which had been found to inhibit cartilage differentiation by the aforementioned method, can inhibit the stimulated cartilage differentiation observed in a bone and/or joint disease (preferably osteoarthritis) with the use of a polynucleotide of the gene, a polypeptide encoded by such gene, a vector containing the polynucleotide of the gene, a compound that activates the gene expression or protein functions, or the like. Thus, therapeutic and/or prophylactic effects on a bone and/or joint disease can be expected. In contrast, the downstream gene of Runx2/Cbfa1, which had been found to stimulate cartilage differentiation, can inhibit the stimulated cartilage differentiation observed in a bone and/or joint disease (preferably osteoarthritis) with the use of a compound that inhibits the protein functions, a compound that inhibits the gene expression, an antibody, an RNAi molecule, an antisense polynucleotide, or the like. Thus, therapeutic and/or prophylactic effects on a bone and/or joint disease can be expected.

The disease-associated gene that was screened for by the aforementioned method, for example, the downstream gene of Runx2/Cbfa1 that was found to inhibit or stimulate cartilage differentiation, may be analyzed with the use of the polynucleotide of the gene, the polypeptide encoded by such gene, a vector containing the polynucleotide of the gene, a compound that activates gene expression or protein function, or the like. Thus, diseases (preferably a bone and/or joint disease such as osteoarthritis) can be diagnosed.

The fifth aspect of the present invention provides a transgenic animal associated with the downstream gene of Runx2/Cbfa1, i.e., a transgenic animal model of a bone and/or joint disease with an increased or decreased expression level of the downstream gene of Runx2/Cbfa1. Preferably, the present invention provides a transgenic mouse exhibiting cartilage-specific expression of the downstream gene of Runx2/Cbfa1 under the control of a type II collagen promoter. A transgenic mouse having the downstream gene of Runx2/Cbfa1 capable of stimulating cartilage differentiation may have a phenotype similar to that of osteoarthritis. This can provide a useful animal model of osteoarthritis. Such disease model can effect a method for screening for a candidate pharmaceutical compound and it also serves as a useful tool for analyzing diseases. The present invention also provides a method for producing animal models for a bone and/or joint disease (preferably osteoarthritis) by administration of, for example, a DNA vector containing the polynucleotide of the downstream gene of Runx2/Cbfa1 or a complementary strand thereof, a transformant transformed with such DNA vector, a protein itself encoded by the polynucleotide, an antisense polynucleotide, an RNAi molecule, an antibody, or a compound selected by the aforementioned screening method.

### BRIEF DESCRIPTION OF THE DRAWINGS

FIG. 1 is an optical phase-contrast microscope photograph (×100) showing the morphologies of the Runx2/Cbfa1- and p53-deficient cell lines and the Runx2/Cbfa1-deficient mouse-derived primary cultured chondrocyte of the present invention. RU-1 and RU-22 represent Runx2/Cbfa1- and p53-deficient cell lines.

FIG. 2A and B show the results of analyzing the expression of type II collagen (A) and that of type X collagen (B) in the Runx2/Cbfa1- and p53-deficient cell lines of the present invention (RU-1 and RU-22) via real-time PCR analysis. These cell lines were cultured to be confluent, and total RNAs were extracted. Thereafter, cDNA was synthesized, and the resulting cDNA was used as a template to analyze the gene expression via real-time PCR analysis. The GAPDH expression levels in samples were simultaneously measured, and the expression levels were expressed as the relative value when the GAPDH level was determined to be 1,000.

FIG. 3A and 3B show the results of analyzing the expressions of the Cbfa1 genes and of the cartilage differentiation-associated marker genes upon forced expression of Runx2/Cbfa1 in the Runx2/Cbfa1- and p53-deficient cell lines of the present invention (RU-1) with the use of an adenovirus (A: Cbfa1, PTH/PTHrPR, and type X collagen; B: MMP13, ALP, and BSP). Changes in the gene expression levels in the presence and in the absence of BMP-2 were assayed via real-time PCR analysis over time, from 0 to 15 days after the forced expression of Runx2/Cbfa1.

FIG. 4A and 4B show the results of analyzing the expressions of the cartilage differentiation-associated marker genes and the HNOEL-iso genes upon forced expression of Runx2/Cbfa1 in the Runx2/Cbfa1- and p53-deficient cell lines of the present invention (RU-1) with the use of an adenovirus (A: Ihh, HNOEL-iso, and osteopontin; B: IL11 and osteocalcin). Changes in the gene expression levels in the presence and in the absence of BMP-2 were assayed via real-time PCR analysis over time, from 0 to 15 days after the forced expression of Runx2/Cbfa1.

FIG. 5A and 5B show the results of analyzing the expressions of the Cbfa1 genes and the cartilage differentiation-associated marker genes upon forced expression of Runx2/Cbfa1 in the Runx2/Cbfa1- and p53-deficient cell lines of the present invention (RU-22) with the use of an adenovirus (A: Cbfa1, PHT/PHTrPR, and type X collagen; B: MMP13, ALP, and BSP). Changes in the gene expression levels in the presence and in the absence of BMP-2 were assayed via real-time PCR analysis over time, from 0 to 15 days after the forced expression of Runx2/Cbfa1.

FIG. 6A and 6B show the results of analyzing the expressions of the cartilage differentiation-associated marker genes and the HNOEL-iso genes upon forced expression of Runx2/Cbfa1 in the Runx2/Cbfa1- and p53-deficient cell lines of the present invention (RU-22) with the use of an adenovirus (A: Jhh and HNOEL-iso; B: IL11, osteocalcin, and osteopontin). Changes in the gene expression levels in the presence and in the absence of BMP-2 were assayed via real-time PCR analysis over time, from 0 to 15 days after the forced expression of Runx2/Cbfa1.

FIG. 7A and 7B show the results of analyzing the expressions of the Cbfa1 genes and the cartilage differentiation-associated marker genes upon forced expression of Runx2/Cbfa1 in the Runx2/Cbfa1-deficient mouse-derived primary cultured chondrocytes of the present invention with the use of an adenovirus (A: Cbfa1, PHT/PHTrPR, and type X collagen; B: MMP13, ALP, and BSP). Changes in the gene expression levels in the presence and in the absence of BMP-2 were assayed via real-time PCR analysis over time, from 0 to 15 days after the forced expression of Runx2/Cbfa1.

FIG. 8A and 8B show the results of analyzing the expressions of the cartilage differentiation-associated marker genes and the HNOEL-iso genes upon forced expression of Runx2/Cbfa1 in the Runx2/Cbfa1-deficient mouse-derived primary cultured chondrocytes of the present invention with the use of an adenovirus (A: Ihh and HNOEL-iso; B: IL11, osteocalcin, and osteopontin). Changes in the gene expression levels in the presence and in the absence of BMP-2 were assayed via real-time PCR analysis over time, from 0 to 15 days after the forced expression of Runx2/Cbfa1.

FIG. 9 shows the results of a DNA microarray analysis of the genes induced to express one day after the forced expression of Runx2/Cbfa1 in the Runx2/Cbfa1-deficient cell lines (RU-1), the p53-deficient cell lines (RU-22), and the Runx2/Cbfa1-deficient mouse-derived primary cultured cell lines, with the use of an adenovirus.

FIG. 10 shows the results of an experiment to reconfirm the Runx2/Cbfa1-induced expressions of the genes, the Runx2/Cbfa1-induced expressions of which had been found via DNA microarray analysis, via real-time PCR analysis. The Runx2/Cbfa1-induced expressions of the Cbfa1, Tem8, and WISP2 genes was reconfirmed with the use of the cell line (RU-1), the expression of which was found to be induced via DNA microarray analysis.

FIG. 11 shows the results of an experiment to reconfirm the Runx2/Cbfa1-induced expressions of the genes, the Runx2/Cbfa1-induced expressions of which had been found via DNA microarray analysis, via real-time PCR analysis. The Runx2/Cbfa1-induced expressions of the Cbfa1, KEST, the MYB binding protein (p160) 1a, and Nopp140 genes was reconfirmed with the use of the cell line (RU-22), the expression of which was found to be induced via DNA microarray analysis.

FIG. 12A to 12C show the results of an experiment to reconfirm the Runx2/Cbfa1-induced expressions of the genes, the Runx2/Cbfa1-induced expressions of which had been found via DNA microarray analysis, via real-time PCR analysis. The Runx2/Cbfa1-induced expressions of the genes were reconfirmed with the use of the primary cultured cell line, the expression of which was found to be induced via DNA microarray analysis.

FIG. 13A and 13B show the results of analysis of the expressions of the genes, the Runx2/Cbfa1-induced expressions of which had been found via DNA microarray analysis, in an embryonic skeleton of a wild-type mouse and in that of a Runx2/Cbfa1-deficient mouse. Total RNAs were extracted from the skeleton of the wild-type mouse (WT) at days 13.5, 15.5, and 18.5 of embryonic development and from a Runx2/Cbfa1-deficient mouse (KO) at day 18.5 thereof, and cDNAs were synthesized. Thereafter, the synthesized cDNAs were used as templates to analyze the expression patterns via real-time PCR (the chondrocyte cell lines).

FIG. 14A to 14C show the results of expression analysis of the genes, the Runx2/Cbfa1-induced expressions of which had been found via DNA microarray analysis, in an embryonic skeleton of a wild-type mouse and in that of a Runx2/Cbfa1-deficient mouse. Total RNAs were extracted from the skeleton of the wild-type mouse (WT) at days 13.5, 15.5, and 18.5 of embryonic development and from a Runx2/Cbfa1-deficient mouse (KO) at day 18.5 thereof, and cDNAs were synthesized. Thereafter, the synthesized cDNAs were used as templates to analyze the expression patterns via real-time PCR (the primary cultured chondrocytes).

FIG. 15 shows photographs of images of HE-stained sections of the lower-body skeletons of a Wisp2 transgenic mouse (HE) and images showing the results of expression analysis via in situ hybridization of type II collagen (Col2al), a PTH receptor (Pthrl), type X collagen (Col10al), and osteopontin. These photographs indicate that cartilage differentiation is delayed in the Wisp2 transgenic mouse (Wisp2 tg) compared with the wild-type mouse (Wt).

FIG. 16A and B show photographs of images of the HE-stained lower-body skeleton of a wild-type mouse (A: WT) and that of a Wisp2 transgenic mouse (B: WISP2). These photographs indicate that cartilage differentiation is delayed in the Wisp2 transgenic mouse compared with the wild-type mouse.

FIG. 17A and B show photographs of the appearances of the wild-type mouse (Wt) and of the Nopp140 transgenic mouse (Nopp140 tg) (A) and photographs of images of the HE-stained tibias thereof (B). These photographs indicate that cartilage differentiation is advanced at a given stage in the Nopp140 transgenic mouse compared with the wild-type mouse.

FIG. 18 shows photographs showing the head appearance of a wild-type mouse and that of the Tem8 transgenic mouse (A) and photographs of images of stained skeletons thereof (B). These photographs indicate that cartilage differentiation is advanced in the Tem8 transgenic mouse (Tem8) compared with the wild-type mouse.

FIG. 19A shows photographs of the appearance of a wild-type mouse and that of the HCK transgenic mouse at day 14.5 of embryonic development (A-(2)) and an image of the stained skeletons thereof (A-(1)).

FIG. 19B and 19C are photographs showing images of HE staining (B-(1), HE); images showing the results of expression analysis via in silu hybridization for the followings: type I collagen (B-(2): Collal), type II collagen (B-(3): Col2al), type X collagen (B-(4): Col10al), osteopontin (B-(5): osteopontin), Indian hedgehog (B-(6): Ihh), a PTH receptor (B-(7): Pthrl), Hck (B-(8)), MMP13 (C-(1)), BSP (C-(2)), and VEGF (C-(3)); and images showing the results of analyzing osteoclasts by TRAP staining (C-(4)). The HCK transgenic mouse had a smaller body size and stubbier limbs, and it exhibited abnormalities in differentiation in cartilage tissues.

FIG. 20 shows photographs of the HE-stained HCK transgenic mouse at day 16.5 of embryonic development (HE) and the results of expression analysis by in situ hybridization for type II collagen (Col2al), a PTH receptor (PthRP), and type X collagen (CollOal). In the tibia of the HCK transgenic mouse, unorganized abnormal growth of cells inhibited the formation of the normal epiphyseal plate, and longitudinal growth was abnormal.

FIG. 21 A to C show photographs of the appearance of the HCK transgenic mouse at day 18.5 of embryonic development (A), the images of stained skeleton thereof (B), and the results of HE staining thereof (C). Compared with a wild-type mouse, the HCK transgenic mouse had a smaller body size, a more protruding abdomen, and stubbier limbs (FIG. 21A). There was a cleavage between the nose and the upper jaw, and the process of fusion between the nose and the upper jaw was inhibited. Skeleton staining revealed that calcified tissue stained with alizarin red was decreased and extracellular matrix stained with alizarin blue was increased (FIG. 21B). The chondrocytes of the mouse were found to be immature based on the HE-stained images, and invasion and proliferation of mesenchymal cells were observed in the vicinity thereof. The enchondral bone had lost its proximal-distal direction and exhibited abnormal morphologies. The growth plates were not organized and joints had adhered to each other (FIG. 21C).

FIG. 22A is a photograph showing the appearance of the HCK transgenic mouse at day 18.5 of embryonic development (A-(1)) and an image of the stained skeleton thereof (A-(2)).

FIG. 22B and 22C are photographs of images of HE- and Kossa-staining (B-(1), HE-Kossa); images showing the results of expression analysis by in situ hybridization for type I collagen (B-(2): Collal), type II collagen (B-(3): Col2al), type X collagen (B-(4): Col10al), osteopontin (B-(5): osteopontin), osteocalcin (B-(6): osteocalcin), a PTH receptor (B-(7): Pthrl), Indian hedgehog (B-(8): Ihh), Hck (C-(1)), MMP13 (C-(2)), and BSP (C-(3)); images showing the results of analyzing osteoclasts by TRAP staining (C-(4)); and images showing the results of analyzing proteoglycan by safranin 0 staining (C-(5)). The number of Col2al-expressing chondrocytes decreased, and bone formation involved mesenchymal cell invasion and proliferation without normal processes.

FIG. 23A and B show the structure of a gene construct for preparing the GALNT3 transgenic mouse (A) and a photograph of the appearance of the GALNT3 transgenic mouse at day 18.5 of embryonic development (B). Compared with a wild-type mouse (wt), the GALNT3 transgenic mouse (Galnt3 tg) had a smaller body size, stubbier limbs, a smaller pectoral region, and a more protruding abdomen.

FIG. 24 is a photograph showing an image of stained skeleton of the GALNT3 transgenic mouse at day 18.5 of embryonic development. Compared with a wild-type mouse, the GALNT3 transgenic mouse (Galnt3 tg) had a smaller pectoral region and the significantly decreased bones calcified upon enchondral ossification.

FIG. 25A and B show photographs of images of HE- and Kossa-stained tibia of the GALNT3 transgenic mouse at day 16.5 of embryonic development (B is a partial enlarged image of A). Angioinvasion was observed in a wild-type mouse (wt); however, it was not observed in the GALNT3 transgenic mouse (Galnt3 tg), and articular cavity formation was incomplete.

FIG. 26 is a photograph showing the results of expression analysis via in situ hybridization for type II collagen (Col2al) in the tibia of the GALNT3 transgenic mouse at day 16.5 of embryonic development. Compared with a wild-type mouse, the GALNT3 transgenic mouse (Galnt3 tg) exhibited abnormal distribution of type II collagen expression.

FIG. 27 is a photograph showing the results of expression analysis via in situ hybridization for type X collagen (CollOal) in the tibia of the GALNT3 transgenic mouse at day 16.5 of embryonic development. Type X collagen expression was observed in hypertrophic cartilage of both the wild-type mouse and the GALNT3 transgenic mouse (GaInt3 tg).

FIG. 28 is a photograph showing the results of expression analysis via in sit hybridization for osteopontin in the tibia of the GALNT3 transgenic mouse at day 16.5 of embryonic development. Osteopontin expression was observed in hypertrophic cartilage of both the wild-type mouse and the GALNT3 transgenic mouse (Galnt3 tg).

FIG. 29 shows photographs of the results of expression analysis via in silu hybridization (A: mRNA) and via immunostaining (B: protein) of aggrecan in the tibia of the GALNT3 transgenic mouse at day 18.5 of embryonic development. The mRNA expression of aggrecan (A) was slightly increased in the GALNT3 transgenic mouse (Galnt3 tg) compared with the wild-type mouse (wt); however, aggrecan expression was decreased at the protein level (B: protein).

FIG. 30 is a photograph showing the results of safranin 0 staining of the tibia of the GALNT3 transgenic mouse at day 16.5 of embryonic development. Compared with a wild-type mouse, the staining affinity of the GALNT3 transgenic mouse (Galnt3 tg) was significantly deteriorated, which indicates significantly decreased proteoglycan content.

FIG. 31A and B show photographs of the results of PAS staining of the anklebone of the GALNT3 transgenic mouse at day 18.5 of embryonic development (B is a partial enlarged image of A). Compared with a wild-type mouse (wt), the staining affinity of the GALNT3 transgenic mouse (Galnt3 tg) was enhanced, which indicates an increased level of mucin-like glycoproteins.

FIG. 32 is a photograph showing an image of immunostained fibronectin in the tibia of the GALNT3 transgenic mouse at day 18.5 of embryonic development. Compared with a wild-type mouse (wt), the distance between adjacent chondrocytes was small in the GALNT3 transgenic mouse (Galnt3 tg), which indicates a lowered amount of extracellular matrix.

FIG. 33A is a photograph showing the results of analyzing, using Brdu labeling, the chondrocyte proliferation in the tibia of the GALNT3 transgenic mouse at day 18.5 of embryonic development. Compared with a wild-type mouse (wt), chondrocyte proliferation was enhanced in the GALNT3 transgenic mouse (Galnt3 tg).

FIG. 33B is a chart showing the number of Brdu-positive cells.

FIG. 34 is a photograph showing the results of analyzing apoptosis in the tibia of the GALNT3 transgenic mouse at day 18.5 of embryonic development by Tunnel staining. Compared with a wild-type mouse (wt), chondrocyte apoptosis was enhanced in the TALNT3 transgenic mouse (Galnt3 tg).

### PREFERRED EMBODIMENTS OF THE INVENTION

Hereafter, the present invention is described in detail. This patent application claims priority from Japanese Patent Application No. 2003-359172 filed on Oct. 20, 2003, and the contents as disclosed in the description and/or drawings thereof are herein incorporated.

[Definition of Bone and/or Joint Disease]

In the present invention, the term “bone and/or joint disease” is a general term for systemic diseases or diseases resulting in primary lesions in joints, one of the symptoms of which is the abnormality in bones and cartilages that form skeletons. More specifically, a bone and/or joint disease can be defined by a clear correlation between bone and cartilage differentiation and lesion development. In the present invention, the bone and/or joint disease is preferably associated with cartilage differentiation. Representative examples of a bone and/or joint disease include osteoarthritis and chronic rheumatism. Also, juvenile rheumatoid arthritis, psoriatic arthritis, Reiter's syndrome, systemic lupus erythematodes (SLE), progressive systemic sclerosis, Charcot's joint (neuropathic arthropathy), CPPD crystal deposition disease, BCP crystal deposition disease, and gout may occasionally involve arthritis as a lesion. In a broad sense, the bone and joint disease includes such diseases. From the viewpoint of the correlation between bone differentiation and lesion, osteoporosis is a bone and/or joint disease in a broad sense.

Osteoarthritis is a disease characterized by degeneration and abration of joint cartilage and stiffening of and proliferative change in the subchondral bones, and secondary synovitis is also observed. Osteoarthritis is considered to be an aging-based multifactorial disease. Besides aging, sexuality (female), obesity, and external injuries (such as ligament or medial meniscus injuries) can be risk factors of osteoarthritis, although causal factors of OA have not yet been clarified. In osteoarthritic joint cartilage, the enhanced expression level of the markers for hypertrophic cartilage and calcification are observed, which are not observed in normal permanent cartilages. This implies that hypertrophied cartilage (stimulated differentiation) is associated with the osteoarthritis development. Accordingly, regulation of cartilage differentiation is considered to improve osteoarthritic symptoms.

[Definition of the Downstream Gene of Runx2/Cbfa1]

In the present invention, a gene the expression of which is induced by Runx2/Cbfa1 is referred to as the downstream gene of Runx2/Cbfa1. The term “downstream gene of Runx2/Cbfa1” refers to any one of or a plurality of arbitrary genes selected from: the gene having the nucleotide sequence shown in SEQ ID NO: 1, 3, 5, 7, 9, 11, 13, 15, 17, 19, 21, 23, or 25; the human homologous gene (the human homologous polynucleotide) having the nucleotide sequence shown in SEQ ID NO: 27, 29, 31, 33, 35, 37, 39, 41, 43, 45, 47, 49, or 51; and a gene having functions equivalent to those of the gene the expression of which is induced by Runx2/Cbfa1 (Table 1). A specific example of the “gene having functions equivalent to those of the gene the expression of which is induced by Runx2/Cbfa1” used herein is a counterpart gene in animal species other than mice and humans. The amino acid sequences of the polypeptides encoded by such genes are shown in SEQ ID NO: 2, 4, 6, 8, 10, 12, 14, 16, 18, 20, 22, 24, 26, 28, 30, 32, 34, 36, 38, 40, 42, 44, 46, 48, 50, or 52. Since the expressions of these genes are induced by Runx2/Cbfa1, these genes may be capable of regulating cartilage differentiation, and they may be associated with diseases or pathological conditions associated with the regulation of cartilage differentiation.

TABLE 1
List of the downstream genes of Runx2/Cbfa1

| SEQ ID NO: | Mouse genes | SEQ ID NO: | Human homologue genes |
|---|---|---|---|
| 1 | Tumor endothelial marker 8 precursor | 27 | Homo sapiens tumor endothelial marker |
| | (Tem8) | | 8 (TEM8), transcript variant 1 |
| 3 | WNT1 inducible signaling pathway | 29 | Homo sapiens WNT1 inducible signaling |
| | protein 2 (Wisp2) | | pathway protein 2 (WISP2) |
| 5 | Nucleolar and coiled-body | 31 | Homo sapiens nucleolar and coiled-body |
| | phosphoprotein 1 (Nolc1) (Nopp140) | | phosphoprotein 1 (NOLC1) |
| 7 | MYB binding protein (P160) 1a | 33 | Homo sapiens MYB binding protein |
| | (Mybbp1a) | | (P160) 1a (MYBBP1A) |
| 9 | DNA segment, Chr 13, Wayne State | 35 | Homo sapiens hypothetical protein |
| | University 123, expressed (k. EST) | | FLJ20303 |
| 11 | RIKEN cDNA 2810002E22 gene | 37 | Homo sapiens HNOEL-iso protein |
| | (HNOEL-iso homolog) | | (HNOEL-iso) |
| 13 | BRP39 | 39 | YKL40 (gp39) Homo sapiens chitinase |
| | | | 3-like 1 (cartilage glycoprotein-39) |
| 15 | Hemopoietic cell kinase (HCK) | 41 | Hemopoietic cell kinase (HCK) |
| 17 | Lysyl oxidase-like 2 (LOXL2) | 43 | Lysyl oxidase-like 2 (LOXL2) |
| 19 | Protein tyrosine phosphatase, | 45 | PTPRF interacting protein |
| | receptor-type, F interacting protein, | | (PPFIBP2) (liprin beta 2) |
| | binding protein 2 (PPFIBP2) | | |
| 21 | WNT1 inducible signaling pathway | 47 | WNT1 inducible signaling pathway |
| | protein 1 (Wisp1) | | protein 1 (WISP1) |
| 23 | Placental growth factor (PIGF) | 49 | Placental growth factor, vascular |
| | | | endothelial growth factor-related protein |
| | | | (PGF) |
| 25 | UDP-N-acetyl-alpha-D-galactosamine: | 51 | UDP-N-acetyl-alpha-D-galactosamine: |
| | polypeptide N-acetylgalactosaminyl | | polypeptide |
| | transferase 3 (GALNT3) | | N-acetylgalactosaminyltransferase 3 |
| | | | (GalNAc-T3) (GALNT3) |

[Genes Associated with Bone and/or Joint Disease]

The present invention relates to a polynucleotide of a gene associated with a bone and/or joint disease. Preferably, the present invention relates to a polynucleotide of a gene the expression of which is induced by Runx2/Cbfa1 and which is capable of stimulating or inhibiting cartilage differentiation. Particularly preferably, the present invention relates to a polynucleotide of a gene associated with osteoarthritis. Specifically, such gene can be identified by introducing Runx2/Cbfa1 into Runx2/Cbfa1-deficient mouse-derived primary chondrocytes or a Runx2/Cbfa1- and p53 -deficient chondrocyte cell line and screening for genes the expressions of which are thereby induced or inhibited, by, for example, subtraction or DNA microarray analysis (a DNA chip).

The Runx2/Cbfa1-deficient mouse-derived primary chondrocytes and the Runx2/Cbfa1- and p53-deficient chondrocyte cell lines can be prepared in the manner as described in the section [Cells and cell lines]. A method for introducing Runx2/Cbfa1into such cells can be carried out in accordance with a gene recombination technique known in the art (i.e., genetic transformation). An example of a more specific procedure is described in Example 2.

The polynucleotides of the genes the expressions of which are induced by Runx2/Cbfa1 identified above (the left column in Table 1) are derived from mice. Human homologues thereof can be easily identified by searching public databases. The nucleotide sequences of the human homologues identified by searching of public databases (e.g., the GenBank database) are shown in SEQ ID NOs: 27, 29, 31, 33, 35, 37, 39, 41, 43, 45, 47, 49, and 51 (the right column in Table 1). Human homologues can also be identified via homology search of public databases. In such a case, human homologues can be identified by selecting genes encoding proteins that are most homologous to the polypeptides encoded by the aforementioned genes, from among the genes encoding proteins having 60% or more, and preferably 65% or more, homology to such polypeptides. Homology search of proteins is known in the art, and homology can be easily determined with the use of, for example, a known protein homology search program, i.e., BLAST (Altschul S. F. et al., 1990, Basic local alignment search tool, J. Mol. Biol. 215: 403-410; and Karlin S. and Altschul S. F., 1990, Proc. Natl. Acad. Sci. U.S.A., 87: 2264-2268).

Further, the aforementioned polynucleotide encoding the gene the expression of which is induced by Runx2/Cbfa1 or a complementary strand thereof is experimentally used as a probe to obtain a polynucleotide that hybridizes under stringent conditions. Thus, a human gene that is highly homologous and functionally equivalent to a mouse-derived gene can be identified, for example. It can be readily deduced that the human homologue thus obtained retains equivalent functions concerning cartilage differentiation. In the present invention, “a polynucleotide that is capable of hybridizing under stringent conditions” can be obtained in accordance with, for example, the method described in Molecular Cloning, 2nd Ed., J. Sambrook et al., 1989. The phrase “capable of hybridizing under stringent conditions” used herein indicates that positive hybridization signals can still be observed when hybridization is carried out by heating in a solution (6×SSC, 0.5% SDS, and 50% formamide) at 42° C., followed by washing in a solution (0.1×SSC and 0.5% SDS) at 68° C., for example.

Whether or not the polynucleotide thus obtained actually encodes a protein capable of stimulating or inhibiting cartilage differentiation can be examined by, for example, the techniques described in Examples 8 and 9. For example, it can be confirmed by expressing the polynucleotide of interest and determining if cartilage differentiation can be thereby stimulated or inhibited.

Some of the polynucleotides encoding the genes the expressions of which are induced by Runx2/Cbfa1, regarding which reproducibility is verified by real-time PCR analysis, and the expressions of which are inhibited in a Runx2/Cbfa1-deficient murine skeleton compared to those in a wild-type murine embryonic skeleton, a novel gene having the nucleotide sequence shown in SEQ ID NO: 9 is found. Concerning this gene, the original sequence of the sequence spotted on the glass of the DNA microarray used for analysis is registered in public databases, although only a part of the sequence is available. In the present invention, the full-length sequence of the novel gene was identified via the RACE method based on the aforementioned partial sequence. Numerous genes, only the partial sequences of which are known, were spotted on the DNA microarray that was used for analysis, and the full-length sequences thereof could be determined via the RACE method. With this technique, accordingly, EST, the expression of which is induced or inhibited by Runx2/Cbfa1, can be identified, and then the full-length genes can be analyzed via the RACE method to identify the novel genes. As with the case of other downstream genes of Runx2/Cbfa1, the expressions of the genes thus identified can be regulated by Runx2/Cbfa1, they are preferably capable of regulating cartilage differentiation, and they are more preferably associated with a bone and/or joint disease such as osteoarthritis.

The polynucleotides having the nucleotide sequences shown in SEQ ID NOs: 3 and 15 are capable of inhibiting cartilage differentiation. In contrast, the polynucleotides having the nucleotide sequences shown in SEQ ID NOs: 5 and 1 are capable of stimulating cartilage differentiation. Further, the polynucleotide having the nucleotide sequence shown in SEQ ID NO: 25 is capable of inhibiting chondrogenesis.

The nucleotide sequence of the polynucleotide of the gene according to the present invention is shown in SEQ ID NO: 1, 3, 5, 7, 9, 11, 13, 15, 17, 19, 21, 23, 25, 27, 29, 31, 33, 35, 37, 39, 41, 43, 45, 47, 49, or 51. Further, the polynucleotide of the present invention may be a DNA sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, or 51, or a DNA sequence derived from the nucleotide sequence registered in a public database by spontaneous or induced mutation, such as deletion, substitution, or addition, of one or several DNAs. Means for conducting deletion, substitution, addition, or insertion of DNA are known in the art. Examples thereof include a method for generating a deletion mutant with the use of exonuclease and a site-directed mutagenesis. Some downstream genes of Runx2/Cbfa1 according to the present invention may have a plurality of isoforms depending on different splicing sites. It can be readily deduced that these isoforms are functionally similar to one another. The downstream genes of Runx2/Cbfa1 of the present invention encompass such gene isoforms. [Cloning of a full-length nucleotide sequence from a partial nucleotide sequence] Once part of the nucleotide sequence of the downstream gene of Runx2/Cbfa1 is elucidated, a person skilled in the art can define the full-length sequence of such gene based on the information concerning partial nucleotide sequence. For example, the full-length nucleotide sequence can be obtained via in silico cloning. Specifically, an enormous quantity of EST information accumulated in public databases is searched to inquire for the nucleotide sequence of EST (the query sequence) that constitutes part of the aforementioned gene. Based on the results of the inquiry, other piece of EST information on the sequence having the nucleotide sequence consistent with the query sequence over a given length is obtained. The other obtained piece of EST information is used as a new query sequence to repeatedly obtain other pieces of EST information. By the repeat of this procedure, a plurality of EST sets that share partial nucleotide sequences can be obtained. Such EST sets are referred to as a cluster. The EST nucleotide sequences constituting a cluster are combined and integrated into a nucleotide sequence. Thus, the nucleotide sequence of the gene of interest can be elucidated.

Further, a person skilled in the art can design primers for PCR based on the nucleotide sequence determined via in silico cloning. If the amplification of a gene fragment having the length as designed is confirmed by RT-PCR using such primers, this can be used to verify the actual existence of the gene consisting of the determined nucleotide sequence.

Also, the results of in silico cloning can be evaluated by Northern blotting. Northern blotting is carried out using the probes designed based on the determined nucleotide sequence information. If a band that is consistent with the aforementioned nucleotide sequence information is consequently detected, the existence of the gene having the determined nucleotide sequence can be verified.

In addition to in silico cloning, a gene of interest can be experimentally isolated. At the outset, a cDNA clone having the nucleotide sequence information registered as EST is obtained, and the full-length nucleotide sequence comprising cDNA of such clone is determined. This can consequently result in elucidation of the full-length sequence of cDNA. This procedure can at least result in the elucidation of a longer nucleotide sequence. The length of the cDNA of the clone can be experimentally confirmed in advance if the vector structure is known.

Even if the clone with the nucleotide sequence information of EST is not available, a portion of the gene with an unknown nucleotide sequence can be obtained based on the partial nucleotide sequence. Such procedure is known in the art. For example, a cDNA library may be screened using the EST probe, and this procedure can occasionally elucidate a longer nucleotide sequence. With the use of cDNA library containing a large quantity of full-length cDNAs, full-length clones can be easily isolated. For example, the cDNA library synthesized based on the principle of oligo-capping is considered to contain a large quantity of full-length cDNAs.

Also, a method for synthesizing a region of the gene containing an unknown nucleotide sequence based on the information concerning the partial nucleotide sequence is known. For example, the RACE method is a representative technique for isolating a gene containing an unknown nucleotide sequence. In the RACE method, an oligonucleotide linker is artificially ligated to the end of cDNA. The nucleotide sequence of such oligonucleotide linker is known in advance. Accordingly, primers for PCR can be designed based on a region with a known nucleotide sequence as EST and the nucleotide sequence information of the oligonucleotide linker. A region with an unknown nucleotide sequence can be specifically synthesized by PCR using the primers thus designed.

If a full-length sequence information is obtained by the RACE method, cDNA library cloning, in silico cloning using public databases, or other means, a person skilled in the art can prepare synthetic DNA based on the full-length sequence information and bind the synthetic DNA to obtain a polynucleotide having the full-length sequence. Via the method involving the use of synthetic DNA, mutation can be introduced into any site. Thus, a mutant comprising substitution, deletion, or addition of one or several amino acid residues can be prepared while maintaining the functions relevant to cartilage differentiation. The polynucleotide thus obtained has functions equivalent to those of a polynucleotide having the same sequence obtained via another technique, and it can be used in the same manner.

[Gene Polymorphism]

In general, the genes of higher order animals frequently involve polymorphisms. Also, a number of molecules exist, which generate isoforms consisting of alternating amino acid sequences in the process of splicing. The downstream gene of Runx2/Cbfa1 according to the present invention includes genes having activities similar to those of the downstream gene of Runx2/Cbfa1, even though such genes have different nucleotide sequences due to polymorphisms or isoforms.

[Homologues in Different Species]

In the present invention, the downstream gene of Runx2/Cbfa1 is not limited to that of the mouse or human homologues, and it includes counterparts in other animal species. Accordingly, the downstream gene of Runx2/Cbfa1 of a species other than mice and humans refers to a homologue of the downstream gene of Runx2/Cbfa1 intrinsic to the species of interest or an extrinsic downstream gene of Runx2/Cbfa1, which has been introduced into the organism, unless otherwise specified.

In the present invention, the term “homologue of mouse downstream gene of Runx2/Cbfa1” refers to a gene derived from a species other than a mouse that can hybridize under stringent conditions using the murine gene as the probe and that has functions equivalent to those of the murine downstream gene of Runx2/Cbfa1. The phrase “that hybridizes under stringent conditions” used herein indicates that positive hybridization signals can still be observed when hybridization is carried out by heating in a solution (6×SSC, 0.5% SDS, and 50% formamide) at 42° C., followed by washing in a solution (0.1×SSC and 0.5% SDS) at 68° C., for example. The temperature conditions for hybridization or washing that significantly affect stringency can be modified in accordance with the melting temperature (Tm). Tm varies depending on the proportion of constitutive nucleotides in relation to the hybridizing nucleotide pairs or the composition of the solution for hybridization (a salt concentration, a formamide concentration, a sodium dodecyl sulfate concentration, or the like). A person skilled in the art accordingly takes such conditions into consideration, he can test for the conditions that can yield equivalent stringency, or he can empirically determine such conditions. The term “gene having functions equivalent to those of the downstream gene of Runx2/Cbfa1” used herein refers to a gene encoding a protein having activity or function identical or similar to a protein the expression of which is inhibited or enhanced by the downstream gene of Runx2/Cbfa1 of a mouse or of another species, which is encoded by the downstream gene of Runx2/Cbfa1 of a mouse.

The human homologue gene of the murine downstream gene of Runx2/Cbfa1 was searched for in public databases. As a result, a human homologue exhibiting 65% or more homology in terms of the amino acid sequence of the protein was identified. When the homologues of animal species other than humans are searched for in public databases based on mouse sequences, accordingly, the searched-for gene may be a functionally similar counterpart gene, as long as it exhibits 65% or more homology at the amino acid sequence level.

[Primer or Probe]

A primer or probe can be a polynucleotide comprising the whole or a part of the nucleotide sequence of the downstream gene of Runx2/Cbfa1 or a polynucleotide comprising a nucleotide sequence of at least 15 nucleotides complementary to the complementary strand of the former polynucleotide. The term “complementary strand” used herein refers to one strand to the other strand of double-stranded DNA consisting of base pairs of A:T (U in the case of RNA) or G:C. The “complementary” strands are not necessarily completely complementary in a region comprising at least 15 continuous nucleotides. These strands may exhibit 70% or more, preferably 80% or more, more preferably 90% or more, and further preferably 95% or more homology to each other. Nucleotide sequence homology can be determined using an algorithm such as BLAST.

Such polynucleotide can be used as a probe for detecting the downstream gene of Runx2/Cbfa1 or as a primer for amplifying the downstream gene of Runx2/Cbfa1. A polynucleotide primer generally has a length of 15 bp to 100 bp, and preferably 15 bp to 35 bp. A polynucleotide probe has at least part of or the entire sequence of the downstream gene of Runx2/Cbfa1 (or a complementary strand thereof), and a DNA sequence of at least 15 bp is used. When the polynucleotide is used as a primer, the 3′ region needs to be complementary, and a restriction enzyme recognition sequence, a tag, or the like can be added to the 5′ end.

In the present invention, the “polynucleotide” may be DNA or RNA. Such polynucleotide may be synthetic or naturally occurring. Probe DNA used for hybridization is generally labeled. Examples of labeling methods include the following:

1) labeling by nick translation with the use of DNA polymerase I;

2) terminal labeling with the use of polynucleotide kinase;

3) fill-in labeling of the terminus with the use of the Klenow fragment (Berger S L, Kimmel A R., 1987, Guide to Molecular Cloning Techniques, Methods in Enzymology, Academic Press; Hames B D, Higgins S. J., 1985, Genes Probes: A Practical Approach, IRL Press; Sambrook J, Fritsch E F, Maniatis T., 1989, Molecular Cloning: a laboratory Manual, 2nd Ed. Cold Spring Harbor Laboratory Press);

4) labeling by transcription with the use of RNA polymerase (Melton D A, Krieg P A, Rebagkiati M R, Maniatis T, Zinn K, Green M R. Nucleic Acid Res., 1984, 12, 7035-7056); and

5) incorporation of modified nucleotides into DNA without the use of a radioisotope (Kricka L. J., 1992, Nonisotropic DNA Probing Techniques, Academic Press).

Among polynucleotides, oligonucleotides have a relatively low degree of polymerization, and polynucleotides include oligonucleotides.

[Techniques for Searching for Genes]

The present invention relates to a technique for searching for a disease-associated gene by introducing a disease-associated transcription factor into a cell that is deficient in such transcription factor and then allowing the transcription factor to be expressed. Preferably, the present invention further relates to a technique for searching for a gene by introducing Runx2/Cbfa1 into a primary chondrocyte or chondrocyte cell line that is deficient in Runx2/Cbfa1 and then allowing the transcription factor to be expressed. This technique can provide a method for searching for a gene without constitutive gene expression caused by endogenous genes and with a very low background level. This may enable the detection of genes that could not be detected in the past. An example of a disease-associated transcription factor is a transcription factor associated with inflammation, such as NF-kB. Disease-associated transcription factors may be induced or inhibited under pathologic conditions or may be induced in accordance with the progress of disease.

A cell that is deficient in a transcription factor may be of an established cell line, or it can be prepared by genetic engineering based on the genetic information of the transcription factor. Alternatively, a cell that is deficient in a transcription factor can be obtained via various types of chemical treatment.

A transcription factor may be introduced into a cell by a transformation technique known in the art. The transcription factor is introduced into the cell, the expression thereof is confirmed, and the gene the expression of which is induced or inhibited is screened for via subtraction, DNA chip analysis, or via other means.

[Cells or Cell Lines]

The present invention relates to the Runx2/Cbfa1-deficient mouse-derived primary cultured chondrocytes or cultured chondrocytes or Runx2/Cba1- and p53-deficient mouse-derived chondrocyte cell lines that can be employed for the aforementioned technique for searching for a gene. The Runx2/Cbfa1-deficient mouse-derived primary cultured chondrocytes are obtained by treating the skeleton of a Runx2/Cbfa1-deficinet mouse at day 18.5 of embryonic development with trypsin and collagenase. The Runx2/Cba1- and p53-deficient mouse-derived chondrocyte cell lines were established by treating the skeleton of a Runx2/Cba1- and p53-deficient mouse at day 18.5 of embryonic development with trypsin and collagenase, and repeating cloning 3 or 4 times. Methods for establishing the aforementioned primary chondrocytes and chondrocyte cell lines are known, and such cells or cell lines can be obtained in accordance with other conventional techniques.

Preferable examples of Runx2/Cba1- and p53-deficient mouse-derived chondrocyte cell lines include, but are not limited to, RU-1 and RU-22. The RU-1 and RU-22 cell lines are deposited at the International Patent Organism Depositary of the National Institute of Advanced Industrial Science and Technology (Tsukuba Central 6, 1-1-1 Higashi, Tsukuba, Ibaraki, Japan) under the Budapest Treaty as of Aug. 5, 2003 (the original deposit), under the accession numbers FERM BP-10137 (the RU-1 cell line) and FERM BP-10138 (the RU-22 cell line).

[Polypeptide]

The polypeptide of the downstream gene of Runx2/Cbfa1 according to the present invention includes those represented by SEQ ID NO: 2, 4, 6, 8, 10, 12, 14, 16, 18, 20, 22, 24, 26, 28, 30, 32, 34, 36, 38, 40, 42, 44, 46, 48, 50, and 52. Polypeptide sequences can be found by searching public databases. The polypeptide sequences that are not registered with public databases can be easily deduced based on nucleotide sequences by open reading frame search. Further, a polypeptide shown in SEQ ID NO: 2, 4, 6, 8, 10, 12, 14, 16, 18, 20, 22, 24, 26, 28, 30, 32, 34, 36, 38, 40, 42, 44, 46, 48, 50, or 52 or a polypeptide registered with a public database may be employed if a new open reading frame has been discovered. Such polypeptide can be altered if functions (i.e., functions of stimulating or inhibiting cartilage differentiation) are not significantly changed. Examples of such alteration include substitution, deletion, or addition of one or several amino acid residues and modification of the amino or carboxyl group. Further, the polypeptide of the present invention also includes a polypeptide having an amino acid sequence having 65% or more homology to the amino acid sequence shown in SEQ ID NO: 2, 4, 6, 8, 10, 12, 14, 16, 18, 20, 22, 24, 26, 28, 30, 32, 34, 36, 38, 40, 42, 44, 46, 48, 50, or 52 and involving no significant changes in its functions (i.e., functions of stimulating or inhibiting cartilage differentiation).

Alternatively, a polypeptide encoded by HCK that had been demonstrated to be the downstream gene of Runx2/Cbfa1 in the present invention can be obtained as a commercially available protein (polypeptide) (e.g., Cat. No. P2908, Invitrogen). Also, the polypeptide encoded by GALNT3 that had been demonstrated to be the downstream gene of Runx2/Cbfa1 in the present invention can be obtained via purification in accordance with a conventional technique (J Biol Chem., Sep. 19, 1997; 272 (38): 23503-14).

The polypeptide of the present invention itself can be used in a pharmaceutical composition for regulating its functions in organisms (for example, cartilage differentiation or osteoarthritis development). Also, the polypeptide of the present invention can be used for screening for a compound capable of regulating its functions, such as an inhibitor, antagonist, or activator, or for obtaining an antibody reacting therewith. Further, the polypeptide of the present invention can also be used as a reagent.

[Recombinant Vector]

A recombinant vector can be obtained by inserting a polynucleotide encoding the downstream gene of Runx2/Cbfa1 of the present invention into an appropriate vector DNA. Vector DNA may be obtained from nature, or a vector that partially lacks DNAs that are not required for proliferation may be employed. For example, a vector derived from ColE1 or a vector derived from a lambda phage may be used. The DNA of the present invention can be inserted into the vector DNA using a conventional technique. For example, DNA is cleaved at given sites with adequately selected restriction enzymes, the resultant is mixed with vector DNA that had been treated in the same manner, and they are then ligated using a ligase.

[Transformant]

The novel polypeptide according to the present invention and derivatives therefrom can be obtained via gene recombination techniques using a cell-free protein expression system or conventional host cells, such as E. coli, yeast, Bacillius subtilis, insect cells, or animal cells. Transformation can be carried out in accordance with conventional techniques. For example, a plasmid, chromosome, or virus is used as a replicon to transform a host cell. From the viewpoint of gene stability, integration of genes into chromosomes is preferable. Autonomous replication involving the use of an episome is simply employed. A vector is adequately selected in accordance with a type of a host cell, and a vector is constituted by a gene sequence of interest and a sequence carrying information concerning replication and regulation of the former gene sequence. Constituents are selected depending on the type of the host cell, i.e., a procaryotic or eucaryotic cell, and a promoter, a ribosome binding site, a terminator, a signal sequence, an enhancer, and the like are combined in accordance with conventional techniques.

A transformant can be used for preparing the polypeptide of the present invention by culturing of host cells under conventional conditions optimal therefor. Culture may be conducted while determining the biological activity of a novel polypeptide to be expressed and produced as an indicator. Alternatively, subculture or batch culture may be carried out by employing the amount of transformants in a medium as an indicator.

[Antibody]

An antibody against a protein encoded by the downstream gene of Runx2/Cbfa1 can be used for diagnosing and treating a bone and/or joint disease. For example, Western blotting, the immunoprecipitation method, or ELISA involving the use of an antibody that binds to the downstream gene of Runx2/Cbfa1 can be adopted for diagnosis. An antibody that controls activity of the protein encoded by the gene of interest can be used for treatment.

The antibody used for the aforementioned purpose can be obtained in accordance with conventional techniques. The antibody used in the present invention can be a polyclonal or monoclonal antibody (Milstein C. et al., Nature, 1983, 305, 537-540). For example, a polyclonal antibody against the downstream gene of Runx2/Cbfa1 can be obtained by collecting serum from a mammalian animal that had been sensitized using the antigen polypeptide or peptide encoded by the downstream gene. Also, an immunocyte may be collected from the mammalian animal that had been sensitized using the antigen polypeptide or peptide encoded by the downstream gene, the collected cell is fused with a myeloma cell to prepare a hybridoma, the resulting hybridoma is subjected to cloning, and an antibody is recovered from the culture product to prepare a monoclonal antibody.

Specific examples include antibodies against proteins encoded by HCK or Tem8, which had been demonstrated to be the downstream genes of Runx2/Cbfa1 in the present invention. These antibodies, such as an anti-HCK antibody (Cat. No. 610277, BD Biosciences) and an anti-Tem8 antibody (Cat. No. 200C1339, Abcam) are commercially available. Antibodies disclosed in known literature (Mol Cell Biol., January 1997, 17 (1): 230-9) or antibodies prepared in the manner described in such literature can also be used as anti-Nopp140 antibodies or the like.

Proteins encoded by the downstream genes of Runx2/Cbfa1 may be detected using adequately labeled antibodies. Alternatively, proteins can be indirectly detected with the use of labeled substances that specifically bind to such antibodies, such as Protein A or Protein G. instead of labeling antibodies. A specific example of a detection method is ELISA.

Antigen proteins or partial peptides thereof can be obtained by, for example, inserting the downstream gene of Runx2/Cbfa1 or part thereof into an expression vector, introducing the vector into an adequate host cell to prepare a transformant, culturing the transformant to allow the expression of a recombinant protein, and purifying the expressed recombinant protein from the culture product or culture supernatant. Alternatively, an oligopeptide consisting of the amino acid sequence encoded by the aforementioned gene or a partial amino acid sequence of the amino acid sequence encoded by full-length cDNA can be chemically synthesized and used as an immunogen. Examples of animals to be immunized include mice, rats, rabbits, goats, horses, and hamsters.

[Anti Sense]

If information concerning a full-length gene sequence is available, a person skilled in the art can design an antisense oligo (polynucleotide) that inhibits functions of the gene. Even if only partial gene sequence information is available, an antisense oligo can also be designed. Concerning mouse Noppl40, which had been found to play a stimulatory role in cartilage differentiation in the present invention, for example, an antisense oligo such as an antisense oligo candidate sequence 5′-GCG CAA GCC GGT ATC CGC CAT-3′ (SEQ ID NO: 112) can be designed based on the sequence 5′-CGG AGC ATG GCG GAT ACC GGC TTG CGC CGC GTG-3′ (SEQ ID NO: 111) located in the vicinity of the initiation codon of Nopp140. An antisense oligo undergoes various forms of modification or binding in order to avoid decomposition in the cells. A person skilled in the art can select a suitable antisense oligo form. Examples of such forms include naturally occurring (D-oligo), phosphorothioate (S-oligo), methylphosphonate (M-oligo), phosphoroamidate (A-oligo), 2′-O-methyl (D-oligo), morpholidate (Mo-oligo), and polyamide nucleic acid forms. The antisense oligo sequence has 10 to 70 nucleotides in length, and preferably 15 to 30 nucleotides in length. The antisense oligo thus prepared can, for example, inhibit the functions of Nopp140, and it can be used as a therapeutic agent for a bone and/or joint disease (preferably osteoarthritis).

[RNAi]

RNA interference (RNAi) refers to a phenomenon of strongly inhibiting the expression of a target gene by degrading RNA of the target gene using double-stranded RNA molecules with 21 to 23 bps containing the sequence identical to the RNA of the target. Accordingly, the double-stranded RNA molecule that has the same nucleotide sequence as mRNA of the downstream gene of Runx2/Cbfa1 can be employed to inhibit the expression of the downstream gene of Runx2/Cbfa1. In order to achieve the RNAi effects, it is preferable to use double-stranded RNA molecules having nucleotide sequences comprising at least 20 continuous nucleotides. The double-strand structure may be composed of 2 different strands or the double-strand structure can be formed by one strand via a RNA stem-loop. In the present specification, the double-stranded RNA capable of such RNAi phenomenon can be employed as RNAi molecules.

In the case of mouse Noppl40, for example, 2 RNA strands; i.e., 5′-AUG GCG GAU ACC GGC UUG CGC-3′ (SEQ ID NO: 113) and a complementary strand thereof 5′-GCG CAA GCC GGU AUC CGC CAU-3′ (SEQ ID NO: 114), are synthesized from the sequence located in the vicinity of the initiation codon, and they are annealed to each other. Thus, double-stranded RNA molecules can be prepared, and they can be used as RNAI molecules. Provision of a 2-nucleotide overhang at each 3′ end of such strands can enhance the inhibitory effects on gene expression (WO 01/75164).

Another example of such RNAI molecule that can be used is a commercially available RNAi molecule, such as an RNAi (siRNA) molecule that inhibits the expression of HCK (Cat. No. G-003 100-TK-02, Dharmacon Research Inc.).

A person skilled in the art may make various forms of modification concerning the sequence used for designing RNAi molecules, and the length and the structure thereof in order to design RNAi molecules having optimal inhibitory effects on gene expression. The RNAi molecules thus obtained can be used as therapeutic agents for a bone and/or joint disease (preferably osteoarthritis) by inhibition of the expression of the downstream gene of Runx2/Cbfa1.

[Activity Assay]

According to the present invention, screening of compounds, antibodies, and the like, and diagnosis can be carried out with the use of the biological activity of a protein encoded by the downstream gene of Runx2/Cbfa1 in a biological sample as an indicator as well as the expression level of the downstream gene of Runx2/Cbfa1. For example, an increase or decrease and change in activity levels can be employed for the diagnosis of diseases associated with the Runx2/Cbfa1 gene. Also, a low-molecular-weight compound, antibody, or the like that inhibits or activates biological activity may be screened for to obtain a compound or antibody that controls activity of the protein encoded by the downstream gene of Runx2/Cbfa1. The resultant can be used for treatment of a bone and/or joint disease (preferably osteoarthritis). Hereafter, general techniques for assaying activities of proteins are described.

(1) Transcription Factor or Transcription Regulatory Factor

A transcription factor is incubated at room temperature with double-stranded oligo DNA labeled with ³²P or the like that contains the target sequence of the transcription factor to bind them together. The sample after incubation is subjected to electrophoresis on SDS-free undenatured polyacrylamide gel, and the rate of migration of the labeled oligo DNA is evaluated by employing the radioactivity of ³²P or the like as an indicator. If the transcription factor exhibits binding activity to oligo DNA, the rate of migration of the labeled oligo DNA slows down, and the oligo DNA is shifted to the higher-molecular-weight. Also, an expression vector that comprises a reporter gene such as chloramphenicol acetyltransferase (CAT) ligated to the downstream of the target sequence and an expression vector that comprises a transcription factor gene ligated to the downstream of the response gene promoter of human cytomegalovirus (CMV) can be cotransfected into cell lines such as Hela or HEK293, a homogenate solution is prepared 48 hours thereafter, and the expression level of CAT is examined. Thus, the activity of the transcription factor can also be evaluated (Zhao F. et al., J. Biol. Chem. 276, 40755-40760, 2001).

Specifically, the polypeptide encoded by Nopp140 that had been demonstrated to be the downstream gene of Runx2/Cbfa1 in the present invention is known as a transcription factor that induces the expression of the alpha-1 acid glycoprotein (AGP) gene. Accordingly, conventional techniques such as reporter gene assay that employs the AGP promoter can also be employed (Mol Cell Biol., January 1997, 17 (1): 230-9; and J Biol Chem., October 2002, 18, 277 (42): 39102-11).

(2) Kinase

Kinase is added to a buffer containing the myelin basic protein as a substrate (20 mM HEPES, pH 7.5, 10 mM MgCl₂, 2 mM dithiothreitol, and 25 μM ATP), [γ-³²P]ATP is added thereto, and incubated at 37° C. for 10 minutes. The reaction is terminated with the use of Laemmli buffer 10 minutes later, the reaction solution is subjected to SDS polyacrylamide gel electrophoresis, the gel is dehydrated after the electrophoresis, and the radioactivity of the phosphorylated myelin basic protein is detected using an X-ray film (Park S. Y. et al., J. Biol. Chem. 275, 19768-19777, 2000).

Specifically, the polypeptide encoded by HCK that had been demonstrated to be the downstream gene of Runx2/Cbfa1 in the present invention is known to function as kinase, and activity thereof can be assayed in accordance with conventional techniques (J. Biol. Chem., Sep. 10, 1999, Vol. 274 (37), p. 26579).

(3) Secretory Factor

The cells that are to be subjected to activity assay and are assumed to contain receptors of secretory factors are stimulated with the secretory factors, and changes in the cells resulting therefrom are assayed. Methods for assaying changes in the cells are described below.

Cells are suspended in the Hank's balanced salt solution containing the calcium-sensitive fluorescent dye fura-2 so as to stimulate the cells with secretory factors. A stimulus-induced increase in the calcium level in the cells is assayed using a fluorescence detector such as the LS50B (Perkin Elmer) (Zhou N. et al., J. Biol. Chem., 2001, 276, 42826-42833).

The cells are stimulated with secretory factors, and cell proliferation resulting therefrom is evaluated based on thymidine uptake.

Activation of a transcription factor that is considered to transmit stimuli of the secretory factor can be evaluated based on the expression of the reporter genes such as luciferases (Piek E. et al., J. Biol. Chem., 2001, 276, 19945-19953).

(4) Receptor or Membrane Protein

Receptors or membrane proteins are subjected to forced expression in the cells, they are stimulated with samples that are assumed to comprise ligands (a culture solution of cells, blood serum, or the like), and changes resulting therefrom in cells are assayed in accordance with the method described in the “secretory factor” section.

Activity of a receptor can also be assayed by assaying the binding between the receptor and a substance (i.e., a ligand) that binds thereto and exerts its functions. A specific example of such receptor is a polypeptide encoded by Tem8, which had been demonstrated to be the downstream gene of Runx2/Cbfa1 in the present invention. Tem8 is also known as a receptor that binds to anthrax (J. Biol. Chem., Feb. 14, 2003, Vol. 278 (7), p. 5227), and the binding to the type VI collagen α-3 subunit is also known (Cancer Research, Feb. 1, 2004, Vol. 64 (3), p. 817). Accordingly, binding between these substances and Tem8 is assayed, and the assay result can be employed as an indicator for activity assay.

(5) Phosphatase-Regulating Factor

Phosphatase that is to be regulated is subjected to activity assay, and the activity of the protein for regulating phosphatase is assayed. Phosphatase activity can be assayed in the following manner. Phosphatase is added to a buffer containing p-nitrophenyl phosphate (pNPP) as a substrate (25 mM MES, pH 5.5, 1.6 mM dithiothreitol, and 10 mM pNPP), and the resultant is incubated at 37° C. for 30 minutes. Thirty minutes later, IN NaOH is added to terminate the reaction, and the absorbance at 405 nm resulting from pNPP hydrolysis is assayed (Aoyama K. et al., J. Biol. Chem., 2001, 276, 27575-27583).

(6) Enzymes (Other Than Kinase or Phosphatase)

A wide variety of enzymes exist in addition to the aforementioned kinases and phosphatases, and they are involved in maintenance or change in the functions or structures of organisms. Since an enzyme has specificity (i.e., substrate specificity, reaction specificity, or the like) for the substances that react therewith (e.g., a substrate, a reaction product, or the like) and the reaction mechanisms, a variety of methods for assaying activity have been formulated in accordance with enzyme types. Also, some enzymes may form enzyme groups (enzyme families) having similar structures, substrate specificity, or reaction specificity, and the members of an enzyme family occasionally share reactivity with a substrate, an inhibitor, or an activator. Thus, a novel system for evaluating enzyme activity may be developed with the use of the method for assaying activities of enzymes that belong to the same enzyme family. When the downstream gene of Runx2/Cbfa1 obtained by the method of the present invention is an enzyme, accordingly, a method for assaying its activity can be developed with the use of conventional techniques or the methods for assaying enzymes of the same enzyme family.

A specific example thereof is a case of GALNT3, which was demonstrated to be the downstream gene of Runx2/Cbfa1 in the present invention. GALNT3 (ppGaNTase-T3) is a glycosyltransferase that belongs to the ppGaNTase family, and the activity thereof can be assayed by determining the reaction that occurs when adding GaINAc to a peptide such as MUC-2 or HIV-H3 using a UDP-GalNAc substrate (J. Biol. Chem., Jul. 19, 1996, Vol. 271 (29), p. 17006). Also, the GALNT3 protein can be purified in accordance with conventional techniques (J Biol Chem., Sep. 19, 1997, 272 (38): 23503-14). At present, 24 enzyme species of the ppGaNTase enzyme family has been reported (Glycobiology, January 2003, 13 (1): IR-16R). Since the ppGaNTase family including GALNT3 is reported to be capable of regulating enzyme activity of low-molecular-weight compounds, an activity inhibitor or activator may be obtained based on such information.

[Diagnostic Method]

In the diagnostic method of the present invention, a biological sample obtained from a subject is generally employed as a test sample. A biological sample is preferably a blood sample. Whole blood, or blood plasma or serum obtained therefrom can be used as a blood sample. In the present invention, synovial fluid, a piece of joint cartilage, synovial tissue, or the like obtained via biopsy can be used as a biological sample in addition to blood. Methods for obtaining such biological samples are known.

When the biological sample is a cell, such as a piece of joint cartilage or synovial tissue, a lysate can be prepared and used as a sample for immunological assay for the aforementioned proteins. Alternatively, mRNA may be extracted from this lysate and used as a sample for assaying mRNA for the aforementioned gene. Commercially available kits are conveniently used for extracting a lysate or mRNA of a biological sample. A liquid biological sample such as blood or synovial fluid may be diluted with a buffer or the like, if necessary, and the resultant can be used as a sample for assaying for proteins or genes.

A lysate prepared from the aforementioned biological sample can be used as a sample for immunological assay of the protein encoded by the downstream gene of Runx2/Cbfa1. mRNA extracted from such lysate can be used as a sample for assaying mRNA for the downstream gene of Runx2/Cbfa1. Commercially available kits are conveniently used for extracting a lysate or mRNA of a biological sample. When the protein encoded by the downstream gene of Runx2/Cbfa1 is secreted in the blood or synovial fluid, the amounts of target proteins in a body fluid sample such as blood or serum of the subject may be assayed to compare the expression levels of the genes encoding the same. If necessary, the aforementioned sample can be diluted with a buffer or the like and used in the method according to the present invention.

When mRNA is assayed, the measured expression level of the downstream gene of Runx2/Cbfa1 in the present invention can be corrected in accordance with conventional techniques. This correction enables the comparison of variations in gene expression levels in the cells. The measured expression levels of the downstream genes of Runx2/Cbfa1 in the present invention in the cells are corrected based on the measured expression levels of genes the expression levels of which do not significantly vary in cells in the biological samples (e.g., a housekeeping gene). Examples of genes the expression levels of which do not significantly vary include β-actin and GAPDH.

Further, the present invention provides a reagent (a diagnostic composition) used for the diagnostic method of the present invention. Specifically, the present invention relates to a diagnostic reagent for a bone and/or joint disease comprising a polynucleotide containing the nucleotide sequence of the downstream gene of Runx2/Cbfa1, an oligonucleotide having a nucleotide sequence complementary to the complementary strand of the polynucleotide and composed of at least 15 nucleotides, a DNA vector containing such polynucleotide, or a transformant transformed with such DNA vector. Also, the present invention relates to a diagnostic reagent for a bone and/or joint disease comprising a protein (polypeptide) encoded by the downstream gene of Runx2/Cbfa1, an antibody that recognizes a peptide containing the amino acid sequence of the protein, an antisense polynucleotide, or an RNAi molecule.

The oligonucleotide or antibody that constitutes the reagent of the present invention may be adequately labeled in accordance with an assay format. Also, the oligonucleotide or antibody that constitutes the reagent of the present invention may be immobilized on an adequate support in accordance with an assay format. The reagent of the present invention may be combined with additional elements required for detection or storage in addition to the aforementioned oligonucleotide or antibody and prepared in the form of a diagnostic kit. Additional elements (1) to (6) that can constitute the kit are listed below. These elements can be previously mixed according to need. If necessary, a preservative or antiseptic agent can be added to each element.

(1) A buffer for diluting the reagent or biological sample

(2) A positive control

(3) A negative control

(4) A substrate for assaying the labeling

(5) A reaction vessel

(6) The instructions describing the assay protocol

In the present invention, diagnosis of a bone and/or joint disease includes the following diagnosis, for example. Even if a patient exhibits symptoms suspected to indicate a bone and/or joint disease (preferably osteoarthritis) but cannot be diagnosed as having osteoarthritis by a general diagnostic procedure, by the diagnostic procedure according to the present invention, whether or not the patient is afflicted with osteoarthritis can be easily determined. More specifically, when a patient suspected of osteoarthritis exhibits an increase or decrease in the expression level of the downstream gene of Runx2/Cbfa1, the causal factor of such symptom is highly likely to be osteoarthritis.

Also, the present invention enables diagnosis whether or not the bone and/or joint disease (preferably osteoarthritis) is ameliorated. Specifically, the diagnostic method according to the present invention is useful for determining the therapeutic effects on osteoarthritis. More specifically, an increase or decrease in the expression level of the downstream gene of Runx2/Cbfa1 in a patient suspected of osteoarthritis indicates a possibility of the further progression or amelioration of osteoarthritis.

Further, the severity of osteoarthritis can be determined based on differences in the expression levels. Specifically, the degree of increase in the expression level of the downstream gene of Runx2/Cbfa1 may be correlated with the degree of severity of osteoarthritis.

[Transgenic Animal and Animal Model of Bone and/or Joint Disease]

The present invention relates to an animal model of a bone and/or joint disease, and preferably an animal model of osteoarthritis, including a transgenic nonhuman animal in which the expression level of the downstream gene of Runx2/Cbfa1 or a gene functionally equivalent thereto is enhanced systemically, or preferably cartilage-specifically. According to the present invention, the downstream gene of Runx2/Cbfa1 was found to stimulate or inhibit cartilage differentiation. Accordingly, an animal in which the expression level of the downstream genes of Runx2/Cbfa1 stimulating cartilage differentiation is artificially enhanced also exhibits stimulated cartilage differentiation, which is observed in the synovial cartilage of osteoarthritis. Thus, such animal can be employed as an animal model of osteoarthritis.

The present invention relates to an animal model of a bone and/or joint disease, and preferably an animal model of osteoarthritis or chronic rheumatism, including a transgenic nonhuman animal in which the expression level of the downstream gene of Runx2/Cbfa1 or a gene functionally equivalent thereto is lowered systemically, or preferably cartilage-specifically. The animal in which the expression levels of the downstream genes of Runx2/Cbfa1 inhibiting cartilage differentiation are artificially lowered also exhibits stimulated cartilage differentiation, which is observed in the osteoarthritic joint cartilage. Thus, such animal can be employed as an animal model of osteoarthritis.

In the present invention, functionally equivalent genes refer to those encode proteins having activities equivalent to those of proteins encoded by the downstream genes of Runx2/Cbfa1, which have been revealed. A representative example of the functionally equivalent gene of a test animal is a counterpart of the downstream gene of Runx2/Cbfa1 intrinsic to the test animal species.

Further, the present invention relates to a method for preparing an animal model of a bone and/or joint disease (preferably osteoarthritis) by the administration of the protein encoded by the downstream gene of Runx2/Cbfa1 or an antibody against the protein.

At the outset, the downstream gene of Runx2/Cbfa1 can induce stimulated cartilage differentiation by an increase or a decrease in its level of expression, and it can further preferably induce osteoarthritis. The correlation between the gene expression level and its influence on cartilage differentiation can be evaluated by examining whether or not the protein encoded by the gene of interest stimulates or inhibits cartilage differentiation. More specifically, an increased expression level of the downstream genes of Runx2/Cbfa1 stimulating cartilage differentiation (e.g., SEQ ID NO: 5 or 1) may result in stimulated cartilage differentiation and induction of osteoarthritis. In contrast, a decreased expression level of the downstream genes of Runx2/Cbfa1 inhibiting cartilage differentiation (e.g., SEQ ID NO: 3, 15, or 25) may result in stimulated cartilage differentiation and induction of arthritis.

In addition to the transgenic animal, the aforementioned increased gene expression level can be achieved by the administration of the protein encoded by such gene to an animal. This may induce osteoarthritis, and such animal may. be used as an animal model of osteoarthritis. The protein encoded by the aforementioned gene may be a full-length protein or a protein consisting of a partial sequence containing an active site, as long as it has equivalent functions.

In addition to the transgenic animal, the aforementioned decreased gene expression level can be achieved by the administration of a substance that inhibits activity of the protein encoded by the gene of interest or a substance that decreases the expression level of the gene of interest to an animal. This may induce osteoarthritis, and such animal may be used as an animal model of osteoarthritis. Specific examples of substances that inhibit activity of the protein encoded by the gene of interest include activity inhibitors such as antibodies or compounds. Also, a decoy partial polypeptide or the like that consists of a region binding to a ligand and cannot transduce signals into the cells (i.e., an extracellular domain soluble receptor) can be used. Examples of substances decreasing the expression level of the gene include an antisense nucleic acid, a ribozyme, and an RNAI molecule. In the case of a transcription factor, a decoy nucleic acid can be designed based on a DNA sequence specific for the promoter recognized by such transcription factor. The thus designed decoy is considered to be capable of inhibiting the activation of a transcription factor, and it can be used for preparing the animal model of osteoarthritis or a pharmaceutical preparation for osteoarthritis.

The animal model of osteoarthritis according to the present invention is useful for elucidating changes in an individual suffered from osteoarthritis in vivo. Further elucidation of functions of the downstream gene of Runx2/Cbfa1 and evaluation of a medicine targeting the gene of interest with the use of the animal model of osteoarthritis is significant.

The animal model of osteoarthritis according to the present invention is useful for elucidation of the mechanisms of osteoarthritis and for the test of safety of the screened compound. If the animal model of osteoarthritis according to the present invention exhibits cartilage degeneration and exhibits changes in the measured value of some osteoarthritis-associated marker or cartilage differentiation marker, for example, a system for screening for a compound that can recover such change can be constructed.

In the present invention, the term “increased expression level” refers to any of the following conditions: where the downstream gene of Runx2/Cbfa1 is introduced as a extrinsic gene and forcedly expressed; where transcription of the downstream gene of Runx2/Cbfa1 intrinsic to the test animal and translation thereof into a protein are enhanced; and where degradation of the protein as a translation product is suppressed.

In the present invention, the term “decreased expression level” refers to any of the following conditions: where transcription of the downstream gene of Runx2/Cbfa1 intrinsic to the test animal and translation thereof into a protein are blocked; or where degradation of the protein as a translation product is enhanced. The gene expression level can be determined via, for example, DNA chip analysis based on differences in signal intensity or real-time PCR analysis. Activity of the protein as a translation product can be determined in comparison with activity under normal conditions.

Representative examples of transgenic animals include: animals into which the target gene has been introduced and forcedly expressed; animals in which the downstream genes of Runx2/Cbfa1 have been knocked out; and animals in which the downstream genes of Runx2/Cbfa1 have been substituted (knocked in) with other genes. In the present invention, transgenic animals into which an antisense nucleic acid to the downstream gene of Runx2/Cbfa1, a ribozyme, DNA encoding a polynucleotide exhibiting the RNAi effects, and DNA functioning as a decoy nucleic acid have been introduced can also be used as the transgenic animals. Examples of other transgenic animals include animals prepared by introducing mutation into the coding region of the downstream gene of Runx2/Cbfa1 to enhance or inhibit its activity or to modify the amino acid sequence to make it likely or unlikely to degrade. Mutation in the amino acid sequence may include substitution, deletion, insertion, or addition. Also, the transcription regulatory region of the gene may be mutated to regulate the expression of the downstream gene of Runx2/Cbfa1 according to the present invention.

A method for obtaining a transgenic animal by targeting a given gene is a known technique. Specific examples of methods for obtaining a transgenic animal include: a method wherein mixture of a gene and an egg is treated with calcium phosphate; a method wherein a gene is directly introduced into the nucleus of the pronuclear stage egg using a micropipette under a phase contrast microscope (microinjection, U.S. Pat. No. 4,873,191); and a method involving the use of an embryonic stem (ES) cell. Also, a method wherein a gene is inserted into a retrovirus vector to infect the egg, a method wherein the gene is introduced into the egg via sperm (i.e., the sperm vector method), and other methods have been developed. The sperm vector method is a technique of gene recombination wherein extrinsic genes are made to adhere to sperm or incorporated into sperm cells via electroporation or other means, and the sperms are then allowed to fertilize eggs, thereby introducing the extrinsic genes (Lavitranoet M. et al., Cell, 1989, 57, 717-723).

When a promoter the transcription of which is regulated by an adequate substance such as an agent is used as an expression vector, the expression level of the extrinsic downstream genes of Runx2/Cbfa1 in the transgenic animal can be regulated by the administration of the aforementioned substance. An example of such promoter is, but is not limited to, a type II collagen promoter.

Transgenic animals that are used as the animal models of osteoarthritis in the present invention can be produced from all types of vertebrates except for humans. Specifically, transgenic animals are produced from vertebrates, such as mice, rats, rabbits, miniature pigs, goats, sheep, monkeys, dogs, cats, or cattle by various genes or by alteration in expression levels of various genes.

[Method for Screening for Candidate Compounds of Therapeutic Agents or Prophylactic Agents for a Bone and/or Joint Disease (In vivo)]

The present invention relates to a method for screening for candidate compounds for therapeutic agents and/or prophylactic agents for a bone and/or joint disease (preferably osteoarthritis). In the present invention, the downstream gene of Runx2/Cbfa1 was found to stimulate or inhibit cartilage differentiation. Osteoarthritis involves the stimulated differentiation of permanent joint cartilage. Concerning the downstream genes of Runx2/Cbfa1 capable of inhibiting cartilage differentiation (e.g., a gene having the nucleotide sequence shown in SEQ ID NO: 5 or 9), accordingly, screening for a compound that lowers the expression level of such gene enables the provision of therapeutic agents and/or prophylactic agents for osteoarthritis. Concerning the downstream genes of Runx2/Cbfa1 capable of inhibiting cartilage differentiation (e.g., a gene having the nucleotide sequence shown in SEQ ID NO: 3, 15, or 25), accordingly, screening for a compound that enhances the expression level of such gene enables the provision of therapeutic agents and/or prophylactic agents for osteoarthritis.

In the present invention, a compound that enhances or lowers the gene expression level refers to a compound that enhances or inhibits gene transcription, translation, or expression of protein activity. A compound that lowers the gene expression level in the present invention is a compound having functions that inhibit any of such processes.

The method for screening for candidate compounds for therapeutic agents and/or prophylactic agents for a bone and/or joint disease (preferably osteoarthritis) according to the present invention may be carried out in vivo or in vitro. Such screening can be carried out in accordance with, for example, the following procedure:

(1) a step of administrating the candidate compound to the test animal;

(2) a step of assaying the expression level of the downstream gene of Runx2/Cbfa1 in the biological sample obtained from the test animal; and

(3) a step of selecting a compound that lowers the expression level of the downstream genes of Runx2/Cbfa1 in case that the downstream genes stimulate cartilage differentiation or a compound that enhances the expression level of the downstream gene of Runx2/Cbfa1 in case that the downstream genes inhibit cartilage differentiation, when compared with a control to which no candidate compound has been administered.

In the screening method according to the present invention, the downstream genes of Runx2/Cbfa1 or genes functionally equivalent thereto can be employed. In the present invention, functionally equivalent genes encode proteins having activities equivalent to those of proteins encoded by the downstream genes of Runx2/Cbfa1, which have been revealed. A representative example of such a functionally equivalent gene of a test animal is a counterpart of the downstream gene of Runx2/Cbfa1 intrinsic to the test animal species.

Test animals used in the screening method according to the present invention includes transgenic animals of a bone and/or joint disease (preferably osteoarthritis) according to the present invention, described in the section [Transgenic animal and animal model of bone and/or joint disease] and animal models of a bone and/or joint disease (preferably osteoarthritis) prepared by the administration of a polypeptide encoded by the downstream gene of Runx2/Cbfa1 or an antibody against the aforementioned polypeptide. Other conventional animal models of osteoarthritis can also be used. Examples of conventional animal models of osteoarthritis include models of spontaneous osteoarthritis (e.g., the STR/ORT mouse) and models with excised anterior cruciate ligament (e.g., mice, rats, rabbits, or dogs).

A person skilled in the art can identify the homologue in an animal species other than mice, based on the disclosure of the present invention. For example, genes (or proteins) of other species that are highly homologous to the nucleotide sequence or amino acid sequence of the human homologue can be discovered by homology search. Alternatively, a homologue in other animal species can be isolated by hybridization with the downstream gene of Runx2/Cbfa1. In the screening method that involves the use of animal models to which human genes have been introduced, human genes may be occasionally detected as the downstream genes of Runx2/Cbfa1, in addition to the homologues of the animal in question.

By the administration of a drug candidate compound to the test animal and the monitoring of the effects of the compound on the expression of the downstream gene of Runx2/Cbfa1 in the biological sample derived from the test animal, the effects of the drug candidate compound on the expression level of the downstream gene of Runx2/Cbfa1 can be evaluated. Change in the expression level of the downstream gene of Runx2/Cbfa1 in the biological sample obtained from the test animal can be monitored in the same manner as that of the diagnostic method according to the present invention. Based on the evaluation results, a drug candidate compound that lowers the expression level of the downstream genes of Runx2/Cbfa1 which stimulate cartilage differentiation or a drug candidate compound that enhances the expression level of the downstream gene which inhibit cartilage differentiation may be selected to screen for the drug candidate compound.

Through such screening procedure, drugs that are involved in the expression of the downstream gene of Runx2/Cbfa1 in diverse ways can be selected. Specifically, a drug candidate compound having functions as described below can be discovered. Concerning the downstream genes of Runx2/Cbfa1 which stimulate cartilage differentiation:

(1) inhibition of the signaling pathway that induces the expression of the downstream genes of Runx2/Cbfa1;

(2) inhibition of transcription activity of the downstream genes of Runx2/Cbfa 1; and

(3) inhibition of stabilization or acceleration of degradation of the transcription product of the downstream genes of Runx2/Cbfa1.

Concerning the downstream genes of Runx2/Cbfa1 which inhibit cartilage differentiation:

(1) activation of the signaling pathway that induces the expression of the downstream genes of Runx2/Cbfa1;

(2) acceleration of transcription activity of the downstream genes of Runx2/Cbfa1; and

(3) stabilization or inhibition of degradation of the transcription product of the downstream genes of Runx2/Cbfa1.

[Method for Screening for Candidate Compounds for Therapeutic Agents and/or Prophylactic Agents for a Bone and/or Joint Disease (In vitro)]

In in vitro screening, for example, the candidate compound is brought into contact with a cell that expresses the downstream gene of Runx2/Cbfa1, a compound that lowers the expression level of the downstream genes of Runx2/Cbfa1 in the case that the downstream genes stimulate cartilage differentiation or a compound that enhances the expression level of the downstream gene in case that the downstream genes inhibit cartilage differentiation are selected. Such screening can be carried out in accordance with, for example, the following steps:

(1) a step of bringing the candidate compound into contact with the cell that expresses the downstream gene of Runx2/Cbfa1;

(2) a step of assaying the expression level of the downstream gene of Runx2/Cbfa1; and

(3) a step of selecting a compound that lowers the expression level of the downstream genes of Runx2/Cbfa1 in the case that the downstream genes stimulate cartilage differentiation or a compound that enhances the expression level of the downstream gene of Runx2/Cbfa1 in the case that the downstream genes inhibit cartilage differentiation, when compared with a control, which has not been brought into contact with candidate compound.

In the present invention, the cell that expresses the downstream gene of Runx2/Cbfa1 can be obtained by inserting the downstream gene of Runx2/Cbfa1 into an adequate expression vector and introducing the vector into an adequate host cell. Any vectors and host cells may be used as long as the downstream gene of Runx2/Cbfa1 can be expressed in the host cells. Examples of host cells that can be used in a host-vector system include E. coli, yeast, insect cells, and animal cells. Suitable vectors that can be used in each host cell can be adequately selected.

A vector can be introduced into a host cell via biological, physical, chemical, or other means. Examples of biological means include a method that involves the use of a virus vector, a method that involves the use of a specific receptor, the cell fusion method (hemagglutinating virus of Japan (HVJ)), a method using the polyethylene glycol (PEG), electrical cell fusion, and micronuclear fusion (chromosome transfer). Examples of physical means include microinjection, electroporation, and a method involving the use of a gene particle gun. Examples of chemical means include calcium phosphate precipitation, the liposome method, the DEAE dextran method, the protoplast method, erythrocyte ghost-mediated fusion, erythrocyte membrane ghost-mediated fusion, and microencapsulation.

In the screening method according to the present invention, a cell of the murine chondrocyte cell line, ATDC5, or the like can be used for a cell that expresses the downstream gene of Runx2/Cbfa1. The Cbfa1−/− and p53−/− mouse-derived chondrocyte cell lines that were established in the present invention can be used for host cells that express the downstream gene of Runx2/Cbfa1. Also, primary cultured chondrocytes derived from rats, rabbits, chickens, or mice can be used. The primary cultured chondrocytes can be obtained from joint cartilage and epiphyseal plates in accordance with conventional techniques.

According to the screening method of the present invention, the expression levels of the downstream gene of Runx2/Cbfa1 can be compared by detecting the mRNA levels of the proteins encoded by the genes as well as the expression levels of such proteins. In order to compare the expression levels based on the mRNA levels, the process of preparing mRNA samples mentioned above is carried out instead of the process of preparing protein samples. mRNA or proteins can be detected in accordance with conventional techniques as described above.

Further, a transcription regulatory region of the downstream gene of Runx2/Cbfa1 according to the present invention is obtained based on the disclosure of the present invention, and a reporter-assay system can be constructed. The term “reporter-assay system” refers to an assay system wherein the expression level of the reporter genes located downstream of the transcription regulatory region is employed as an indicator to screen for the transcription regulatory factor that functions in the transcription regulatory region.

Specifically, the present invention relates to a method for screening for a therapeutic agent and/or prophylactic agent for a bone and/or joint disease (preferably osteoarthritis) comprising the following steps (1) to (3), wherein the agent is targeted to the downstream gene of Runx2/Cbfa1 or a gene functionally equivalent thereto:

(1) a step of bringing a cell into contact with a candidate compound, wherein a vector containing the transcription regulatory region of the downstream gene of Runx2/Cbfa1 and a reporter gene expressed under the control of the transcription regulatory region has been introduced into the cell;

(2) a step of assaying activity of the reporter gene; and

(3) a step of selecting a compound that lowers the expression level of the reporter gene concerning the downstream genes of Runx2/Cbfa1 which stimulate cartilage differentiation or a compound that enhances the expression level of the reporter gene concerning the downstream genes of Runx2/Cbfa1 which inhibit cartilage differentiation when compared with a control, which has not been brought into contact with the candidate compound.

Examples of transcription regulatory regions include a promoter region, an enhancer region, and a CAAT box and a TATA box generally observed in promoter regions.

Examples of reporter genes that can be used include chloramphenicol acetyltransferase (CAT) genes, luciferase genes, and growth hormone genes.

Alternatively, the transcription regulatory regions of the downstream genes of Runx2/Cbfa1 according to the present invention can be obtained in the following manner. Specifically, human genomic DNA libraries such as a BAC library and a YAC library are first screened by the method involving PCR or hybridization based on the nucleotide sequence of cDNA disclosed in the present invention to obtain genomic DNA clones containing the sequences of the cDNA. Based on the obtained genomic DNA sequences, the transcription regulatory region of the cDNA disclosed in the present invention is deduced to obtain the transcription regulatory region. The obtained transcription regulatory region is cloned so as to be located upstream of the reporter gene to form a reporter construct. The resulting reporter construct is introduced into the cultured cell line to prepare a transformant for screening. The resulting transformant is brought into contact with the candidate compound, the transformant is compared with the control that has not been brought into contact with the candidate compound, and a compound that lowers the expression level of the reporter gene concerning the downstream genes of Runx2/Cbfa1 which stimulate cartilage differentiation or a compound that enhances the expression level of the reporter gene concerning the downstream genes which inhibit cartilage differentiation are selected. Thus, the screening method according to the present invention can be carried out.

The in vitro screening method of the present invention can be carried out via screening based on the activity of the downstream gene of Runx2/Cbfa1. Specifically, the present invention relates to a method for screening for a therapeutic agent and/or prophylactic agent for a bone and/or joint disease (preferably osteoarthritis) comprising the following steps, wherein the agent is targeted to the downstream gene of Runx2/Cbfa1 or a gene functionally equivalent thereto:

(1) a step of bringing a protein encoded by the downstream gene of Runx2/Cbfa1 into contact with a candidate compound;

(2) a step of assaying activity of the protein; and

(3) a step of selecting a compound that lowers the aforementioned activity concerning the downstream genes of Runx2/Cbfa1 which stimulate cartilage differentiation or a compound that enhances the aforementioned activity concerning the downstream genes of Runx2/Cbfa1 which inhibit cartilage differentiation when compared with a control, which has not been brought into contact with the candidate compound.

A compound having the activity of inhibiting the activity of the downstream genes of Runx2/Cbfa1 which stimulate cartilage differentiation can be screened for by employing the activity of the protein encoded by the downstream gene of Runx2/Cbfa1 in the present invention as an indicator. The thus obtained compound inhibits functions of such genes. As a result, stimulated cartilage differentiation observed in osteoarthritis can be inhibited to regulate the osteoarthritic symptoms. Activity of the protein encoded by the downstream gene of Runx2/Cbfa1 can be assayed in accordance with the aforementioned common techniques. A person skilled in the art can optimize the assay method by modifying the composition of the reagent, the composition of the buffer, the substrate, and the like.

A compound having activity of enhancing the activity of the downstream genes of Runx2/Cbfa1 which inhibit cartilage differentiation can be screened for. The thus obtained compound enhances functions of such genes. As a result, stimulated cartilage differentiation observed in osteoarthritis can be inhibited to regulate osteoarthritic symptoms.

Examples of candidate substances used for screening include compound samples synthesized via conventional chemical techniques, such as a steroid derivative, compound samples synthesized via combinatorial chemistry, mixtures comprising a plurality of compounds such as animal or plant tissue extracts or cultured microorganisms, and samples purified therefrom.

Polynucleotides, antibodies, cell lines, or animal models required for various screening procedures according to the present invention can be combined in advance to provide a kit. Such kit can be provided with a substrate compound used for label detection, a medium or container for cell culture, a positive or negative reference sample, and instructions of the use of the kit.

The compound selected by the screening method according to the present invention is useful as a therapeutic agent and/or prophylactic agent for a bone and/or joint disease (preferably osteoarthritis). Also, the antisense nucleic acid, a ribozyme, or the RNAi effects that can suppress the expression of the downstream genes of Runx2/Cbfa1 which stimulate cartilage differentiation may be useful for the therapeutic agent and/or prophylactic agent for osteoarthritis.

Further, antibodies that recognize peptides containing the amino acid sequences of proteins encoded by the downstream genes of Runx2/Cbfa1 which stimulate cartilage differentiation are useful for the therapeutic agents and/or prophylactic agents for osteoarthritis. Furthermore, proteins encoded by the downstream gene of Runx2/Cbfa1 which inhibit cartilage differentiation are also useful for the therapeutic agents and/or prophylactic agents for osteoarthritis.

[Pharmaceutical Preparations]

The therapeutic agent and/or prophylactic agent for a bone and/or joint disease (preferably osteoarthritis) according to the present invention comprises, as an active ingredient, the compound selected by the screening method. Such agent can be manufactured by mixture with a physiologically acceptable carrier, excipient, diluent, or the like. The therapeutic agent and/or prophylactic agent for osteoarthritis according to the present invention can be administered orally or parenterally to a patient aimed at ameliorating osteoarthritic symptoms.

Oral preparations can be the forms of, for example, granules, powders, tablets, capsules, solutions, emulsions, or suspensions. Parenteral injection may be, for example, subcutaneous, intramuscular, intra-articular cavity, or intraperitoneal injection.

When the active ingredient of the therapeutic agent and/or prophylactic agent to be administered is a protein, the gene encoding the same can be introduced into the organism by a technique of gene therapy to attain the therapeutic and/or prophylactic effects. It is a known technique to treat and/or prevent a disease by introducing the gene encoding the protein that yields the therapeutic and/or prophylactic effects into the organism and to allow the expression thereof.

Alternatively, an antisense nucleic acid, a ribozyme, or a polynucleotide that suppresses the expression of the aforementioned gene by the RNAi effects can be incorporated into a site downstream of an adequate promoter sequence, and the resultant can be administered as an expression vector for the antisense RNA, the ribozyme, or RNA that yields the RNAi effects. When such expression vector is introduced into the joint cartilage or synovial cells of the patient with osteoarthritis, the antisense nucleic acid to such gene, the ribozyme, or the polynucleotide that suppresses the expression of the aforementioned gene by the RNAi effects is expressed. This results in a lowered expression level of the gene, which in turn yields the therapeutic and/or prophylactic effects against osteoarthritis.

The antisense RNA refers to RNA having a nucleotide sequence complementary to the sense sequence of the gene. Suppression of the gene expression by antisense RNA necessitates the use of RNA having a nucleotide sequence consisting of 15 or more continuous nucleotides in general, such as 20 or more or 30 or more continuous nucleotides. For example, the antisense nucleic acid that can hybridize to a region containing an initiation codon is assumed to significantly suppress the expression of the gene of interest.

The ribozyme is RNA that has a catalytic action of cleaving RNA in a nucleotide-sequence-specific manner. The hammerhead or hairpin ribozyme is known. Such ribozyme is composed of a nucleotide sequence complementary to the region to be cleaved and a nucleotide sequence that maintains the structure necessary for exerting the catalytic activity. A nucleotide sequence complementary to the region to be cleaved can be an arbitrary sequence. By employing a nucleotide sequence complementary to the target gene in such region, accordingly, a ribozyme for regulating the expression of the downstream gene of Runx2/Cbfa1 can be designed.

The term “RNA interference (RNAI) effect” refers to the phenomenon whereby a double-stranded RNA molecule comprising a nucleotide sequence identical to that of mRNA forcedly suppresses the expression of such mRNA. Accordingly, the double-stranded RNAi molecule having a nucleotide sequence identical to that of mRNA of the downstream gene of Runx2/Cbfa1 can be used for suppressing the expression of the downstream gene of Runx2/Cbfa1. In order to attain the RNAi effects, it is preferable to use a double-stranded RNAi molecule comprising a nucleotide sequence consisting of at least 20 continuous nucleotides. The double strands may be composed of 2 different strands or the double-strand can be formed by one strand via a RNA stem-loop.

The complementary or identical nucleotide sequences among the antisense nucleic acid, the ribozyme, and the polynucleotide to realize the RNAi effects (i.e., the RNAi molecule) are not limited to completely complementary or identical nucleotide sequences. Such effects of suppressing the RNA expression are maintained if successfully high complementarity or identity is maintained. When a nucleotide sequence has identity of generally 70% or more, preferably 80% or more, more preferably 90% or more, and further preferably 95% or more (e.g., 98% or more identity) with reference to a given nucleotide sequence or a nucleotide sequence complementary thereto, their identity or complementarity can be successfully high.

The dosage varies depending on, for example, the age, sex, body weight, and symptoms of the patient, the therapeutic and/or prophylactic effects, the route of administration, the treatment period, or the type of active ingredient contained in the pharmaceutical composition. In general, dosage can be 0.1 mg to 500 mg, and preferably 0.5 mg to 20 mg, per adult. Since the dosage varies depending on various types of conditions, a dosage smaller than the lower limit of the aforementioned range may be occasionally sufficient, and a dosage exceeding the upper limit of the aforementioned range may be occasionally necessary.

### EXAMPLES

The present invention is hereafter described in detail with reference to examples, although the technical scope of the present invention is not limited thereto.

In the following examples, each procedure was carried out in accordance with the method described in the Molecular Cloning (Sambrook J, Fritsch E F and Maniatis T, Cold Spring Harbor Laboratory Press, 1989), unless otherwise specified. When commercialized reagents or kits were employed, they were used in accordance with the instructions thereof.

### Example 1

### Isolation of Primary Chondrocyte from Runx2/Cbfa1-Deficient Mouse Fetus and Establishment of Chondrocyte Cell Lines from Runx2/Cbfa1- and p53-Deficient Murine Embryonic Skeleton

(1) Sampling of Primary Chondrocyte Derived from Runx2/Cbfa1−/− Mouse

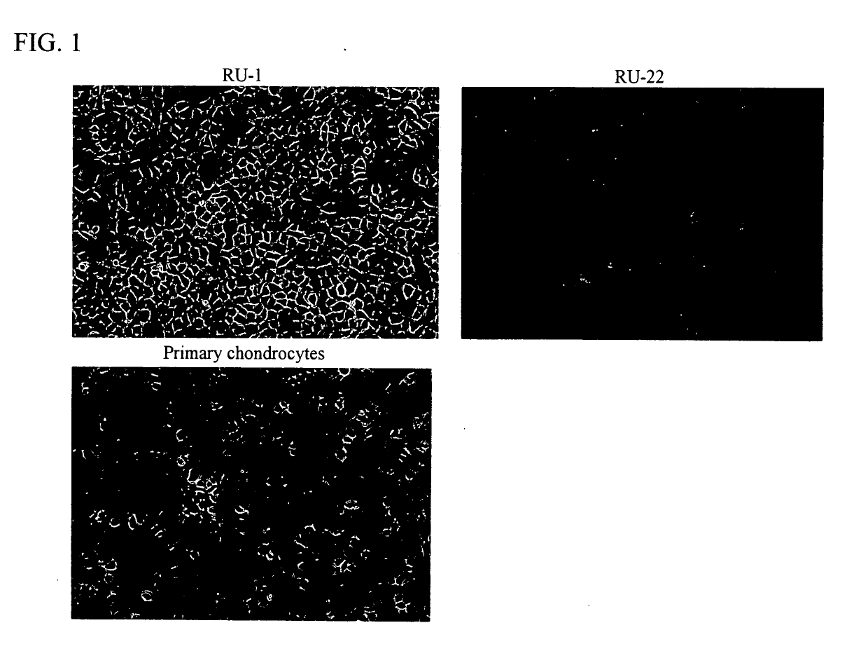

The Runx2/Cbfa1 knockout mice prepared by Komori et al. were used (Cell, 1997, 89, 755-764, JP Patent Publication (Unexamined) No. 10-309148 (1998)). The skeletons were collected from the Runx2/Cbfa1-homodeficient mice at day 18.5 of embryonic development, and the skeletons were treated with a solution containing 0.1% EDTA and 0.1% Trypsin (pH 7.4) at 37° C. for 60 minutes. Thereafter, the skeletons were treated with 1.5 mg/ml collagenase and alpha modified-minimum essential medium (αMEM) for 3.5 hours to obtain a cell suspension. The cell suspension was cultured on a collagen-coated dish containing Dulbecco's Modified Eagle's Medium/Ham's F12 (1:1) hybrid medium (Gibco BRL) containing 10% fetal bovine serum for 2 or 3 days for cell proliferation, and the resultant was used in the subsequent experiment. The morphology of the primary chondrocyte is shown in FIG. 1 (bottom row).

(2) Generation of Runx2/Cbfa1- and p53-Deficient Mouse

In order to establish chondrocyte cell lines that maintain the traits of undifferentiated chondrocytes, proliferative capacity, and viability, mice that were deficient in both the Runx2/Cbfa1 and p53 genes were generated to obtain chondrocytes. The p53-deficient mice generated by Gond Y. et al. were used (Biochem. Biophy. Res. Commun., 1994, 202, 830-837). Since the Runx2/Cbfa1-homodeficient mice (Runx2/Cbfa1−/−) die immediately after birth, they cannot be subjected to crossing. Accordingly, the Runx2/Cbfa1-heterodeficient mice (Runx2/Cbfa1±) were used for the generation of the Runx2/Cbfa1-deficient mice. The Runx2/Cbfa1-heterodeficient mice (Runx2/Cbfa1±) were subjected to crossing with the p53-deficient mice (p53−/−), and part of the tails of the resulting offspring mice was sampled to determine the genomic genes. Thus, the Runx2/Cbfa 1 -heterodeficient and p53-homodeficient mice (Runx2/Cbfa1± and p53−/−) were obtained. Thereafter, the Runx2/Cbfa1-heterodeficient and p53-homodeficient mice (Runx2/Cbfa1± and p53−/−) were subjected to crossing with each other, and the morphology of the resulting embryos and the genomic genes extracted from some tissues thereof were examined. Thus, Runx2/Cbfa1-homodeficient and p53-homodeficient mice (Runx2/Cbfa1−/− and p53−/−) were obtained.

(3) Preparation of Chondrocyte Cell Line Derived from Runx2/Cbfa1−/− and p53−/− Deficient Mouse

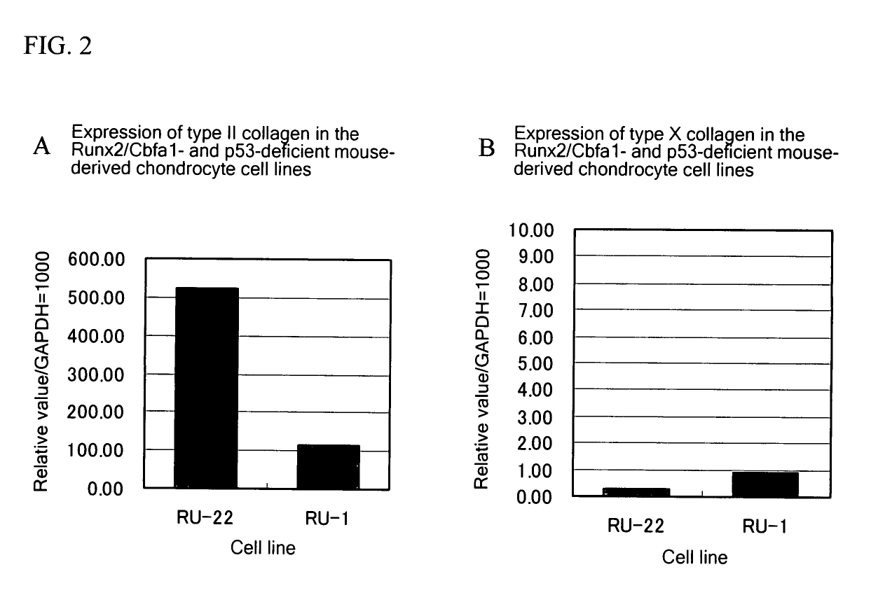

The skeletons were sampled from the Runx2/Cbfa1-homodeficient and p53-homodeficient mice at day 18.5 of embryonic development, and the sampled skeletons were treated with a solution containing 0.1% EDTA and 0.1% Trypsin (pH 7.4) at 37° C. for 60 minutes. Thereafter, the skeletons were treated with 1.5 mg/ml collagenase and alpha modified-minimum essential medium (αMEM) for 3.5 hours to obtain a cell suspension. The resulting cells were cultured on a dish containing 10% fetal bovine serum and Dulbecco's Modified Eagle's Medium (DMEM) at a cell density of 50 to 200 cells per 10-cm dish and allowed to form a colony, the resulting colonies were treated with trypsin/EDTA in a stainless steel cloning ring to pick up the colonies. Further, the resulting colonies were subjected to cloning 2 to 4 times via limiting dilution again, and cells that maintain proliferative capacity and viability were selected. The selected cells were examined for the expression of type II collagen expressed specifically in chondrocytes and the expression of type X collagen, the expression level of which is low in undifferentiated chondrocytes (by the method described in (4) below), and the candidates of the cell lines that maintain the traits of undifferentiated chondrocytes were selected. The selected candidate cell lines were subjected to 5 generations of subculture and then we confirmed that they did not exhibit changes in the traits of undifferentiated chondrocytes or lowering in proliferative capacity and viability. Thus, the obtained cell lines were verified to be capable of stably maintaining the properties thereof Finally, 2 types of Runx2/Cbfa1−/− and p53−/− mouse-derived chondrocyte cell lines, i.e., RU-1 and RU-22, were obtained through the aforementioned procedure. The morphologies of the obtained cell lines are shown in FIG. 1 (top row). (4) Expression analysis of type II collagen and type X collagen in RU-1 and in RU-22 In order to examine the differentiation stage and properties of the resulting cell lines, the expression patterns of type II collagen and type X collagen in RU-22 and in RU-1 were analyzed via real-time PCR. The RU-22 and RU-1 cell lines were cultured in 10% fetal bovine serum/Dulbecco's Modified Eagle's Medium (DMEM), and total RNA was purified using Isogen (Nippon Gene) when the cells became confluent. Total RNA was prepared in accordance with the method described in the instructions of Isogen. Further, single-stranded cDNA was synthesized from total RNA using a reverse transcriptase and an oligo (dT) primer. The resultant was used as a template to assay the expression level of type II collagen and type X collagen via real-time PCR analysis. Assay was conducted using the ABI PRISM 7700 (Applied Biosystems) in accordance with the instructions of the SYBR Green PCR Master Mix (Applied Biosystems). The sequences of the primers used are shown in Table 2. The Ct values obtained as a result of the assay were corrected with the expression of glyceraldehyde-3-phosphate dehydrogenase (GAPDH) and expressed as the relative value when GAPDH was determined to be 1,000 (FIG. 2).

TABLE 2
List of primers used for real-time PCR analysis (1)

| | | |
|---|---|---|
| | | SEQ |
| | | ID |
| Genes | Primer sequences | NO: |

| | | | |
|---|---|---|---|
| Runx2/Cbfal | Forward | CCGCACGACAACCGCACCAT | 53 |
| | Reverse | CGCTCCGGCCCACAAATCTC | 54 |
| Osteopontin (OSP) | Forward | CTCCAATCGTCCCTACAGTCG | 55 |
| | Reverse | CCAAGCTATCACCTCGGCC | 56 |
| Parathyroid hormone | Forward | TGTTTCTGCAATGGTGAGGTG | 57 |
| receptor (PTH/PTHrPR) | Reverse | GCGGCTCCAAGACTTCCTAAT | 58 |
| Osteocalcin (OC) | Forward | GCTGCCCTAAAGCCAAACTCT | 59 |
| | Reverse | AGAGGACAGGGAGGATCAAGTTC | 60 |
| Bone sialoprotein | Forward | TGGCGACACTTACCGAGCTT | 61 |
| (BSP) | Reverse | CCATGCCCCTTGTAGTAGCTGTA | 62 |
| Alkaline phosphatase | Forward | CTTGACTGTGGTTACTGCTGATCA | 63 |
| (ALP) | Reverse | GTATCCACCGAATGTGAAAACGT | 64 |
| Type II collagen | Forward | TGTCCCTCGGAAAAACTGGT | 65 |
| | Reverse | AGCCACCGTTCATGGTCTCT | 66 |
| Type X collagen | Forward | AGAACGGCACGCCTACGAT | 67 |
| | Reverse | AGGTAGCCTTTGCTGTACTCATCAT | 68 |
| Collagenase-3 | Forward | TCACCTGATTCTTGCGTGCT | 69 |
| (MMP-13) | Reverse | CTGTGGGTTATTATCAATCTTGTTTCTT | 70 |
| Interleukin-11 (IL-11) | Forward | GCATGTACAATGGCTGCGC | 71 |
| | Reverse | CAAGAGCTGTAAACGGCGG | 72 |
| Indian hedgehog (Ihh) | Forward | GAGACACCATTGAGACTTGACCAG | 73 |
| | Reverse | CACCAAGATGAAGGTTCGGG | 74 |

(5) Results

The primary chondrocytes obtained from the Runx2/Cbfa1-deficient mouse have a polygonal conformation that stores an extracellular matrix and a typical chondrocyte-like morphology (FIG. 1). The RU-1 chondrocyte cell line has morphology similar to that of the primary chondrocyte obtained from a Runx2/Cbfa 1-deficient mouse. In contrast, the RU-22 chondrocyte cell line has a very flat configuration with a low extracellular matrix expression level, and it does not have chondrocyte-like morphology. The expression levels of type II collagen are high both in the RU-1 and in RU-22 chondrocyte cell lines; however, the expression level of type X collagen is very low therein (FIG. 2). Accordingly, these cell lines are considered to maintain the properties of undifferentiated chondrocytes.

### Example 2

### Construction of a System of Inducing Cartilage Differentiation via Forced Expression of Runx2/Cbfa1 Using an Adenovirus

(1) Construction of an Adenovirus for the Expression of Runx2/Cbfa1

cDNA comprising the open reading frame (ORF) of murine Runx2/Cbfa1 was inserted into the BarnHI site of pIRES2-EGFP (Biosciences Clontech), and a fragment containing Runx2/Cbfa1, the internal ribosome entry site (IRES), and the enhanced green fluorescence protein (EGFP) was inserted into the BamHI-XbaI site of the shuttle vector pACCMV.pLpA (Proc. NatI. Acad. Sci. U.S.A., 1993, 90, 2812-2816). The constructed vector was cotransfected into the human kidney cell line 293 with the adenovirus vector pJMI7 (Virology, 1988, 163, 614-617) using the Superfect transfection reagent (Qiagen). The adenovirus retaining a Runx2/Cbfa1 fragment resulting via homologous recombination was subjected to subculture 3 or 4 times with the use of the cell line 293 to multiply the viruses. A roughly purified stock solution of multiplied viruses was purified via cesium chloride density-gradient ultracentrifugation and used as a virus stock solution for infection experiment. A recombinant adenovirus only retaining IRES-EGFP was also produced in the same manner as a control virus.

(2) Construction of a System for Inducing Cartilage Differentiation from Runx2/Cbfa1−/− and p53−/− Chondrocyte Cell Lines (RU-1 and RU-22) and Runx2/Cbfa1−/− primary Cultured Chondrocytes

The Runx2/Cbfa1−/− and p53−/− chondrocyte cell lines (RU-1 and RU-22) were plated onto a 24-well collagen-coated dish comprising a DMEM medium containing 10% fetal bovine serum and they were cultured to be confluent. Thereafter, cells were infected with the adenovirus for the expression of Runx2/Cbfa1 and the control adenovirus (for the expression of EGFP only) for 15 hours, the medium was exchanged with fresh medium, and culture was continued in the presence or absence of BMP-2 until day 15 while exchanging media every 3 or 4 days. The day of infection was determined to be day 0, and sampling was carried out at days 1, 3, 7, 11, and 15 for RNA preparation.

The Runx2/Cbfa1−/− primary cultured chondrocytes were plated onto a 24-well collagen-coated dish containing Dulbecco's Modified Eagle's Medium/Ham's F12 (1:1) hybrid medium (Gibco BRL) containing 10% fetal bovine serum, and they were cultured to be confluent. Thereafter, cells were infected with the adenovirus for the expression of Runx2/Cbfa1 and the control adenovirus (for the expression of EGFP only) for 15 hours, the medium was exchanged with fresh medium, and culture was continued in the presence or absence of BMP-2 until day 15 while exchanging media every 3 or 4 days. The day of infection was determined to be day 0, and sampling was carried out at days 1, 3, 7, 11, and 15 for RNA preparation.

cDNA was prepared from the sample for RNA preparation in the same manner as in Example 1 (4), and expression patterns of Runx2/Cbfa1, collagenase-3 (MMP-13), alkaline phosphatase (ALP), bone sialoprotein (BSP), a parathyroid hormone receptor (PTH/PTHrPR), type X collagen, osteopontin (OSP), and osteocalcin (OC) were assayed via real-time PCR analysis. The sequences of the primers used are shown in Table 2.

(3) Results and Discussion

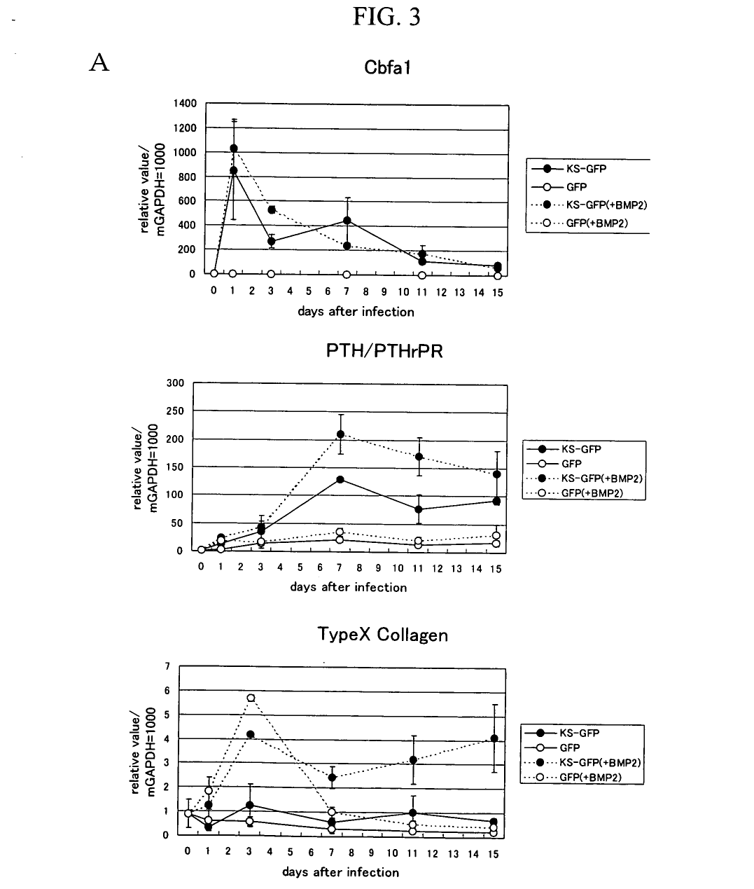

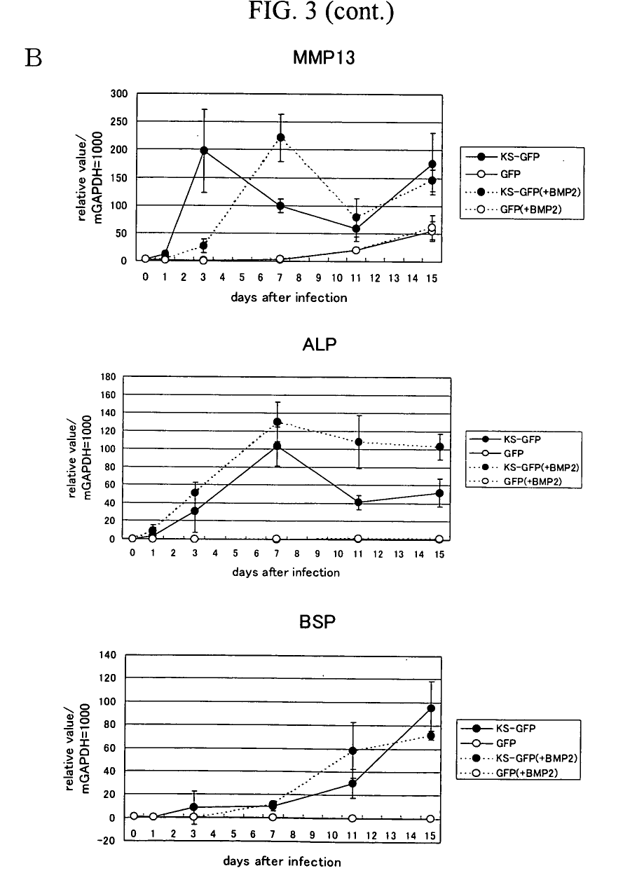

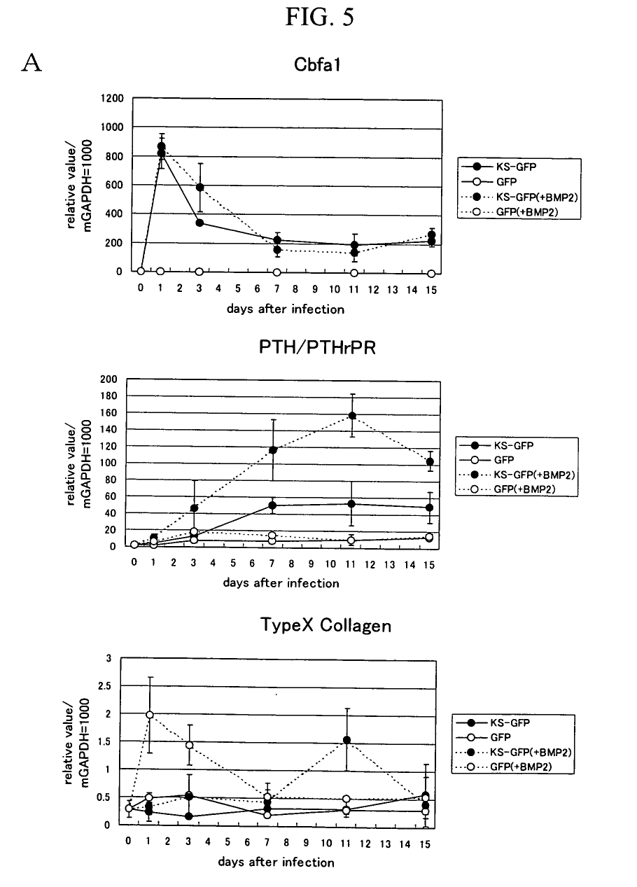

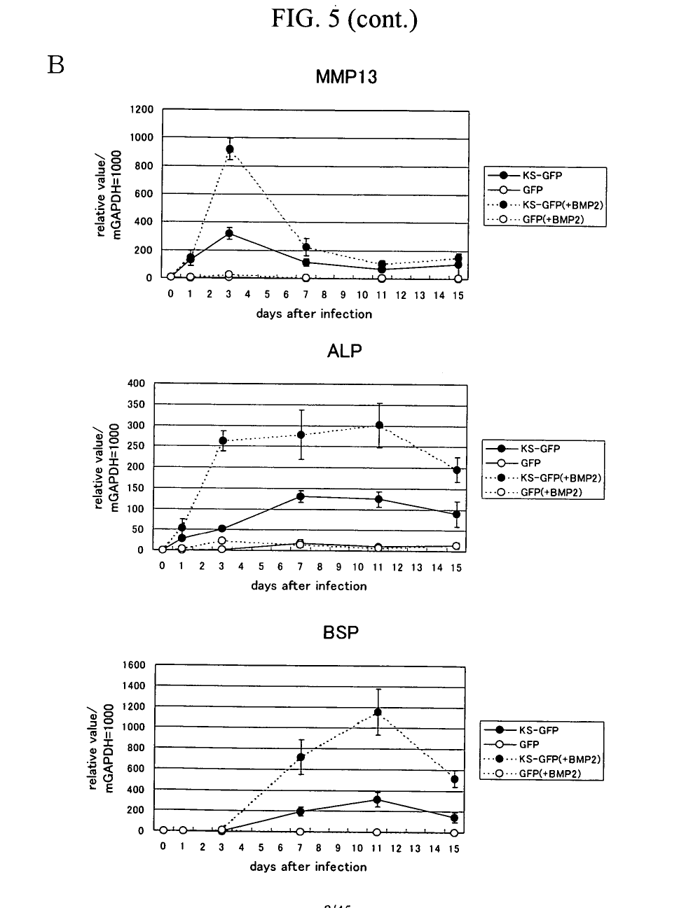

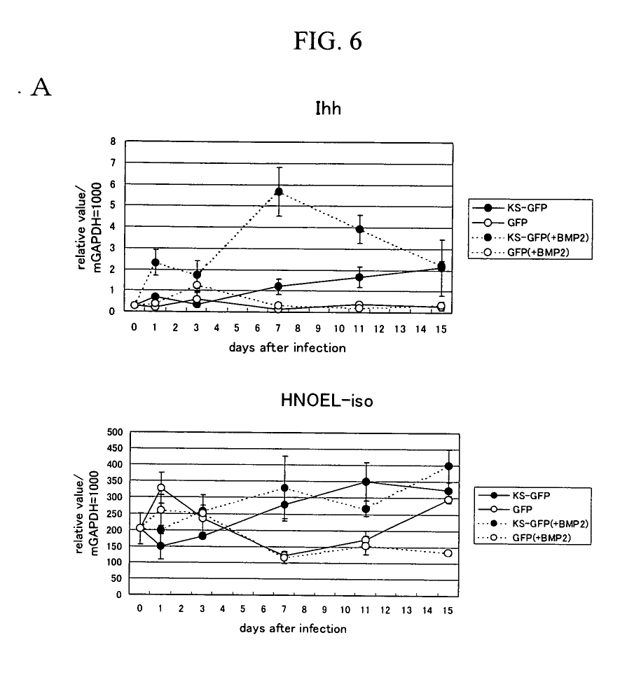

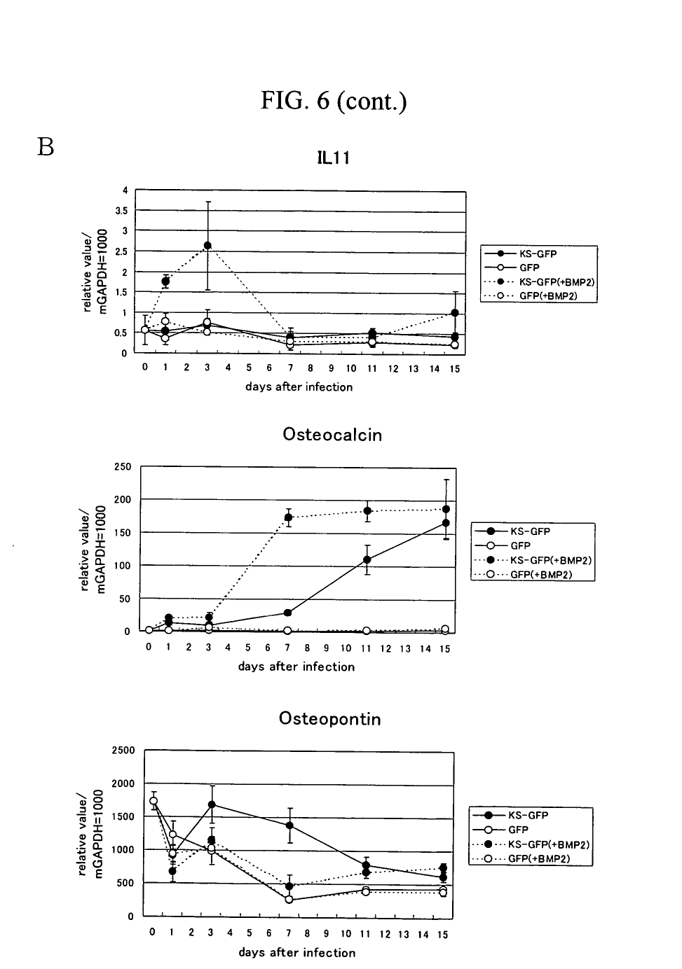

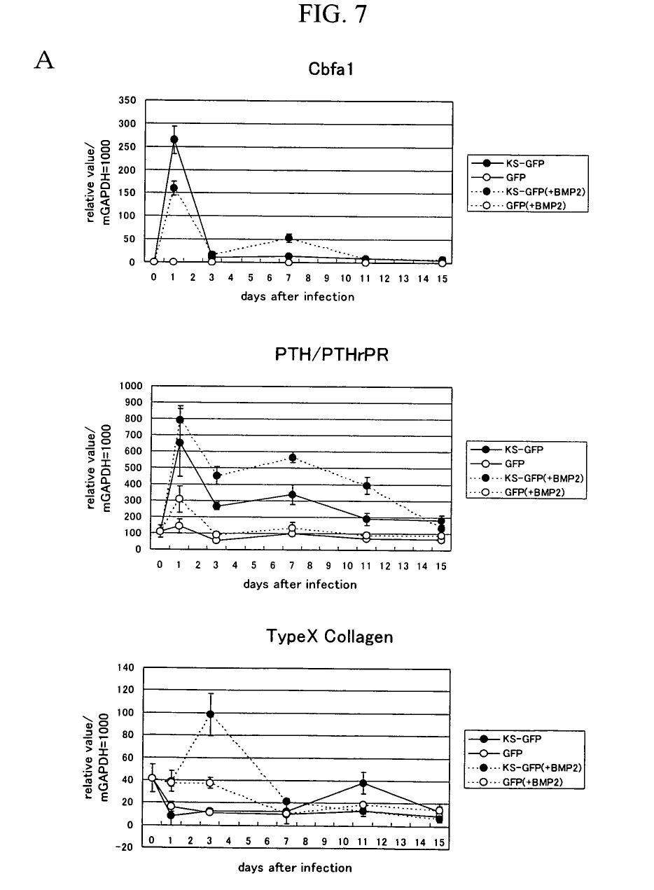

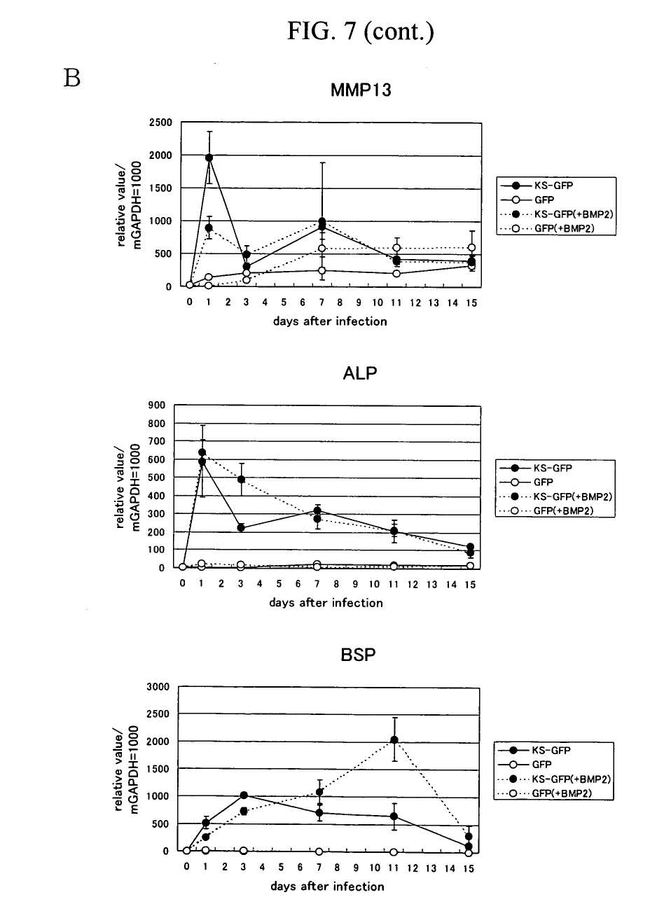

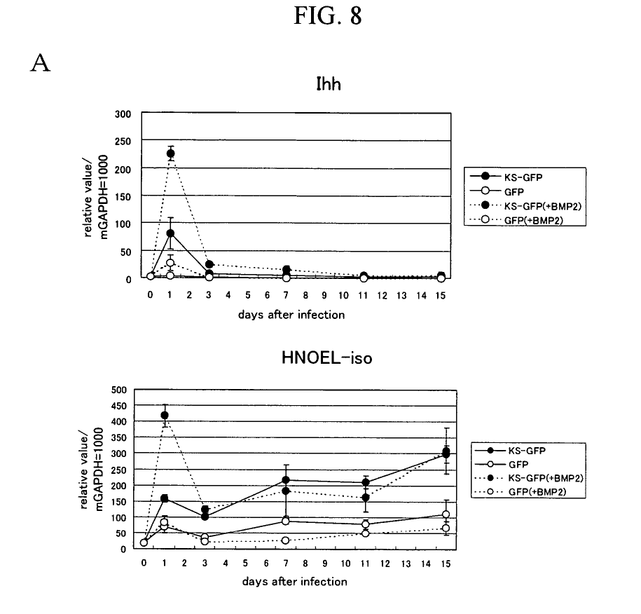

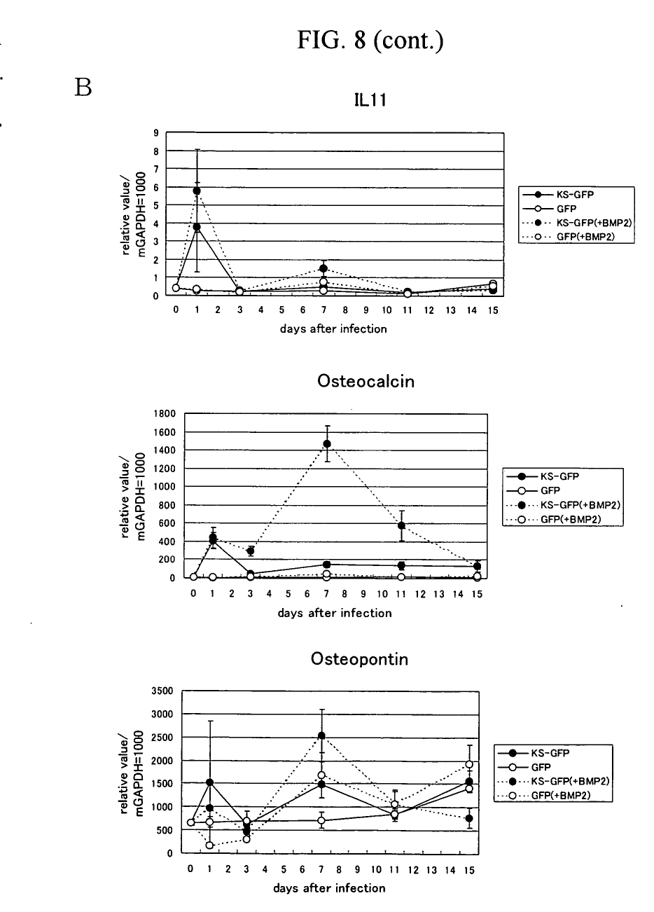

The results of real-time PCR analysis are shown in FIG. 3 to FIG. 8. The peak of Runx2/Cbfa1 expression was at day 1 in all the cell lines and primary cultured cells, and the expression level of Runx2/Cbfa1 was gradually lowered while maintaining a given level on day 3 and thereafter. The expression of a marker of a hypertrophic cartilage, i.e., type X collagen, was not induced along with the induction of Runx2/Cbfa1 expression, although the expression of an initial marker of a hypertrophic cartilage, i.e., PTH/PTHrPR, was induced in all the cell lines and primary cultured cells. This indicates that Runx2/Cbfa1 induces cartilage differentiation. The expressions of MMP-13, ALP, BSP, OSP, and OC, which are known downstream genes of Runx2/Cbfa1, were strongly induced by Runx2/Cbfa1, except for OSP. It is known that the expression of OSP is induced by serum alone, and thus, Runx2/Cbfa1-induced expression was considered to be less likely to occur in the in vitro culture system. Thus, this experimental system was found to be very useful for searching for the downstream gene of Runx2/Cbfa1.

cDNA was prepared from the sample for RNA preparation in the same manner as in Example 1 (4), and expression patterns of Runx2/Cbfa1, collagenase-3 (MMP-13), a parathyroid hormone receptor (PTH/PTHrPR), alkaline phosphatase (ALP), type X collagen, bone sialoprotein (BSP), Indian hedgehog (Ihh), Interleukin-11 (IL-11), HNOEL-iso, osteocalcin (OC), and osteopontin (OSP) were assayed via real-time PCR analysis. The sequences of the primers used are shown in Tables 2 and 3. The expression of Runx2/Cbfa1 was assayed in order to confirm the expressions of the genes derived from the Runx2/Cbfa1-infected viruses. MMPI3, ALP, BSP, Ihh, IL-11, OC, and OSP are known downstream genes the expressions of which are induced by Runx2. If the expressions of such genes are induced, it becomes possible to verify that Runx2 derived from infecting viruses performs successfully and that cartilage differentiation is stimulated. The expression level of PTH/PTHrPR and that of type X collagen are known to increase along with cartilage differentiation. Accordingly, increased expression levels of such genes indicate that chondrocytes are differentiated. It should be noted that PTH/PTHrPR is expressed at a relatively early stage of cartilage differentiation and type X collagen is expressed at a later stage thereof HNOEL-iso was subjected to assay as a downstream gene of Runx2, which had been newly discovered by the present inventors.

(3) Results and Discussion

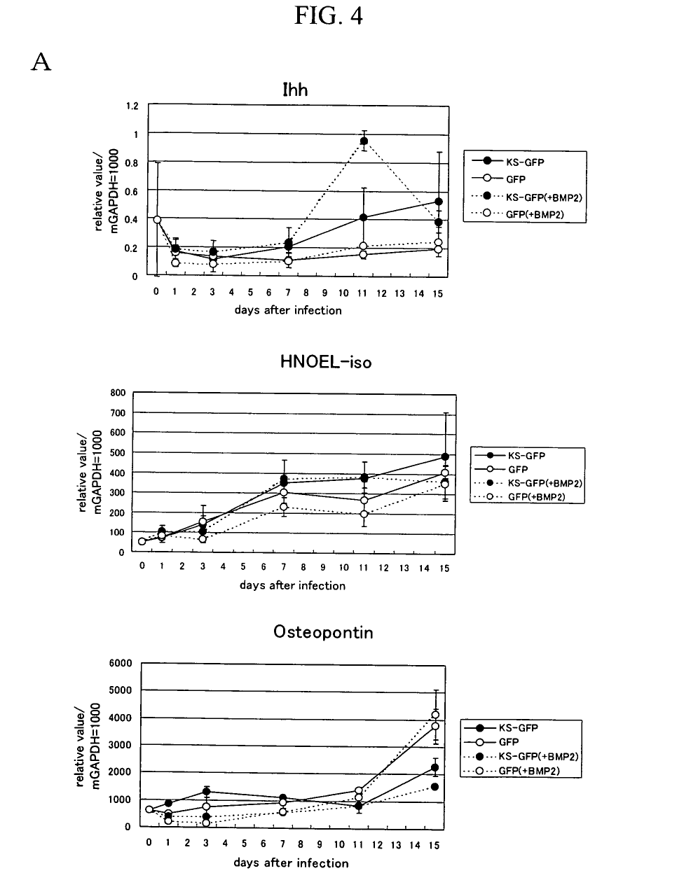

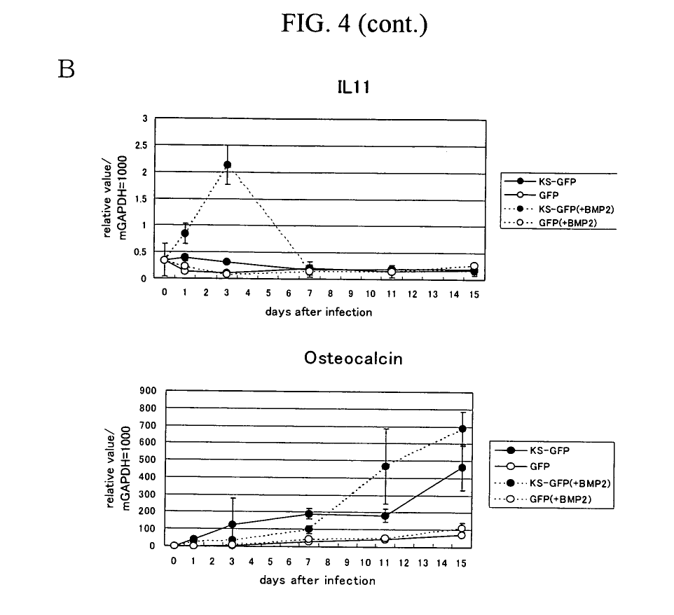

The results of real-time PCR analysis are shown in FIG. 3 to FIG. 8. The results of infection with an adenovirus for the expression of Runx2/Cbfa1 are represented by KS-GFP (black dots), and the results of infection with the control adenovirus (for the expression of EGFP only) are represented by GFP (white dots). The presence of BMP2 is represented by “+BMP2.” The peak of Runx2/Cbfa1 expression was at day 1 in all the cell lines and primary cultured cells, and the expression level of Runx2/Cbfa1 was gradually lowered while maintaining a given level on day 3 and thereafter (FIGS. 3A, 5A, 7A: Cbfa1). The expression of a marker of a hypertrophic cartilage, i.e., type X collagen, was not induced along with the induction of Runx2/Cbfa1 expression (FIGS. 3A, 5A, 7A: type X collagen), although the expression of an initial marker of a hypertrophic cartilage, i.e., PTH/PTHrPR, was induced in all the cell lines and primary cultured cells (FIGS. 3A, 5A, 7A: PTH/PTHrPR). This indicates that Runx2/Cbfa1 induces cartilage differentiation. The expressions of MMP-13, ALP, and BSP (FIGS. 3A, 5A, and 7A), OSP, and OC (FIGS. 4B, 6B, 8B), which are known downstream genes of Runx2/Cbfa1, were strongly induced by Runx2/Cbfa1, except for OSP. It is known that the expression of OSP is induced by serum alone, and thus, Runx2/Cbfa1-induced expression was considered to be less likely to occur in the in vitro culture system. Induction of such gene expression was enhanced in the presence of BMP2. A variety of growth factors including BMP2 are present in cartilage tissues in an organism, and they regulate proliferation and differentiation of chondrocytes. Runx2-induced gene expression was enhanced in the presence of BMP2. This indicates that the experimental system of the present invention involves in vivo cartilage differentiation. Thus, this experimental system was found to be very useful for searching for the downstream gene of Runx2/Cbfa 1.

### Example 3

### Isolation of Downstream Gene of Runx2/Cbfa1 via Subtraction

The downstream gene of Runx2/Cbfa1 was obtained via subtraction using the undifferentiated mesenchymal cell line (C3H10T1/2). At the outset, the C3H10T1/2 cell line that intensively expresses Runx2/Cbfa1 was established (C3H10T1/2-Runx2/Cbfa1). Subsequently, C3H10T1/2-Runx2/Cbfa1 and C3H10T1/2 were used to screen for the gene that is intensively expressed in the C3H10T1/2-Runx2/Cbfa1 cell line via subtraction. Subtraction was carried out using the CLONTECH PCR-Select™ cDNA Subtraction Kit in accordance with the user's manual. As a result, the expression level of the gene shown in SEQ ID NO: 11 (Riken cDNA 2810002E22 gene (HNOEL-iso homolog)) was found to be higher in C3H10T1/2-Runx2/Cbfa1 than in C3H10T1/2.

Whether or not the HNOEL-iso homolog gene is induced by Runx2/Cbfa1 was analyzed via real-time PCR in RU-1, RU-22, and Runx2/Cbfa1−/−mouse-derived primary cultured chondrocytes, in order to confirm that the gene is induced to be expressed by Runx2/Cbfa1. The primers used in the real-time PCR analysis are shown in Table 3. Analysis via real-time PCR was carried out in the same manner as in Example 1 (4). The results of the analysis are shown in FIGS. 4A, 6A, and 8A.

As a result, it was found that the gene of interest was strongly induced to be expressed by Runx2/Cbfa1 in RU-22 chondrocytes and in primary chondrocytes (FIGS. 4A, 6A, and 8A). Accordingly, the HNOEL-iso homolog gene was also found to be a downstream gene of Runx2/Cbfa1.

### Example 4

### DNA Microarray Analysis

(1) DNA Microarray Analysis Using RU-1 and RU-22 Chondrocyte Cell Lines

The RU-1 and RU-22 cell lines were plated onto 10 wells of the 12-well collagen-coated plate, they were cultured to be confluent, and they were then infected with the adenovirus for the expression of Runx2/Cbfa1 and the control virus (for the expression of GFP only). One day after the injection, total RNA was recovered using Isogen 1 and total RNA was prepared in accordance with the instructions of the reagent. Thereafter, poly A+ RNA was prepared using the Oligotex-dT30<Super>mRNA Purification Kit (Takara) in accordance with the instructions of the kit, and the resultant was employed as a sample for DNA microarray analysis. DNA microarray analysis was carried out using LifeArray (Kurabo Industries Ltd.; the number of mouse genes: approximately 9,500).

(2) DNA Microarray Analysis Using Primary Cultured Chondrocytes Derived from Runx2/Cbfa1 −/−

The primary cultured chondrocytes derived from Runx2/Cbfa1−/− were plated onto 10 wells of the 12-well collagen-coated plate, they were cultured to be confluent, and they were then infected with the adenovirus for the expression of Runx2/Cbfa1 and the control virus (for the expression of GFP only). One day after the injection, total RNA was recovered using Isogen I and total RNA was prepared in accordance with the instructions of the reagent. The resultant was employed as a sample for DNA microarray analysis. DNA microarray analysis was carried out using the CodeLink DNA microarray (Kurabo Industries Ltd.; the number of mouse genes: approximately 10,000).

(3) Results

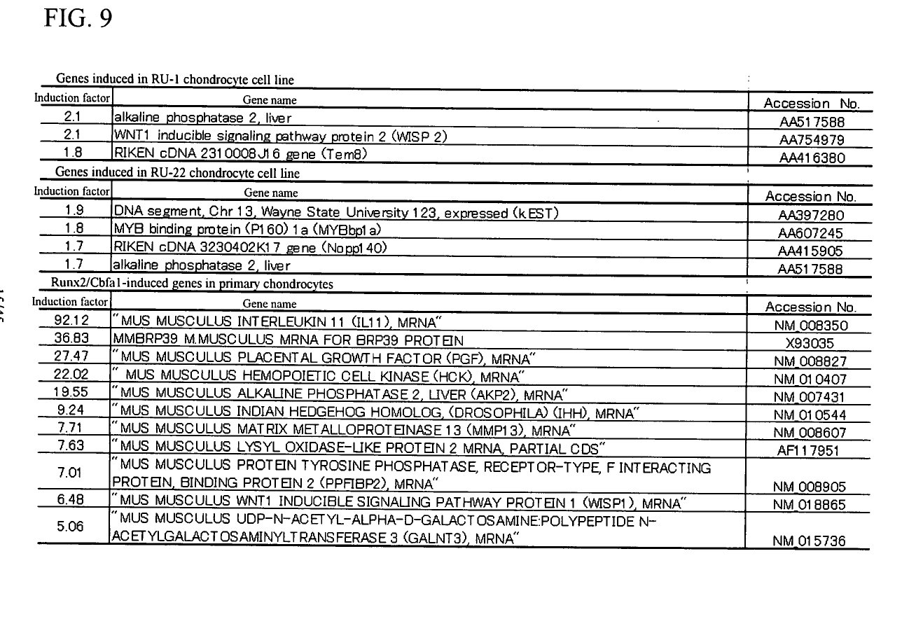

FIG. 9 shows some genes the expressions of which are induced along with the forced expression of Runx2/Cbfa1, which were identified via DNA microarray analysis. With the use of the LifeArray, expression of alkaline phosphatase (ALP), which is known to be regulated by Runx2/Cbfa1, was found to be induced in each cell line and in primary cultured cells. This indicates that the performance of the experimental system is successful. With the use of the CodeLink DNA microarray, the expression of alkaline phosphatase (ALP) and that of collagenase-3 (MMP-13), which are known to be induced by Runx2/Cbfa1, were found to be induced. This indicates that the performance of the experimental system is successful.

### Example 5

### Determination of cDNA Sequence of KEST Gene

cDNA was synthesized based on RNA obtained from the skeletal tissue of a mouse embryo at day 15 of embryonic development using the SMART™ RACE cDNA amplification kit (ClonTech) in accordance with the manufacturer's instructions. Primers were synthesized based on the partial nucleotide sequence of the mouse kEST (AA397280), and cDNA was amplified by the RACE method in accordance with the manufacturer's instructions. The resulting PCR product was electrophoresed on 1% agarose gel containing ethidium bromide, and the gel was observed under ultraviolet light to detect a DNA band. The amplified fragment was removed from the gel and purified using the QlAquick Gel Extraction Kit (Qiagen) in accordance with the manufacturer's instructions. The nucleotide sequence of the purified fragment was determined using the DNA sequencer (ABI PRISM™ 310 Genetic Analyzer, PE Applied Biosystems) and the ABI PRISM™ BigDye Terminator Cycle Sequencing Ready Reaction kit (PE Applied Biosystems) in accordance with the manufacturer's instructions.

The nucleotide sequence of the cDNA of the mouse kEST and the amino acid sequence deduced therefrom are shown in SEQ ID NO: 9.

### Example 6

### Confirmation of Runx2/Cbfa1-Induced Expression via Real-Time PCR Analysis

As a result of DNA microarray analysis using the established cell lines and the primary cultured cells, the genes the expressions of which are induced by Runx2/Cbfa1 in chondrocytes were identified. Subsequently, whether or not these genes were induced to be expressed by Runx2/Cbfa1 with reproducibility was examined via real-time PCR analysis employing the samples of Example 2 (2) (days 0, 1, and 3) as the templates. Runx2/Cbfa1-induced expression was confirmed with the use of the cell lines or primary cultured cells in which such gene expression was found to be induced via DNA microarray analysis. Real-time PCR analysis was carried out in the same manner as in Example 1 (4). Primers used for such expression analysis are shown in Table 3.

TABLE 3
List of primers used for real-time PCR analysis (2)

| | | |
|---|---|---|
| | | SEQ |
| | | ID |
| Murine genes | Primer sequences | NO: |

| | | | |
|---|---|---|---|
| Tumor endothelial marker 8 | Forward | TGTGACAGCCAGCTCGAAAAC | 75 |
| precursor (Tem8) | Reverse | TGGAGAGCCAAGACTTTTCCA | 76 |
| WNT1 inducible signaling | Forward | GCCTGCCATTCTCAGCAAA | 77 |
| pathway protein 2 (Wisp2) | Reverse | ACACTGAATCCACCCAGGACA | 78 |
| Nucleolar and coiled-body | Forward | CAAGTCCTTCCGGCATGAA | 79 |
| phosphoprotein 1 (Nolc1) | Reverse | CCAAAATCACCCTTCCTTTGC | 80 |
| (Nopp140) | | | |
| MYB binding protein (P160) | Forward | CTTGACAACACAGCAGCGTCA | 81 |
| 1a (Mybbp1a) | Reverse | ACTTGAAGATGTGGAGGCCCA | 82 |
| DNA segment, Chr 13, Wayne | Forward | TGGTCAAGAAAGCACCAATGC | 83 |
| State University 123, | Reverse | CCACACACGCCATCTTTCTTC | 84 |
| expressed (k. EST) | | | |
| RIKEN cDNA 2810002E22 gene | Forward | TTGTCTATAACACCCGCCCTG | 85 |
| (HNOEL-iso homolog) | Reverse | CGGCGTGGAAAATAGGAGAGT | 86 |
| BRP39 | Forward | AGGAGAAGAAGCTGGCAGGAG | 87 |
| | Reverse | CTTGATGGCGTTGGTGAGC | 88 |
| Hemopoietic cell kinase (HCK) | Forward | TGGGTACCGTATGCCTCGA | 89 |
| | Reverse | TGTATTCAAAGGTGGGCCG | 90 |
| Lysyl oxidase-like 2 (LOXL2) | Forward | TGCTGGGACATGTACCGTCAT | 91 |
| | Reverse | CCAGATGCGGTAGCCATCA | 92 |
| Protein tyrosine phosphatase, | Forward | GCCTTCTCTGTGCACGGAG | |
| receptor-type, F interacting | Reverse | GTGAGACCAGCCCAAAGACAC | 94 |
| protein, binding protein 2 | | | |
| (PPFIBP2) | | | |
| WNT1 inducible signaling | Forward | ATGGGAGTTGGTAGGGACCG | 95 |
| pathway protein 1 (Wisp 1) | Reverse | CATGGGAGGGTGATCCACTT | 96 |
| Placental growth factor (P1GF) | Forward | GGCTGCATTGAAGGCATGT | 97 |
| | Reverse | AAGGGCAAACTCCACAGGC | 98 |
| UDP-N-acetyl-alpha-D- | Forward | TTACCCGGAAGCGTATGTGC | 99 |
| galactosamine: polypeptide | | | |
| N-acetylgalactosaminyl- | | | |
| transferase 3 (GALNT3) | | | |

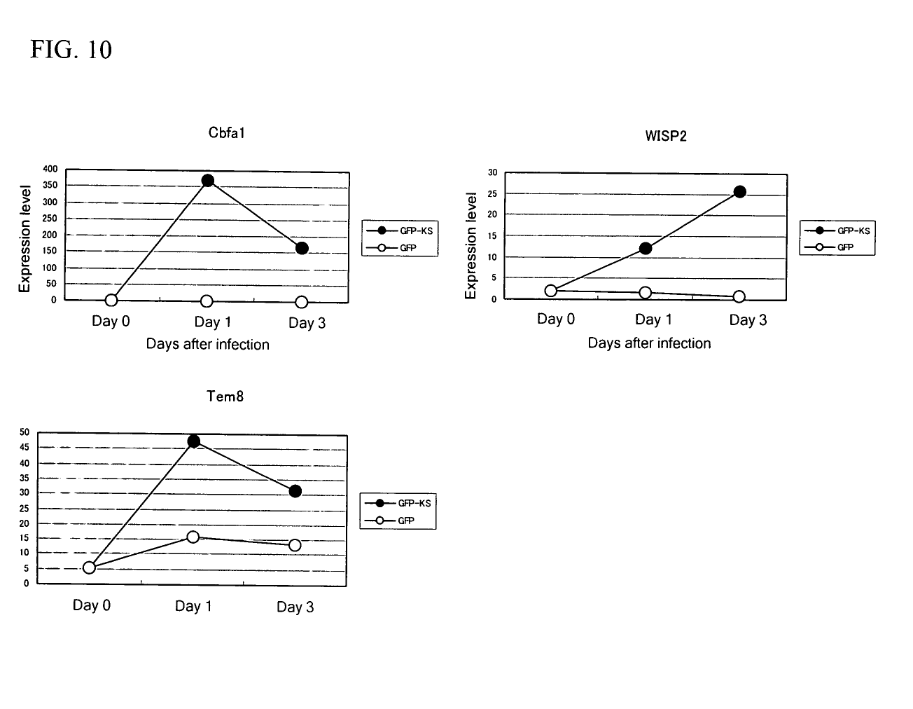

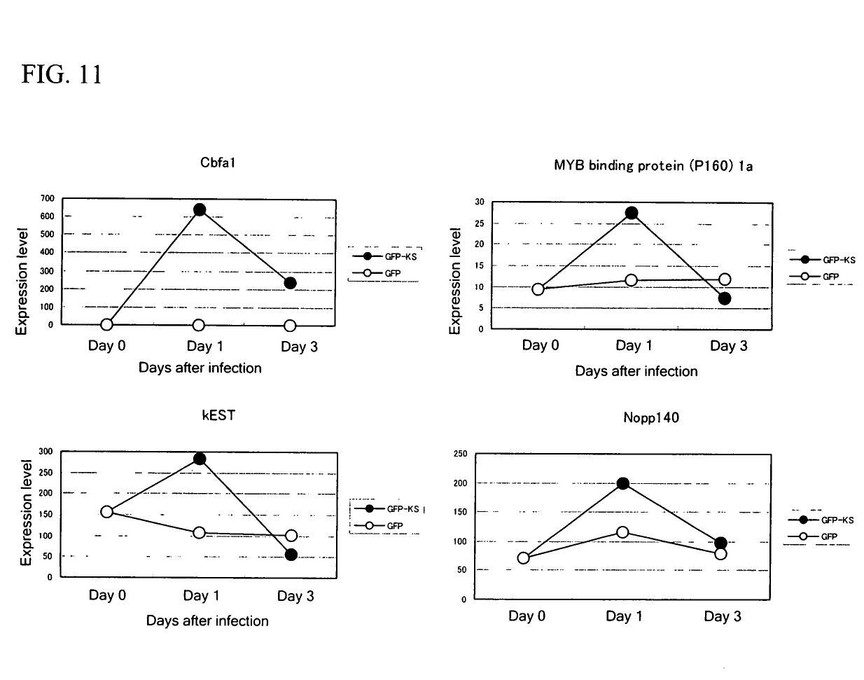

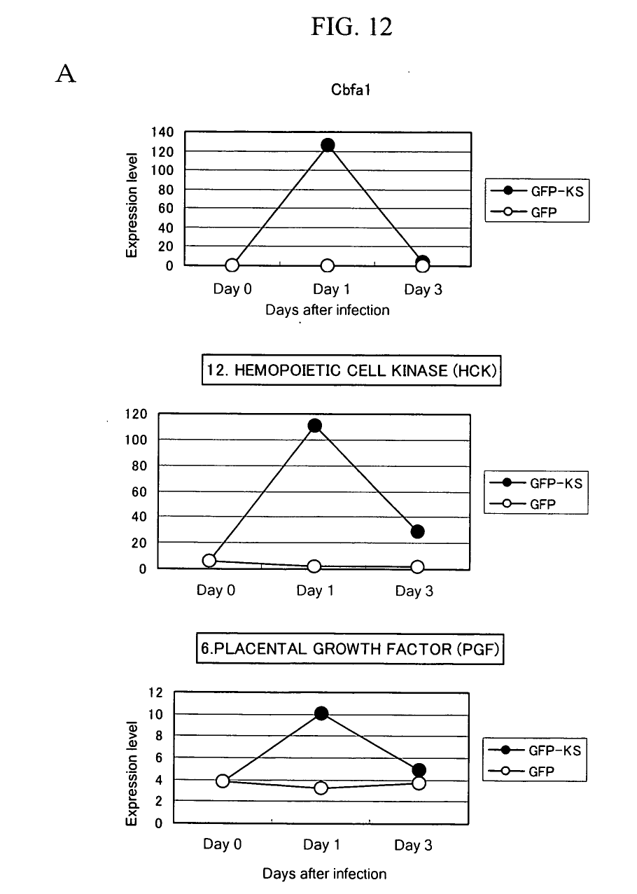

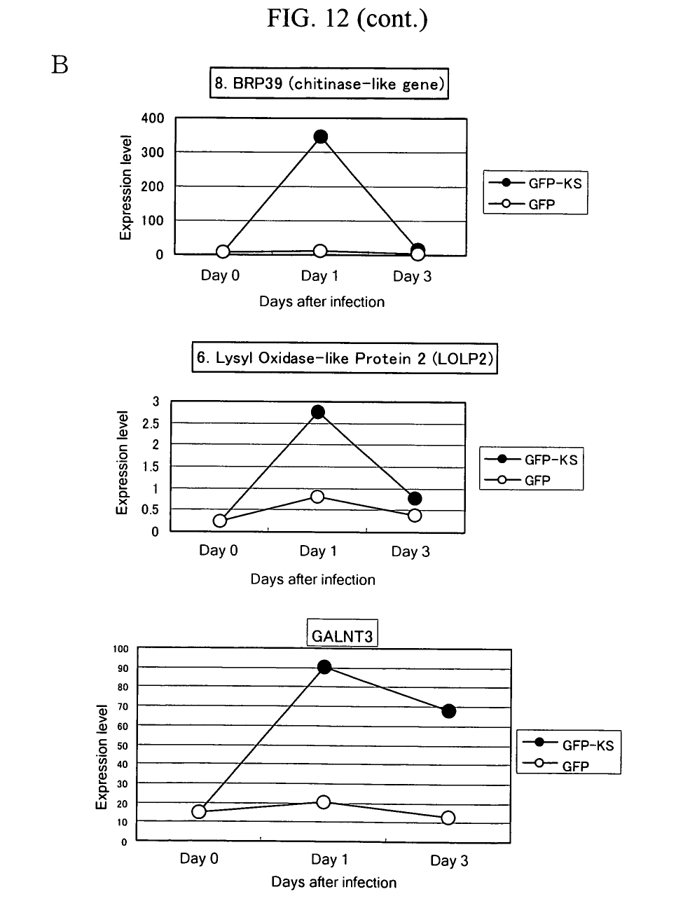

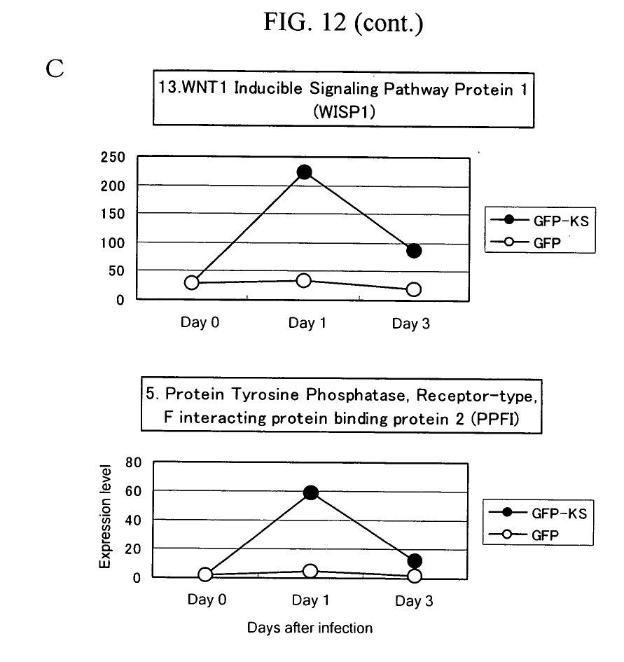

As a result, it was found that all the genes were induced to be expressed by Runx2/Cbfa1, and the results of the real-time PCR analysis were the same as those obtained via DNA microarray analysis (FIGS. 10 to 12).

### Example 7

### Expression Analysis of Downstream Gene of Runx2/Cbfa1 in Embryonic Skeletons of Wild-Type Mouse and Runx2/Cbfa1-Deficient Mouse

The gene the expression of which was verified to be induced by Runx2/Cbfa1 via DNA microarray analysis was analyzed concerning the expression thereof in the embryonic skeleton of a wild-type mouse and of a Runx2-Cbfa1-deficient mouse. At the outset, total RNAs were prepared from the skeleton of a wild-type mouse at days 13.5, 15.5, and 18.5 of embryonic development and from that of a Runx2/Cbfa1-deficient mouse at day 18.5 of embryonic development. cDNAs were prepared therefrom. Total RNA and cDNA were prepared in the same manner as in Example 1 (4). Subsequently, 12 genes the expressions of which were found to be induced by Runx2/Cbfa1 via DNA microarray analysis were subjected to expression analysis in the embryonic skeletons via real-time PCR. Real-time PCR analysis was carried out in the same manner as in Example 1 (4).

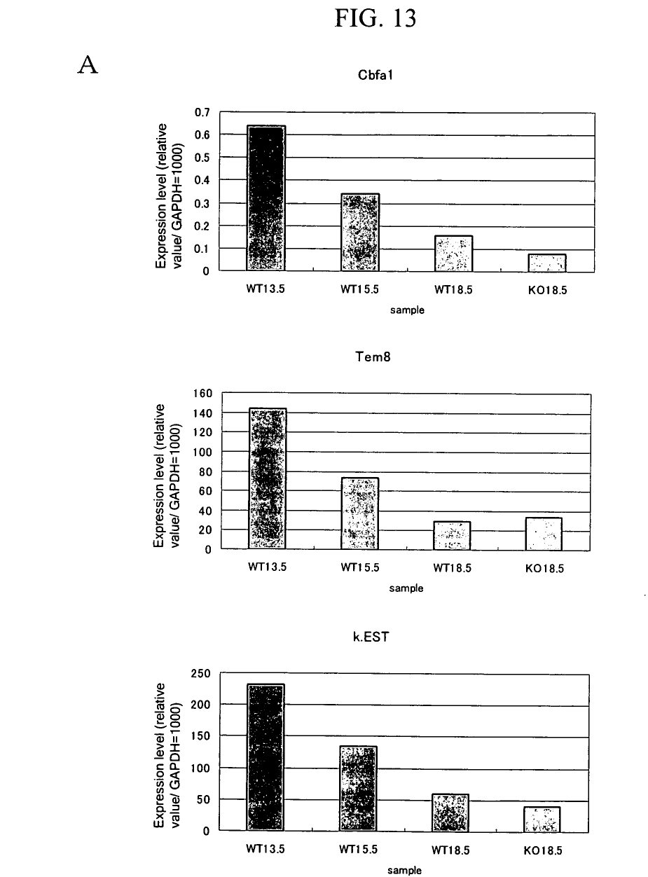

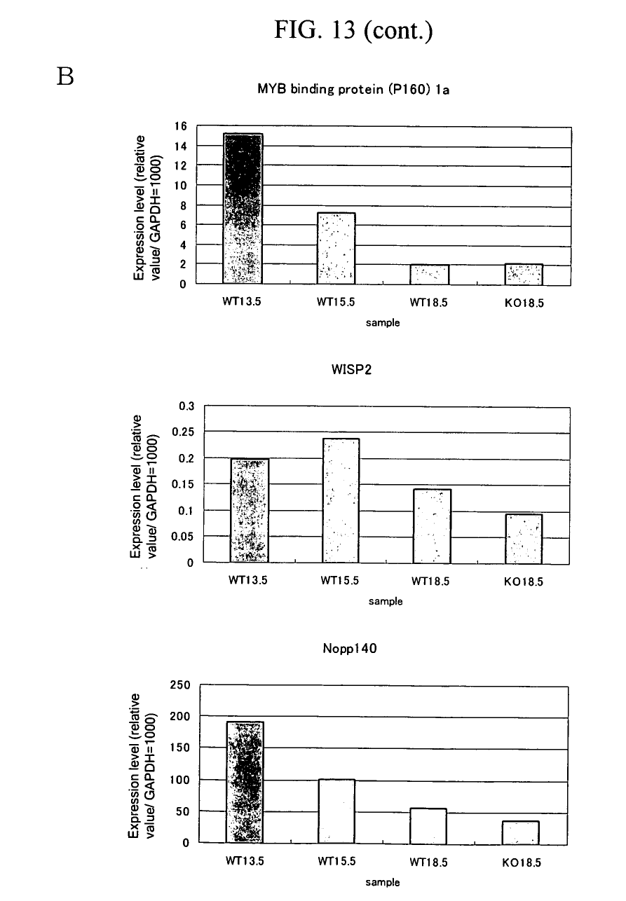

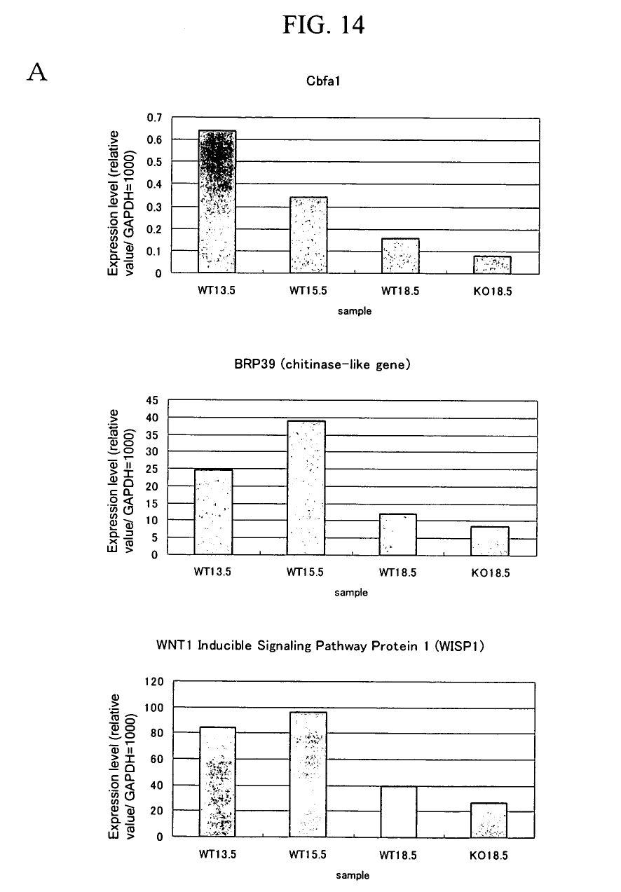

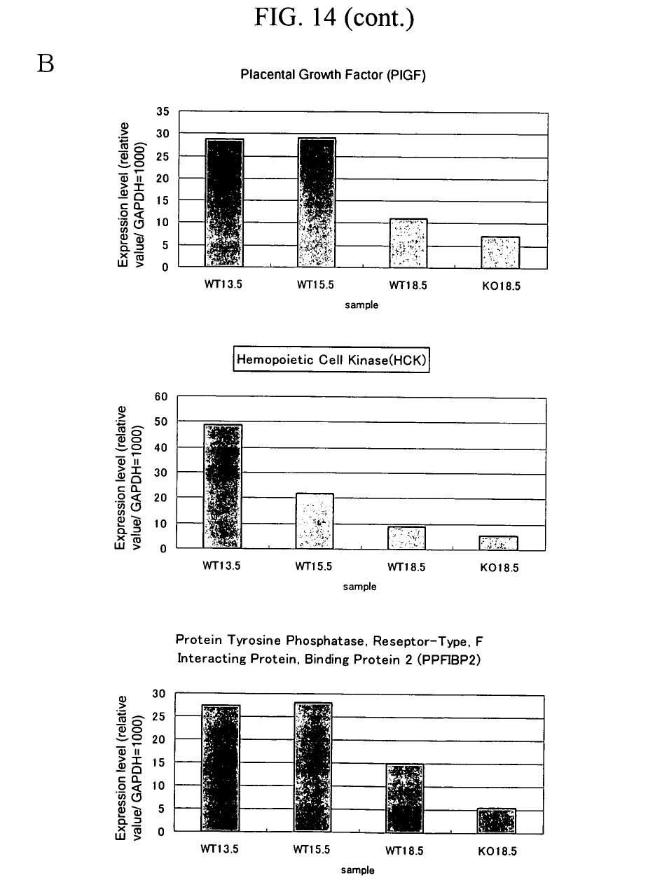

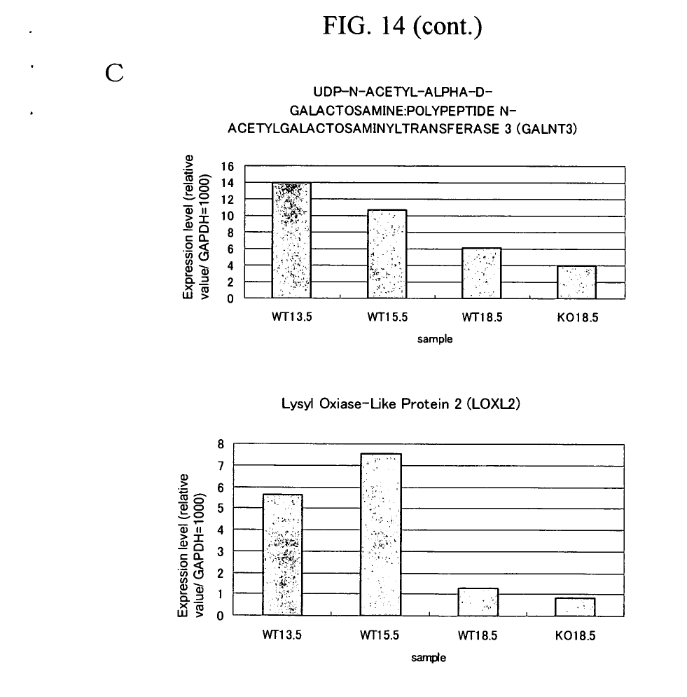

As a result, expressions of all these 13 genes were found to be inhibited in the Runx2/Cbfa1-deficient mouse at day 18.5 of embryonic development to a greater extent than in the wild-type mouse at days 13.5 and 15.5 of embryonic development (FIGS. 13A and 13B and FIGS. 14A, 14B, and 14C). The skeletal differentiation was significantly delayed in the Runx2/Cbfa1-deficient mouse, and the skeleton of the Runx2/Cbfa1-deficient mouse at day 18.5 of the embryonic development is equivalent to that of a wild-type mouse at days 13.5 and 15.5 of embryonic development. This indicates that the expressions of all these 13 genes were inhibited in the skeleton of the Runx2/Cbfa1 deficient mouse and that such expressions are inhibited by Runx2/Cbfa1.

### Example 8

### Generation of Wisp2 Transgenic Mouse Using Yype II Collagen Promoter and Examination of its Functions in Chondrocytes

A transgenic mouse, which allows cartilage-specific expression of the mouse Wisp2 gene shown in SEQ ID NO: 3 with the aid of a type II collagen promoter, was generated (a Wisp2 transgenic mouse), and functions in chondrocytes were examined.

(1) Preparation of a Construct for Transgenic Mouse

The mouse Wisp2 genes were amplified via PCR using the following forward primer (F) and the reverse primer (R) and using the template cDNA obtained from the skeleton of a wild-type mouse at day 13.5 of embryonic development.

| | |
|---|---|
| <Wisp2> | |
| F: 5′ GCG GCC GCA CCA TGA GGG GCA ACC CAC TGA TC 3′ | (SEQ ID NO: 101) |
| R: 5′ GCG GCC GCC TAG AAG GCA CTG TTC CAT GA 3′ | (SEQ ID NO: 102) |

The Col2al-based expression vector reported by Ueda et al. was employed for the transgenic mouse (J. Cell. Biol., 2001, 153, 87-99). This vector comprises a murine type II collagen promoter and an enhancer, and it has been confirmed to be expressed in a cartilage-specific manner. The obtained PCR fragment was incorporated into the NotI site ofthe Col2al-based expression vector, and the resultant was employed as a construct for preparing a transgenic mouse.

(2) Generation of Transgenic Mouse

The construct for generating a transgenic mouse was treated with Narl to cleave a fragment containing a type II collagen promoter, the gene to be expressed (Wisp2), and an enhancer. The fragment was purified on agarose gel and then injected into the nucleus of the fertilized egg obtained from the F1 hybrid mouse (C57BL/6×C3H). The fertilized egg that had experienced injection was introduced into the uterus of the foster mother, the embryo was removed via Caesarean operation, the genome was extracted from the liver, and the incorporation of the fragment into the genome was confirmed via PCR. Also, total RNA was extracted from the upper-body skeleton, cDNA was synthesized, and the expression intensity was assayed via real-time PCR analysis.

(3) Preparation, Staining, and In situ Hybridization of Skeleton Section

The lower-body skeleton was sliced, and the section was observed under an optical microscope. The lower-body skeleton of the fetus was fixed in 4% paraformaldehyde in 0.1M phosphate buffer. Thereafter, a 7-μm section was prepared and stained with hematoxylin and eosin (i.e., HE staining). Type II collagen (Col2al), PTH receptor-1 (Pthr1), type X collagen (Col10al), and osteopontin were subjected to in silt hybridization in accordance with a conventional technique using conventional probes (Dev Dyn. April 1999, 214 (4), 279-90).

(4) Results and Discussion

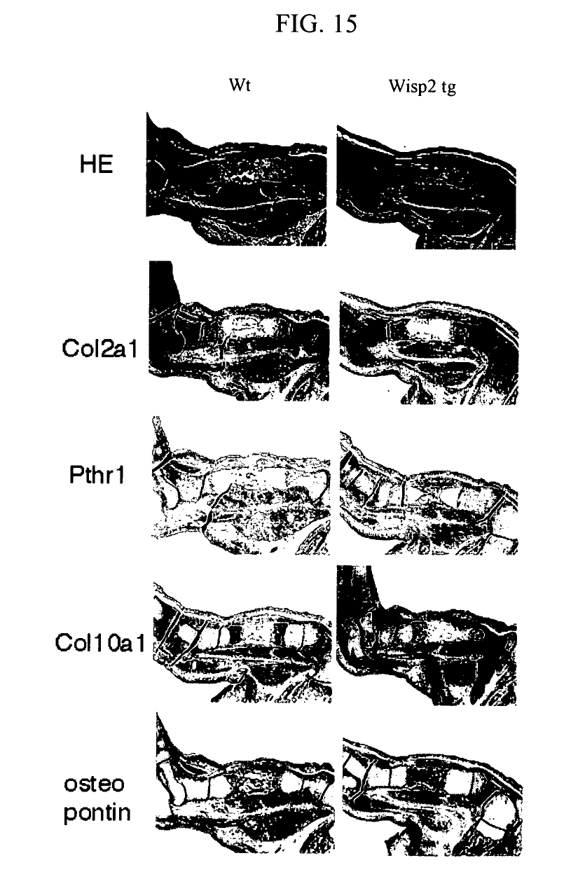

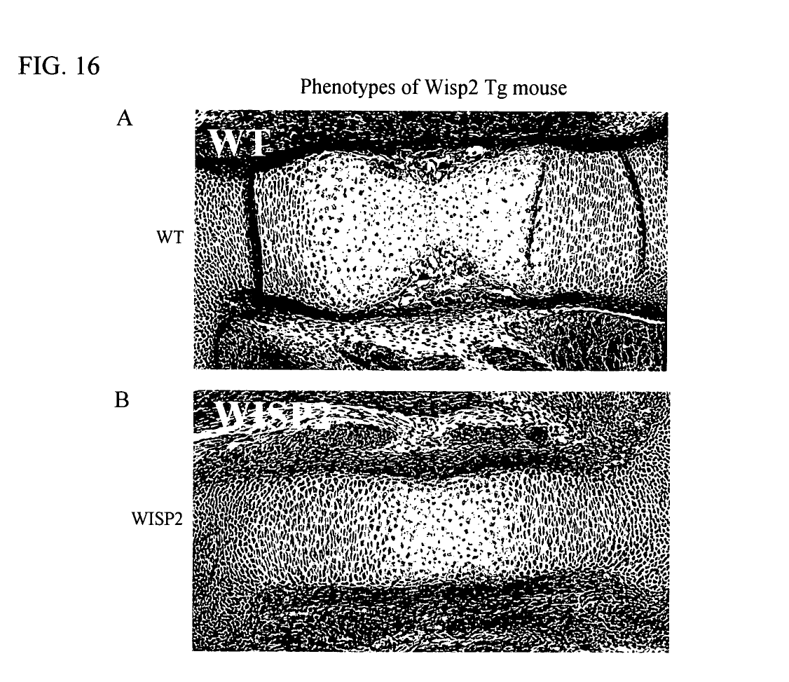

The Wisp2 transgenic mouse was slightly smaller than the wild-type mouse. Expressions of Col2al, Pthr1, Col10al, and osteopontin at day 16.5 of embryonic development were analyzed via in situ hybridization, and the results thereof are shown in FIG. 15. Between the wild-type mouse (Wt) and the Wisp2 transgenic mouse (Wisp2 tg), there was no significant difference in such gene expression. FIG. 16A and 16B show images of the HE-stained skeleton sections of a wild-type mouse and of a Wisp2 transgenic mouse at day 16.5 of embryonic development. In the case of the wild-type mouse (WT; A), a blood vessel had already infiltrated the cartilage. In the case of the Wisp2 transgenic mouse (WISP2; B), a bone collar was formed. However, the hypertrophied layer was shorter compared with the wild-type, blood vessel infiltration was not observed, and cartilage differentiation and enchondral ossification were delayed. It was accordingly considered that the Wisp2 gene inhibited cartilage differentiation.

### Example 9

### Generation of Nopp140, Tem8, HCK, and GALNT3 Transgenic Mice Using Type II Collagen Promoter and Analysis of their Functions in Cartilage

Transgenic mice, which allow cartilage-specific expression of the murine Nopp140 gene shown in SEQ ID NO: 5, the murine Tem8 gene shown in SEQ ID NO: 1, the HCK gene shown in SEQ ID NO: 15, and the GALNT3 gene shown in SEQ ID NO: 25 under the control of a type II collagen promoter, were prepared, and functions in chondrocytes were further examined.

(1) Preparation of a Construct for a Transgenic Mouse

The murine Nopp140 gene, the murine Tem8 gene, the murine HCK gene, and the murine GALNT3 gene were amplified via PCR using the following forward primers (F) and the reverse primers (R). cDNA obtained from the skeleton of a wild-type mouse at day 13.5 of embryonic development was used as a template concerning the murine Nopp140 gene and the murine Tem8 gene. cDNA obtained from the skeleton of a wild-type mouse at day 15.5 of embryonic development was used as a template concerning the murine HCK gene and the murine GALNT3 gene. A constitutive active form of HCK (499Tyr-Phe) was prepared in accordance with the previous report (J. Exp. Med., 2002, Vol. 196, No. 5, 589-604).

| | |
|---|---|
| <Nopp140> | |
| F: 5′-GAC GCG TTG CGG CCG CAG CAT GGC GGA TAC CGG CTT-3′ | (SEQ ID NO: 103) |
| R: 5′-AAA GGA TGG CGG CCG CTC ACT CGC TGT CGA ATT TGA-3′ | (SEQ ID NO: 104) |
| <Tem8> | |
| F: 5′-GCG GCC GCA GCA TGG ACC GCG CGG GGC GC-3′ | (SEQ ID NO: 105) |
| R: 5′-ATG CAT CTA GAC AGA AGG CCT TGG AGG AG-3′ | (SEQ ID NO: 106) |
| <HCK> | |
| F: 5′-GCG GCC GCA GGA TGG GAT GCG TGA AGT CCA GG-3′ | (SEQ ID NO: 107) |
| R: 5′-GCG GCC GCT CAA GGC TGC TGC TGG AAC TGG CTC TCA GTG GCC GT-3′ | (SEQ ID NO: 108) |
| <GALNT3> | |
| F: 5′-GCG GCC GCA GAA TGG CTC ACC TTA AGG GAC TA-3′ | (SEQ ID NO: 109) |
| R: 5′-GCG GCC GCT TAT TCA TTT TGG CTA AAA ATC CA-3′ | (SEQ ID NO: 110) |

A vector for a transgenic mouse of each gene was prepared in the same manner as in Example 8 using the Col2al-based expression vector described in Example 8.

(2) Generation of a Transgenic Mouse

The constructs for generating transgenic mice of the Nopp140, HCK, and GALNT3 genes were treated with NarI and the construct for generating transgenic mice of the Tem8 gene was treated with PvtII to cleave the fragments comprising type II collagen promoters, the genes to be expressed, and the enhancers. The fragments were purified using agarose gel and then injected into the nuclei of the fertilized eggs obtained from the F1 hybrid mouse (C57BL/6×C3H). The fertilized eggs that had experienced injection were introduced into the uterus of the foster mother, the fetuses were removed via Caesarean operation, the genomes were extracted from the liver, and the incorporation of the fragments.into the genomes was confirmed by PCR. Also, total RNAs were extracted from the upper-body skeletons, cDNAs were synthesized, and expression intensity was assayed via real-time PCR analysis. The skeletons of the fetuses were stained with alizarin red and with alcian blue (Cell, 1997, 89, 755-764).

(3) Preparation of Skeleton Sections

The lower-body skeletons were sliced, and the sections were observed under an optical microscope. The lower-body skeletons of the fetuses were fixed in 4% paraformaldehyde in 0.1M phosphate buffer. Thereafter, 7-μm sections were prepared and stained with hematoxylin and eosin (i.e., HE staining) to analyze the skeletons (Cell, 1997, 89, 755-764). Hematoxylin-eosin and Kossa staining (HE-Kossa staining) was carried out to dye the calcified site black to analyze the skeletons and calcification (Cell, 1997, 89, 755-764). The cartilage matrix was analyzed by safranin 0 staining and PAS staining (Cell, 1997, 89, 755-764). Apoptosis was analyzed by Tunnel staining (Cell, 1997, 89, 755-764). Cell proliferation was analyzed by Brdu staining (Cell, 1997, 89, 755-764). In situ hybridization was carried out in accordance with a conventional technique using know probes (Dev Dyn. April 1999, 214 (4), 279-90).

(4) Results and Discussion

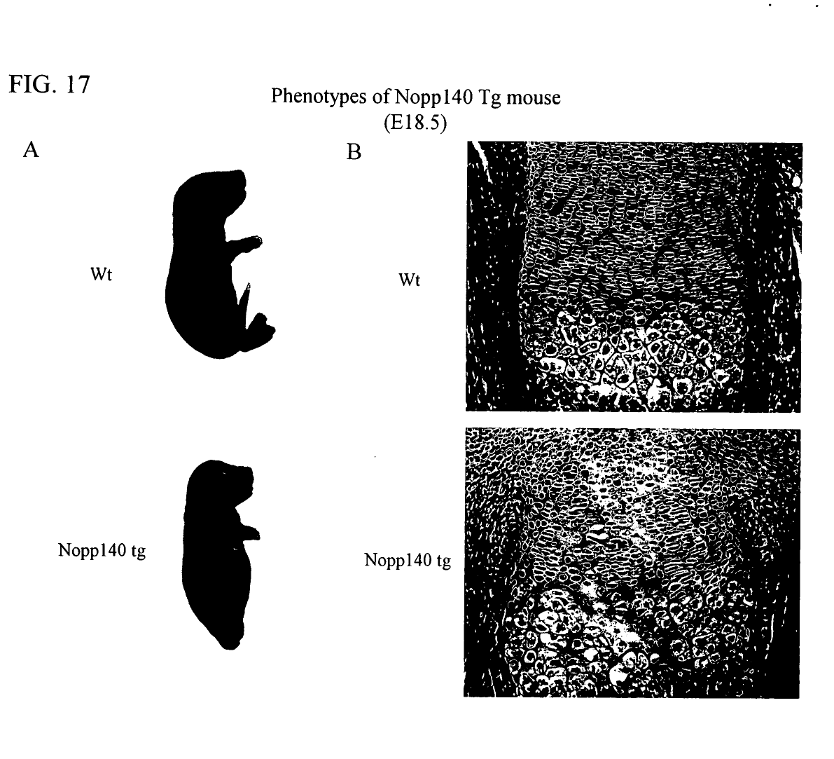

FIG. 17A shows the appearance of the Nopp140 transgenic mouse at day 18.5 of embryonic development, and FIG. 17B shows images of the HE-stained sections of joints between the tibias and the thighbone. Compared with a wild-type mouse (Wt), the Nopp140 transgenic mouse (Nopp140 tg) had significantly shorter limbs and smaller jaws (FIG. 17A, bottom row). As a result of HE staining, a disturbance was observed in a cell layer of the intrinsic proliferated chondrocytes of the Nopp140 transgenic mouse (Nopp140 tg), which suggests that differentiation was stimulated at this site (FIG. 17B, bottom row). This indicates that the Nopp140 gene may stimulate cartilage differentiation.

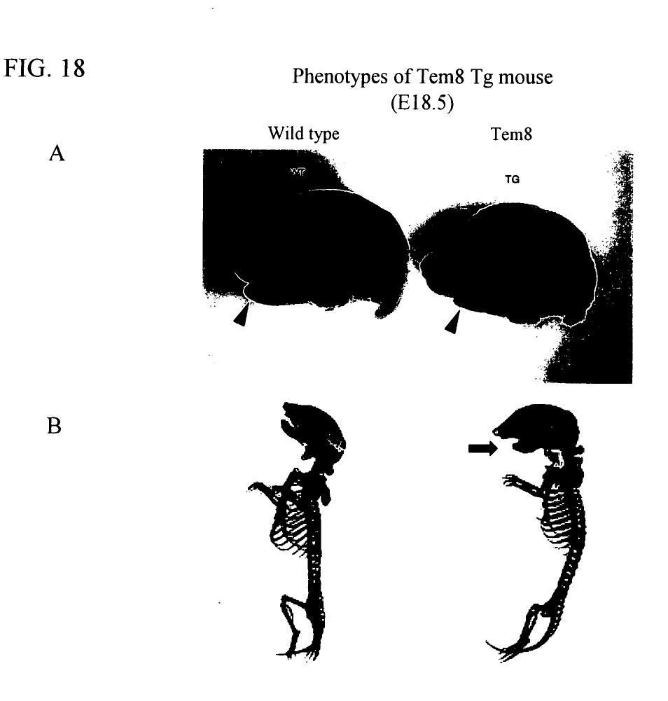

FIG. 18A shows the head appearance of the Tem8 transgenic mouse at day 18.5 of embryonic development, and FIG. 18B shows images of stained skeletons. Compared with a normal mouse (i.e., a wild-type mouse), the jaw of the Tem8 transgenic mouse (Tem8) was not sufficiently developed (FIG. 18A, right) and it had shorter limbs (FIG. 18B, right). By observation of HE-stained sections, calcification is observed at a site different from a site at which original bone formation takes place (i.e., allopatry). This indicates that abnormal cartilage differentiation takes place in such mouse. Compared with a wild-type mouse, tunnel-positive apoptosis took place in many more chondrocytes of the Tem8 transgenic mice. Such apoptosis-positive cells expressed osteopontin, and apoptosis involving abnormal cartilage differentiation was induced. This indicates that the Tem8 gene stimulates cartilage differentiation.

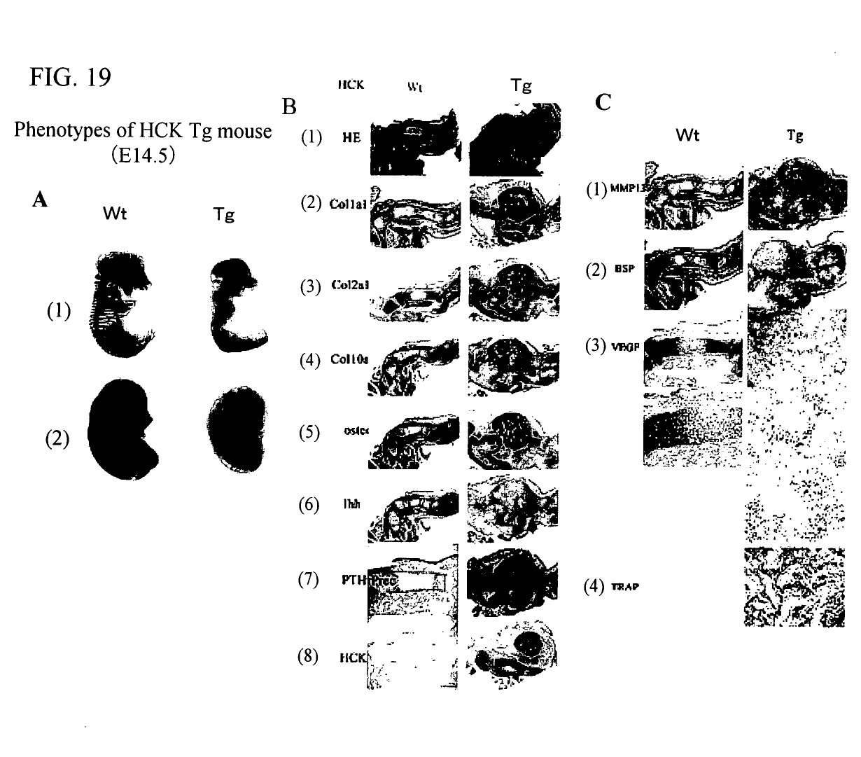

FIG. 19A-(2) shows the appearance of the HCK transgenic mouse at day 14.5 of embryonic development and FIG. 19A-(1) shows an image of the stained skeleton thereof. FIG. 19 A and B show images of HE staining (FIG. 19B-(1)), the results of expression analysis via in situ hybridization using the following probes: type I collagen (Collal: FIG. 19B-(2)), type II collagen (Col2al: FIG. 19B-(3)), type X collagen (CollOal: FIG. 19B-(4)), osteopontin (FIG. 19B-(5)), Indian hedgehog (FIG. 19B-(6): Ihh), a PTH receptor (FIG. 19B-(7): Pthrl), Hck (FIG. 19B-(8)), MMP13 (FIG. 19C-(1)), BSP (FIG. 19C-(2)), and VEGF (FIG. 19C-(3)), and images showing the results of analyzing osteoclasts by TRAP staining (FIG. 19C-(4)). The HCK transgenic mouse (Tg) had a smaller body size, a more protruding abdomen, and stubbier limbs compared with a wild-type mouse (Wt) (FIG. 19A-(2)). In the tibia of the HCK transgenic mouse, unorganized abnormal growth of cells inhibited the formation of the normal epiphyseal plate, and longitudinal growth was abnormal (FIG. 19A-(1)). Also, the number of chondrocytes with stimulated differentiation that express Ihh and Col10al was small (FIG. 19-(6) and (4)). Expression of Col2al and Pthr1 was observed in expanded tissue (FIG. 19-(3) and (7)). The expression level of the osteopontin that is a marker for the ultimately differentiated hypertrophic cartilage and osteoblasts was increased in cartilage tissues (FIG. 19B-(5)). Collal that is not generally expressed in cartilage was expressed in cartilage tissues (FIG. 19B-(2)). The expression level of BSP, which is usually expressed in osteoblasts, was decreased (FIG. 19C-(2)). The expression level of MMP 13 and that of VEGF, which are known to induce angioinvasion into cartilage, were increased (FIG. 19C-(1) and (3)). The TRAP-positive osteoclasts that are usually invasive to the ultimately differentiated chondrocytes were invasive to the undifferentiated chondrocytes (FIG. 19C-(4)). Brdu staining revealed that cell proliferation was enhanced in tissues.

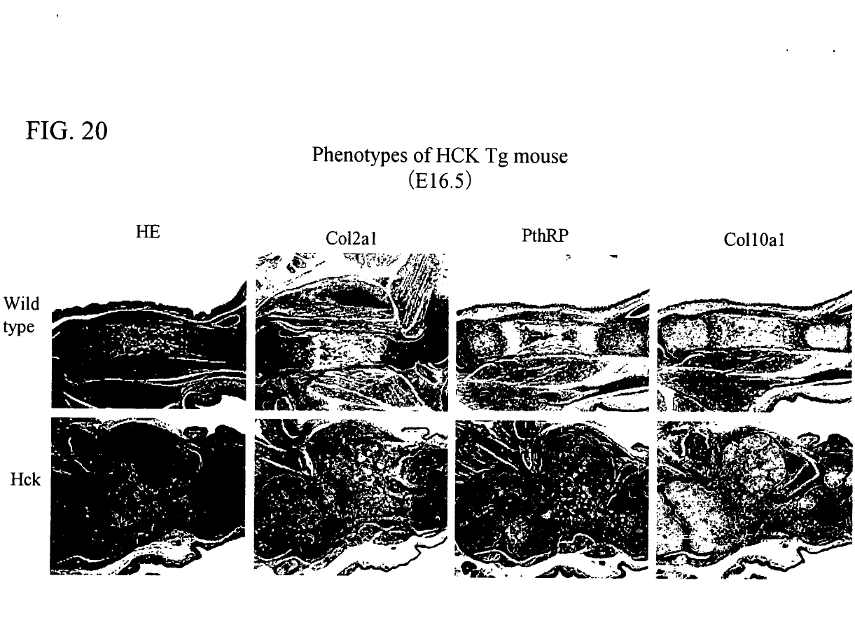

FIG. 20 shows the results of expression analysis by HE staining (HE) of the HCK transgenic mouse at day 16.5 of embryonic development and via in situ hybridization of type II collagen (Col2al), a PTH receptor (PthRP), and type X collagen (Col10al). In the tibia of the HCK transgenic mouse, unorganized abnormal growth of cells inhibited the formation of the normal epiphyseal plate, and longitudinal growth was abnormal (FIG. 20-HE, bottom row). Expression of Col2al was observed in expanded tissues (FIG. 20-Co10al, bottom row). The expression level of PthRP and that of Col10al that are generally observed in differentiated chondrocytes was lowered (FIG. 20-PthhRP and Col10al, bottom rows).

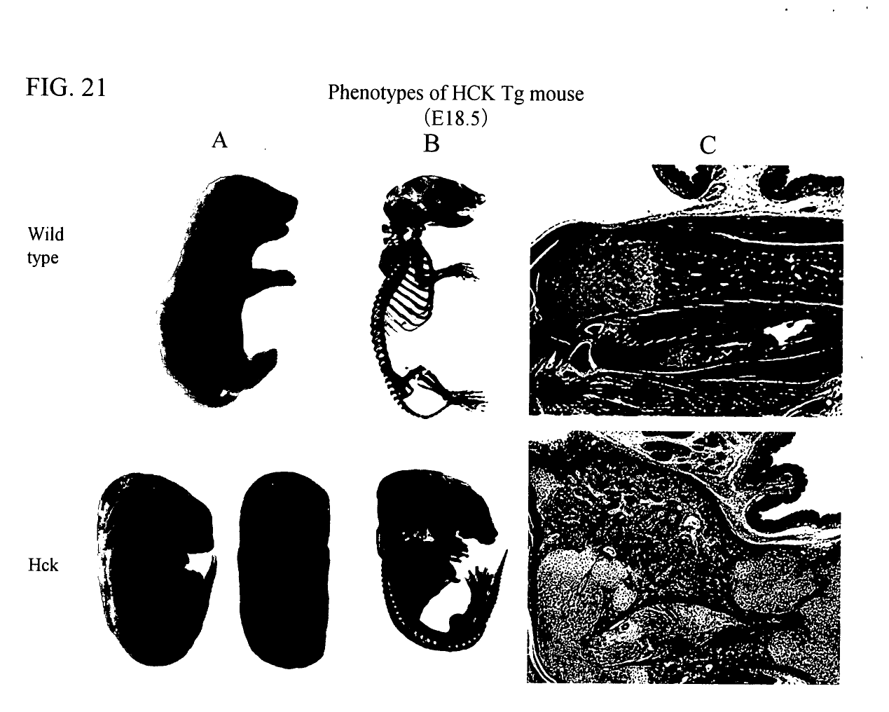

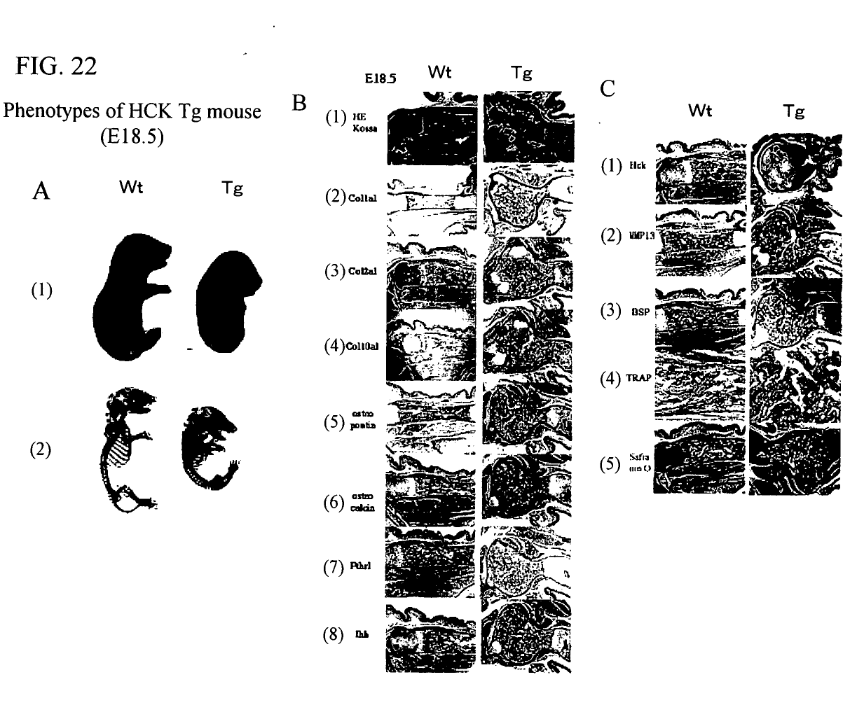

FIG. 21A shows the appearance of the HCK transgenic mouse at day 18.5 of embryonic development, FIG. 21B shows images of the stained skeletons thereof, and FIG. 21C shows the results of HE staining thereof FIG. 22A-(1) shows the appearance of the HCK transgenic mouse at day 18.5 of embryonic development and FIG. 22A-(2) shows an image of the stained skeleton thereof FIG. 22B and C show the results of expression analysis by HE and Kossa staining (FIG. 22B-(1); HE Kossa), images showing the results of expression analysis via in situ hybridization of type I collagen (FIG. 22B-(2): Collal), type II collagen (FIG. 22B-(3): Col2al), type X collagen (FIG. 22B-(4): Col10al), osteopontin (FIG. 22B-(5): osteopontin), osteocalcin (FIG. 22B-(6): osteocalcin), a PTH receptor (FIG. 22B-(7): Pthr1), Indian hedgehog (FIG. 22B-(8): Ihh), Hck (FIG. 22C-(1)), MMP13 (FIG. 22C-(2)), and BSP (FIG. 22C-(3)), the results of analyzing osteoclasts by TRAP staining (FIG. 22C-(4)), and the results of analyzing proteoglycan by safranin 0 staining (FIG. 22C-(5)). Compared with a wild-type mouse (Wt), the HCK transgenic (Tg) mouse had a smaller body size, a more protruding abdomen, and stubbier limbs (FIG. 22A-(1)). There was a cleavage between the nose and the upper jaw, and the process of fusion between the nose and the upper jaw was inhibited. The chondrocranium and the nasal cartilage were covered with enchondral regions of expanded membrane bones. The directions along the proximal-distal axis were lost in enchondral bones and abnormal configurations were observed. The growth plates were not organized, and the joints were fused. Skeleton staining revealed that the amount of calcified tissues stained with alizarin red decreased and that the amount of the extracellular matrix stained with alcian blue increased (FIG. 22A-(2)). As a result of HE staining, the chondrocytes of the HCK transgenic mouse were found to be immature, and the invasion and the proliferation of mesenchymal cells were observed in the vicinity thereof (FIG. 22B-(1)). The number of Col2al-expressing chondrocytes decreased, and the bones were formed while involving the invasion and the proliferation of mesenchymal cells without a normal procedure (FIG. 22B-(2)). The expression levels of Ihh, Pthr1, and Col10al were also lowered (FIG. 22B-(8), (7), and (4)). Collal was expressed in bone tissues, and osteopontin was wide spread distributed (FIG. 22B-(2) and (5)). In contrast, a marker for mature osteoblast, i.e., osteocalcin, was hardly expressed, which indicates that cells constituting the bone tissue were immature (FIG. 22B-(6)). A bone collar was not organized, and continuity was lost due to the invasion of numerous blood vessels and mesenchymal cells. Such invasion of numerous blood vessels and mesenchymal cells was considered to result from an increase in TRAP-positive osteoclasts (FIG. 22C-(4)). As a result of safranin 0 staining, proteoglycan in cartilage tissues was assumed to be normal (FIG. 22C-(5)). Accordingly, the HCK genes were considered to enhance the proliferation of chondrocytes and to inhibit the differentiation thereof.

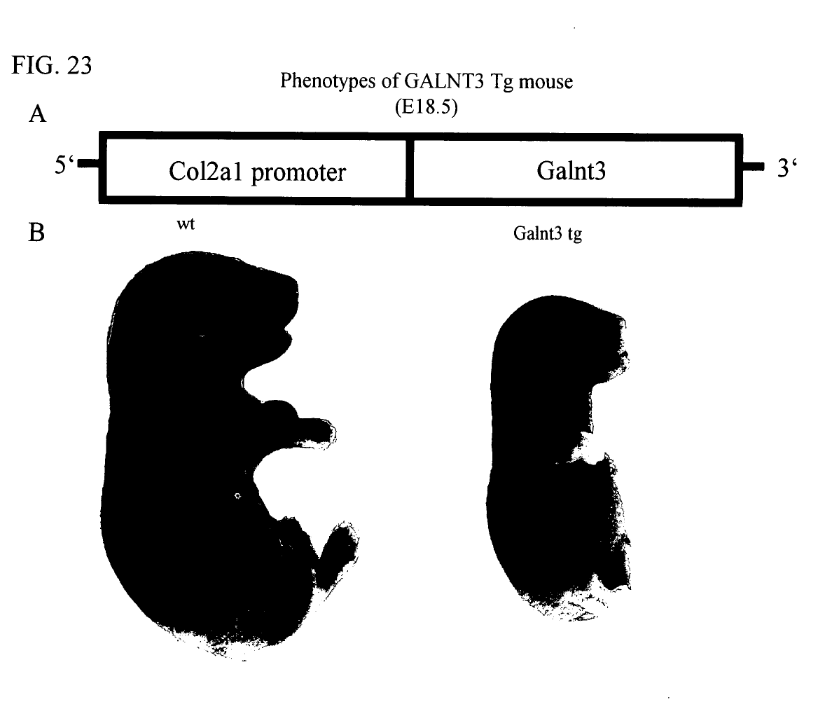

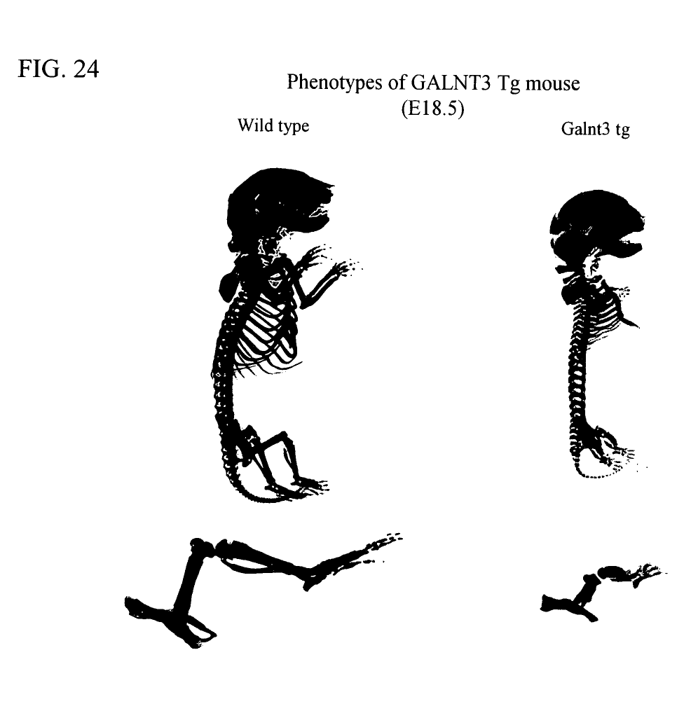

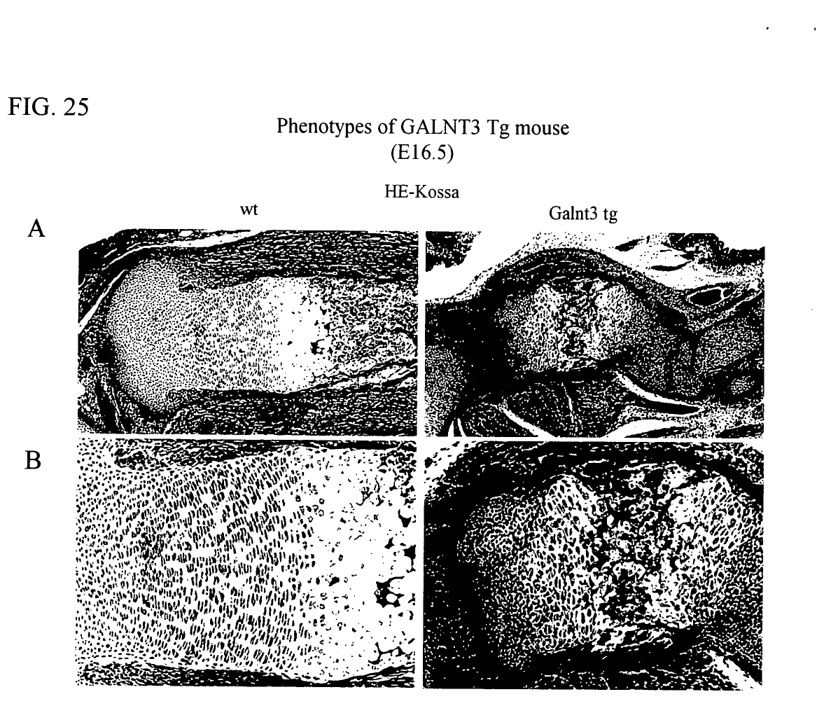

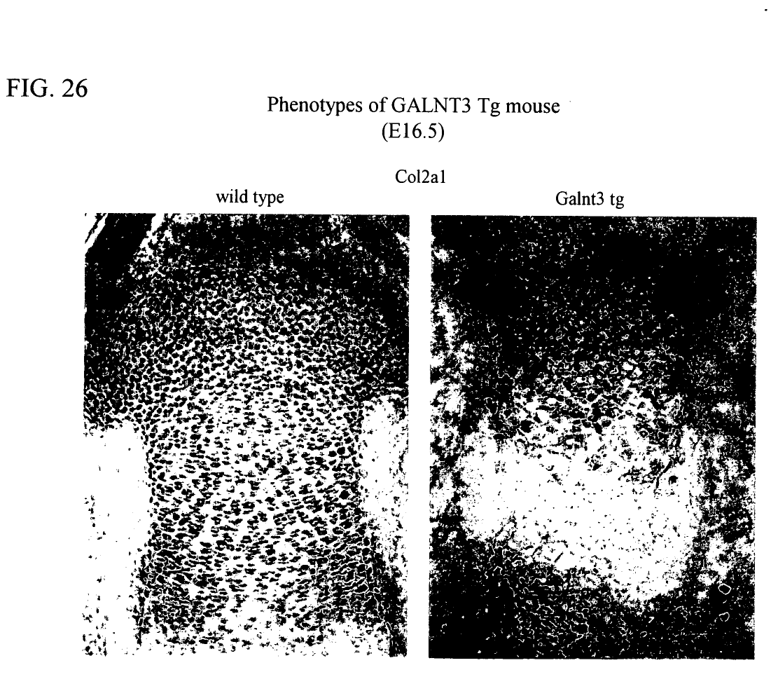

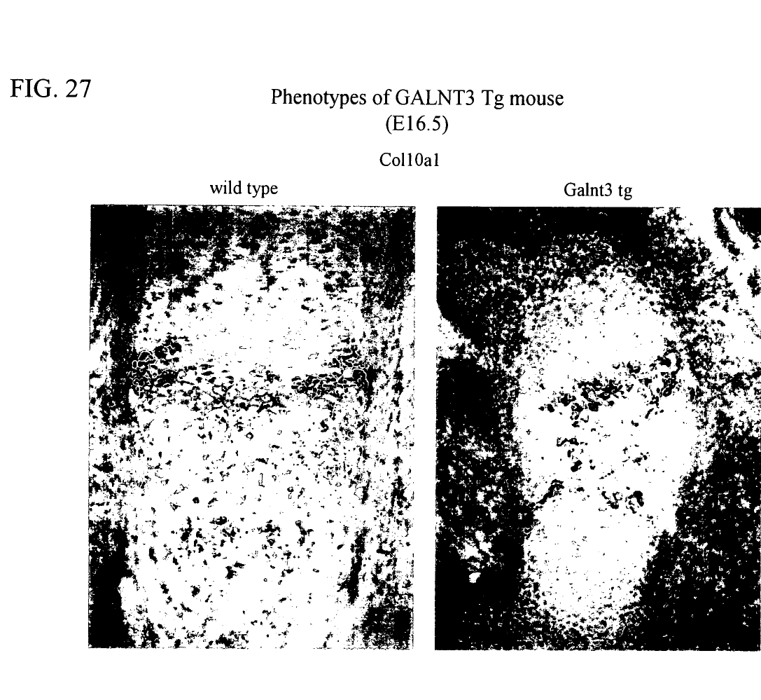

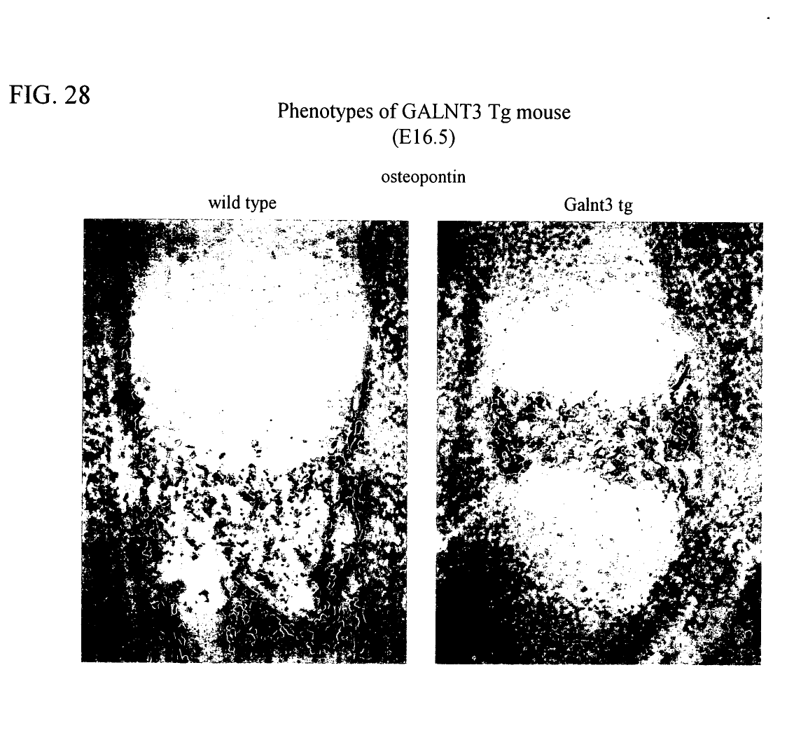

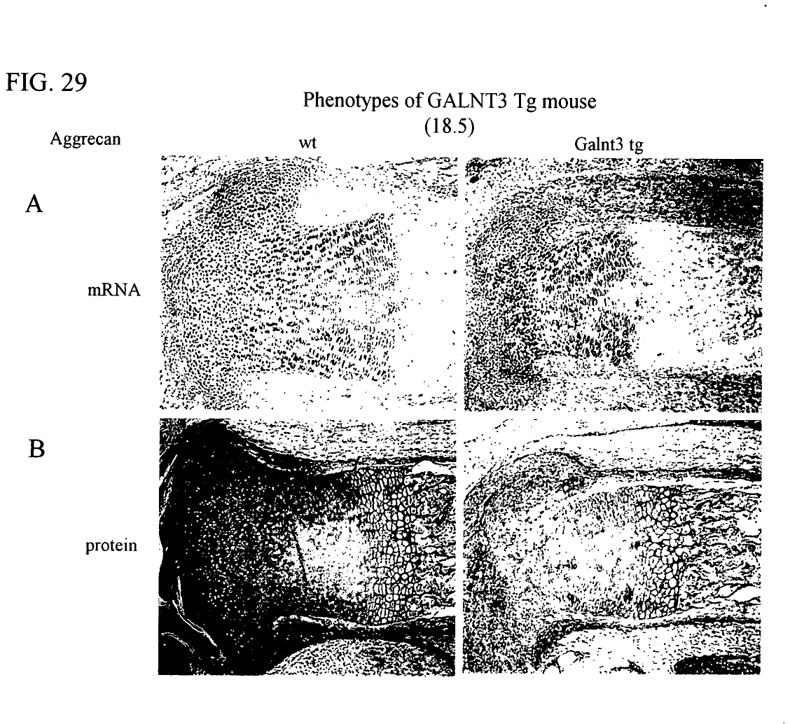

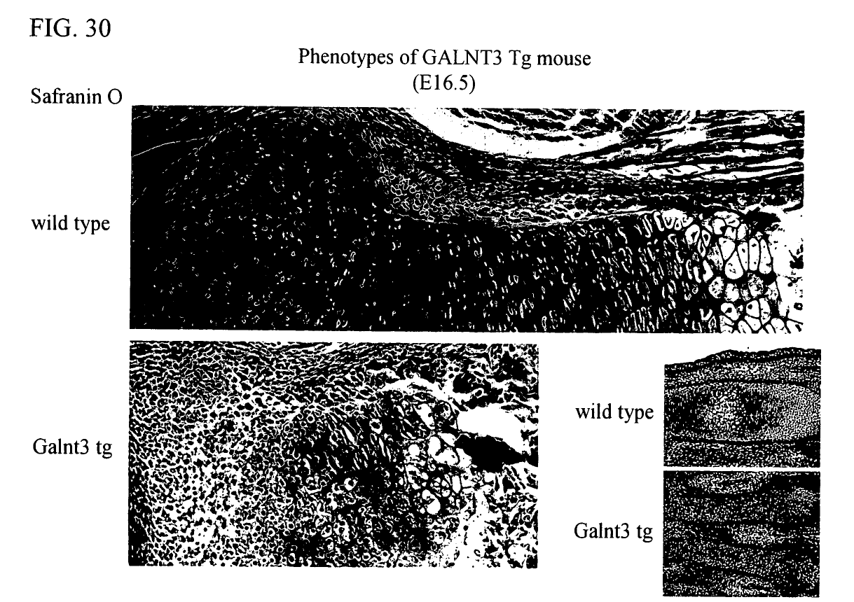

FIG. 23B shows the appearance of the GALNT3 transgenic mouse at day 18.5 of embryonic development. Compared with a wild-type mouse (Wt), the GALNT3 transgenic mouse (Galnt3 tg) had a smaller body size, shorter limbs, a smaller pectoral region, and a more protruding abdomen. FIG. 24 shows the result of skeleton staining. Compared with a normal (wild-type) mouse, the GALNT3 transgenic mouse (Galnt3 tg) had a smaller pectoral region and the significantly decreased bones calcified upon enchondral ossification. FIG. 25A and B show the results of HE and Kossa staining of the tibia ofthe GALNT3 transgenic mouse at day 16.5 of embryonic development (FIG. 25B is an enlarged image of FIG. 25A). Angioinvasion was observed in a wild-type mouse (Wt); however, it was delayed in the GALNT3 transgenic mouse (Galnt3 tg), and articular cavity formation was incomplete (FIG. 25, right). FIG. 26 shows the results of expression analysis via in silit hybridization of type II collagen (Col2al). Compared with a normal (wild-type) mouse, the GALNT3 transgenic mouse (Galnt3 tg) exhibited abnormal distribution of type II collagen expression. FIG. 27 shows the results of analysis via in situ hybridization of type X collagen, and FIG. 28 shows the results of analysis via in situ hybridization of osteopontin. Expression of type X collagen and that of osteopontin were observed in hypertrophic cartilage of both the wild-type mouse and the GALNT3 transgenic mouse (Galnt3 tg). FIG. 29A shows the results of analysis via in situ hybridization of mRNA expression of aggrecan (mRNA), and FIG. 29B shows an image of immunohistostaining representing expression at a protein level (protein). The mRNA expression of aggrecan was slightly increased in the GALNT3 transgenic mouse (Galnt3 tg) compared with the wild-type mouse (wt) (FIG. 29A), although aggrecan expression was decreased at the protein level (FIG. 29B). FIG. 30 shows the results of safranin 0 staining of the tibia at day 16.5 of embryonic development. Compared with a wild-type mouse, the staining affinity of the GALNT3 transgenic mouse (Galnt3 tg) was significantly deteriorated, which indicates that proteoglycan content was significantly decreased.

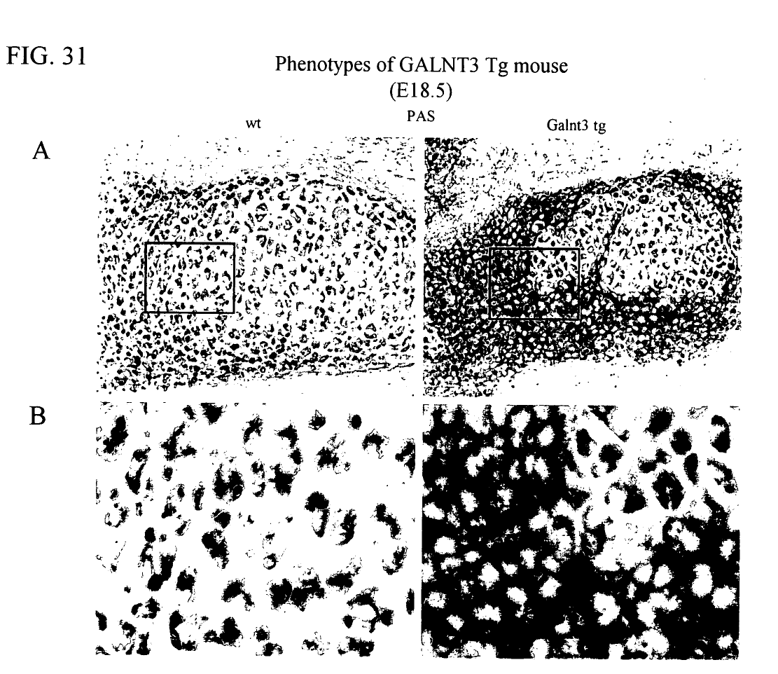

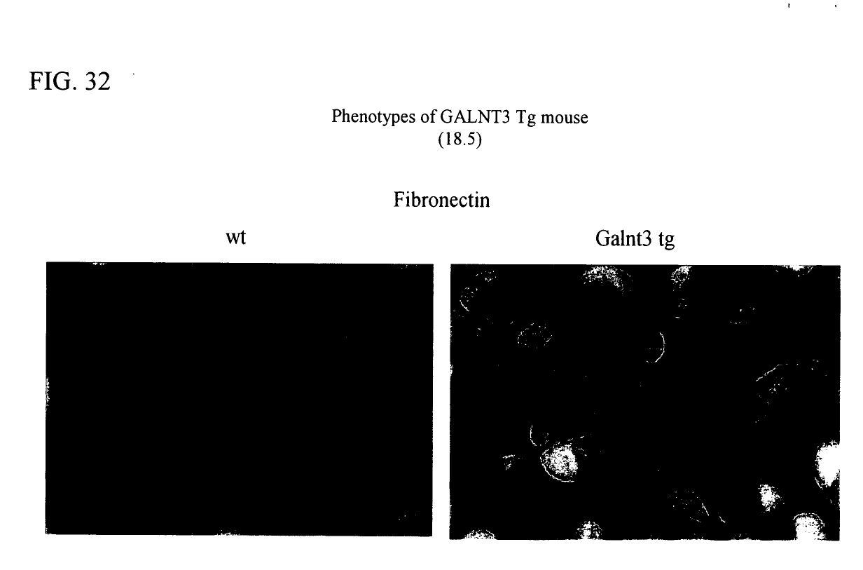

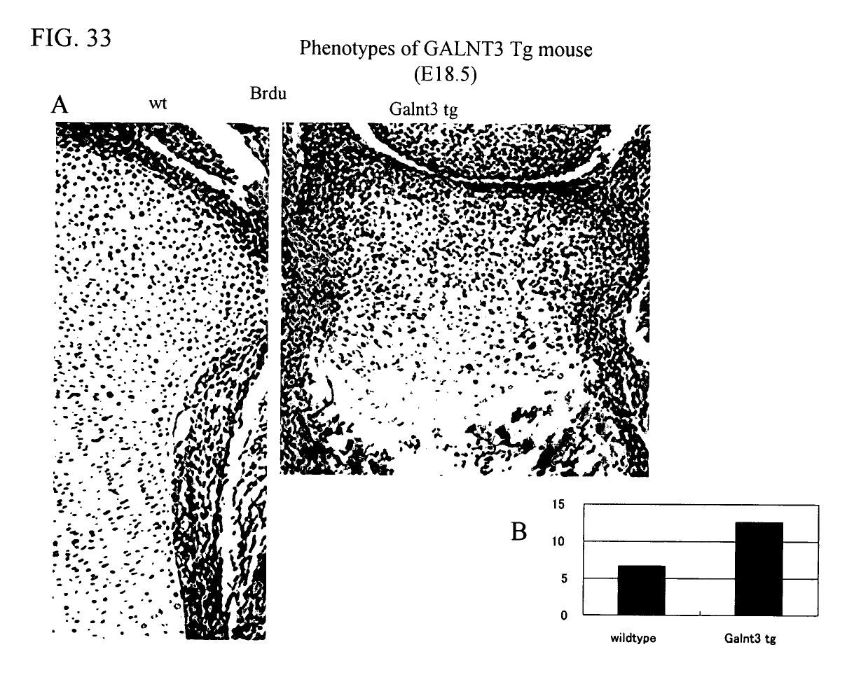

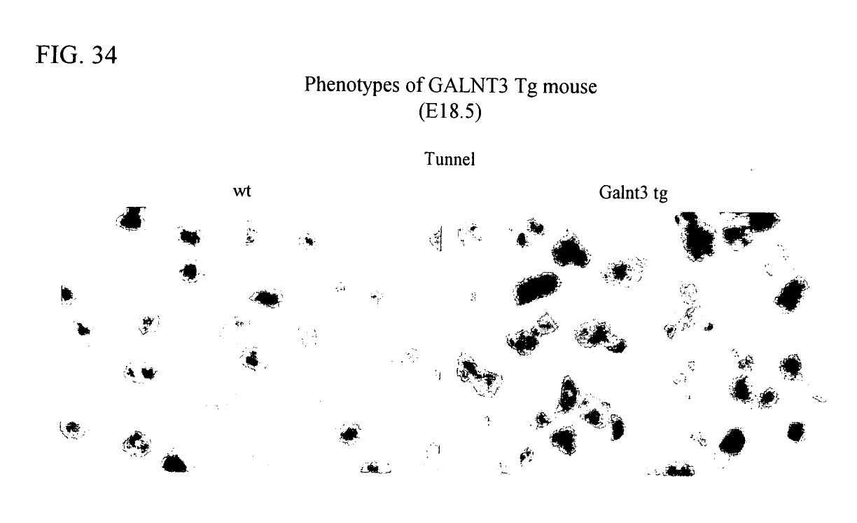

FIG. 31A and B show the results of PAS staining of the anklebone of the GALNT3 transgenic mouse at day 18.5 of embryonic development (FIG. 31B is an enlarged image of FIG. 31A). Compared with a wild-type mouse (wt), the staining affinity of the GALNT3 transgenic mouse (Galnt3 tg) was enhanced, which indicates that the level of mucin-like glycoproteins was increased. FIG. 32 shows an image of immunostained fibronectin in the tibia of the GALNT3 transgenic mouse at day 18.5 of embryonic development. Compared with a wild-type mouse, the distance between adjacent chondrocytes was small in the GALNT3 transgenic mouse, which indicates that the amount of extracellular matrix was decreased. FIG. 33A shows the results of analyzing by Brdu labeling the chondrocyte proliferation, and FIG. 33B is a graph showing the amount of BrdU incorporated. Compared with a wild-type mouse (wt), chondrocyte proliferation was enhanced in the GALNT3 transgenic mouse (Galnt3 tg). FIG. 34 shows the results of analysis by Tunnel staining. Compared with a wild-type mouse (wt), chondrocyte apoptosis was found to be enhanced in the TALNT3 transgenic mouse (Galnt3 tg). Accordingly, the GALNT3 genes were considered to be capable of inhibiting the formation of cartilage tissues. The joint cartilage is composed of a small amount of chondrocytes, i.e., 80% of the joint cartilage is accounted for by moisture and 20% thereof is accounted for by matrix. The matrix is composed of collagen (60% of the dry weight) and proteoglycan (10% of the dry weight). Proteoglyan consists of 95% polysaccharides referred to as “glucosaminoglycan” (GAG) and 5% proteins. When proteoglycan comprises a large quantity of water, it becomes a gelatinous material. Because of the water retentivity of proteoglycan, the water content of cartilage is as high as 80%, and this can impart elasticity and stiffness to the joint cartilage. When the water retentivity is lowered due to decreased proteoglycan content, cartilage elasticity becomes deteriorated, which may lead to tissue degeneration. Accordingly, inhibition of cartilage tissue generation and decreased proteoglycan content, which were observed in the GALNT3 transgenic mouse, were considered to result from changes associated with osteoarthritis.

Thus, the Nopp140 gene and the Tem8 gene stimulate cartilage differentiation to thereby allow the osteoarthritis development. On the contrary, the HCK gene inhibits cartilage differentiation to thereby inhibit osteoarthritis. The GALNT3 gene inhibits the formation of cartilage tissues to secondarily inhibit cartilage differentiation, although such gene may be involved in the advancement of osteoarthritis.

All publications, patents, and patent applications cited herein are incorporated herein by reference in their entirety.

### INDUSTRIAL APPLICABILITY

The present invention provides a method for obtaining a disease-associated gene by allowing a disease-associated transcription factor to express in a cell line that is deficient in such transcription factor or a primary cultured cell and a method for obtaining a bone and/or joint disease-associated gene, and preferably an osteoarthritis-associated gene, by allowing Runx2/Cbfa1 to express in the Runs2/Cbfa1-deficient chondrocyte cell line or primarily chondrocytes in culture. The downstream gene of Runx2/Cbfa1 obtained via such method and a polypeptide encoded thereby are capable of regulating cartilage differentiation, and they can also serve as pharmaceutical preparations for a bone and/or joint disease (preferably osteoarthritis). Further, the present invention provides a method for screening for a candidate compound of a pharmaceutical preparation for a bone and/or joint disease (preferably osteoarthritis) and an animal model of a bone and/or joint disease (preferably osteoarthritis). The methods and products provided by the present invention are significantly useful in the fields of clinical and basic pharmaceutical and medical products for a bone and/or joint disease (preferably osteoarthritis).

Sequence Listing Free Text

SEQ ID NOs: 53 to 114: synthetic oligonucleotides

1. A method for obtaining a disease-associated gene, wherein a disease-associated transcription factor is expressed in a cell line that is deficient in said transcription factor or in a primary cultured cell, and the gene the expression of which is thereby induced or inhibited is screened.

2. The method according to claim 1 wherein the disease-associated gene is a Runx2/Cbfa1-related disease-associated gene, and wherein Runx2/Cbfa1 is expressed in a Runxs/Cbfa1 -deficient chondrocyte cell line or in a Runx2/Cbfa1 -deficient primary cultured cell, and the gene the expression of which is thereby induced or inhibited is screened.

3. The method according to claim 2, wherein the Runx2/Cbfa1-related disease-associated gene is a gene associated with regulation of cartilage differentiation, and wherein Runx2/Cbfa1 is expressed in a Runx2/Cbfa1 -deficient chondrocyte cell line or in a Runx2/Cbfa1 -deficient primary cultured cell, and the gene the expression of which is thereby induced or inhibited is screened.

4. The method according to any one of claims 1 to 3, wherein said screening is carried out via subtraction or DNA chip analysis.

5. A primary chondrocyte or cultured chondrocyte derived from a Runx2/Cbfa1 -deficient mouse.

6. A chondrocyte derived from a Runx2/Cbfa1 - and p53-deficient mouse.

7. The chondrocyte cell line derived from the Runx2/Cbfa1 - and p53-deficient mouse according to claim 6, which is the RU-1 cell line or the RU-22 cell line deposited under the accession number FERM BP-10137 or FERM BP-10138 at the International Patent Organism Depositary of the National Institute of Advanced Industrial Science and Technology.

8. (canceled)

9. A polynucleotide having the nucleotide sequence shown in SEQ ID NO: 9.

10-14. (canceled)

15. A human homolog polynucleotide of the polynucleotide according to claim 9, which has the nucleotide sequence shown in SEQ ID NO: 35.

16. A polynucleotide having 65% or more homology to the polypeptide encoded by the polynucleotide having the nucleotide sequence shown in SEQ ID NO: 9 or 35, and encoding a protein capable of stimulating or inhibiting cartilage differentiation.

17. A polynucleotide being capable of hybridizing under stringent conditions to the polynucleotide having the nucleotide sequence shown in SEQ ID NO: 9 or, 35, or a complementary strand thereof, and encoding a protein capable of stimulating or inhibiting cartilage differentiation.

18. A recombinant DNA vector comprising the polynucleotide according to any one of claims 9, 15, 16, and 17 or a complementary strand thereof.

19. A transformant transformed with the recombinant DNA vector according to claim 18.

20. A polypeptide comprising the amino acid sequence shown in SEQ ID NO: 10.

21. A polypeptide comprising an amino acid sequence derived from the amino acid sequence shown in SEQ ID NO: 10 by deletion, substitution, or addition of one or several amino acid residues, and capable of stimulating or inhibiting cartilage differentiation.

22. A polypeptide comprising an amino acid sequence having at least 65% homology to the amino acid sequence shown in SEQ ID NO: 10, and capable of stimulating or inhibiting cartilage differentiation.

23-30. (canceled)

31. A pharmaceutical composition comprising the polynucleotide having the nucleotide sequence shown in SEQ ID NO: 1, 3, 5, 9, 15, 25, 27, 29, 31, 35, 41, or 51, a polynucleotide having 65% or more homology to the polypeptide encoded by the nucleotide sequence shown in SEQ ID NO: 1, 3, 5, 9, 15, 25, 27, 29, 31, 35, 41, or 51 and encoding a protein capable of stimulating or inhibiting cartilage differentiation, or a polynucleotide being capable of hybridizing under stringent conditions to the nucleotide sequence shown in SEQ ID NO: 1, 3, 5, 9, 15, 25, 27, 29, 31, 35, 41, or 51 and encoding a protein capable of stimulating or inhibiting cartilage differentiation, and a pharmaceutically acceptable carrier.

32. A method for preventing and/or treating a bone and/or joint disease comprising administering to a subject the polynucleotide having the nucleotide sequence shown in SEQ ID NO: 1, 3, 5, 9, 15, 25, 27, 29, 31, 35, 41, or 51, a polynucleotide having 65% or more homology to the polypeptide encoded by the nucleotide sequence shown in SEQ ID NO: 1, 3, 5, 9, 15, 25, 27, 29, 31, 35, 41, or 51 and encoding a protein capable of stimulating or inhibiting cartilage differentiation, or a polvnucleotide being capable of hybridizing under stringent conditions to the nucleotide sequence shown in SEQ ID NO: 1, 3, 5, 9, 15, 25, 27, 29, 31, 35, 41, or 51 and encoding a protein capable of stimulating or inhibiting cartilage differentiation.

33. The method according to claim 32, wherein the bone and/or joint disease is osteoarthritis.

34. A method for diagnosing a disease comprising contacting a sample with the polynucleotide having the nucleotide sequence shown in SEQ ID NO: 1, 3, 5, 9, 15, 25, 27, 29, 31, 35, 41, or 51, a nolmnucleotide having 65% or more homology to the polypeptide encoded by the nucleotide sequence shown in SEQ ID NO: 1, 3, 5, 9, 15, 25, 27, 29, 31, 35, 41, or 51 and encoding a protein capable of stimulating or inhibiting cartilage differentiation, or a polynucleotide being capable of hybridizing under stringent conditions to the nucleotide sequence shown in SEO ID NO: 1, 3, 5, 9, 15, 25, 27, 29, 31, 35, 41, or 51 and encoding a protein capable of stimulating or inhibiting cartilage differentiation.

35. A method for diagnosing a bone and/or joint disease comprising contacting a sample with the polynucleotide having the nucleotide sequence shown in SEQ ID NO: 1, 3, 5, 9, 15, 25, 27, 29, 31, 35, 41, or 51, a polynucleotide having 65% or more homology to the polypeptide encoded by the nucleotide sequence shown in SEQ ID NO: 1, 3, 5, 9, 15, 25, 27, 29, 31, 35, 41, or 51 and encoding a protein capable of stimulating or inhibiting cartilage differentiation, or a polynucleotide being capable of hybridizing under stringent conditions to the nucleotide sequence shown in SEQ ID NO: 1, 3, 5. 9, 15, 25, 27, 29, 31, 35, 41, or 51 and encoding a protein capable of stimulating or inhibiting cartilage differentiation.

36. The rmethod according to claim 35, wherein the bone and/or joint disease is osteoarthritis.

37. A transgenic animal model of a bone and/or joint disease, in which an expression level of the gene encoded by the polynucleotide having the nucleotide sequence shown in SEQ ID NO: 1, 3, 5, 9, 15, 25, 27, 29, 31, 35, 41, or 51. a polynucleotide having 65% or more homology to the polypeptide encoded by the nucleotide sequence shown in SEQ ID NO: 1, 3, 5, 9, 15, 25, 27, 29, 31, 35, 41, or 51 and encoding a protein capable of stimulating or inhibiting cartilage differentiation, or a polynucleotide being capable of hybridizing under stringent conditions to the nucleotide sequence shown in SEQ ID NO: 1, 3, 5, 9, 15, 25, 27, 29, 31, 35, 41, or 51 and encoding a protein capable of stimulating or inhibiting cartilage differentiation is enhanced or lowered.

38. A transgenic mouse model of a bone and/or joint disease, in which the gene encoded by the polynucleotide having the nucleotide sequence shown in SEO ID NO: 1, 3, 5, 9, 15, 25, 27, 29, 31, 35, 41, or 51, a polynucleotide having 65% or more homology to the polypeptide encoded by the nucleotide sequence shown in SEQ ID NO: 1, 3, 5, 9, 15, 25, 27, 29, 31, 35, 41, or 51 and encoding a protein capable of stimulating or inhibiting cartilage differentiation, or a polynucleotide being capable of hybridizing under stringent conditions to the nucleotide sequence shown in SEQ ID NO: 1, 3, 5, 9, 15, 25, 27, 29, 31, 35, 41, or 51 and encoding a protein capable of stimulating or inhibiting cartilage differentiation is expressed with the use of a type II collagen promoter.

39-94. (canceled)
